(19) United States
(12) Patent Application Publication
(10) Pub. No.: US 2025/0171433 A1
(43) Pub. Date: May 29, 2025

(54) A1H-BENZO[C][1,2]THIADIAZINE-2,2-DIOXIDES BEARING HETEROCYCLIC LINKERS AS SELECTIVE HISTONE DEACETYLASE 6 INHIBITORS

(71) Applicant: Georgetown University, Washington, DC (US)

(72) Inventors: Alan P. Kozikowski, Chicago, IL (US); Werner Tueckmantel, Madison, WI (US)

(73) Assignee: Georgetown University, Washington, DC (US)

(21) Appl. No.: 18/836,611

(22) PCT Filed: Feb. 8, 2023
(86) PCT No.: PCT/US2023/062226

§ 371 Date: Aug. 7, 2024

Related U.S. Application Data
(60) Provisional application No. 63/307,932, filed on Feb. 8, 2022.

(51) Int. Cl. C07D 417/14; A61K 31/5415; A61K 45/06

(52) U.S. Cl. CPC C07D 417/14; A61K 31/5415; A61K 45/06

(57) ABSTRACT

Histone deacetylases inhibitors (HDACbls) and compositions containing the same are disclosed. Methods of treating diseases and conditions wherein inhibition of HDAC6 provides a benefit, like a cancer, a neurodegenerative disorder, a neurological disease including peripheral neuropathies such as Charcot Marie Tooth disease, traumatic brain injury, stroke, malaria, an autoimmune disease, autism, and inflammation, also are disclosed.

### RELATED APPLICATIONS

This application claims the benefit of priority to U.S. Provisional Patent Application No. 63/307,932, filed Feb. 8, 2022; the contents of which are hereby incorporated by reference in their entirety.

### STATEMENT OF GOVERNMENT SUPPORT

This invention was made with government support under grant numbers 5R01NS079183, R41 AG058283, and R43HD093464 awarded by the National Institutes of Health. The government has certain rights in this invention.

### FIELD OF THE INVENTION

The present invention relates to histone deacetylase inhibitors selective for histone deactylase 6 (HDAC6Is), to pharmaceutical compositions comprising one or more of the HDAC6Is, to methods of increasing the sensitivity of cancer cells to the cytotoxic effects of radiotherapy and/or chemotherapy comprising contacting the cell with one or more of the HDAC6Is, and to therapeutic methods of treating conditions and diseases wherein inhibition of HDAC6 provides a benefit, for example, a cancer, an inflammation, a neurological disease, a neurodegenerative disorder, stroke, traumatic brain injury, allograft rejection, autoimmune diseases, and malaria, comprising administering a therapeutically effective amount of a present HDAC6I to an individual in need thereof. Preferentially, compounds of this invention may be useful for the treatment of peripheral neuropathies such as Charcot Marie Tooth disease and various other neurodegenerative disorders including dementia of the Alzheimer's type.

### BACKGROUND

The histone deacetylase HDAC protein family consists of at present 18 enzymes which are classified into four subgroups according to their homology to the yeast family. HDAC1, 2, 3, and 8—categorized as class I HDACs according to their homology with yeast Rpd3—are characterized by ubiquitous expression and localization to the nucleus. Class II HDACs show tissue-specific expression and shuttle between the nucleus and cytoplasm. Homologous to yeast Hda1, these enzymes are subdivided in class IIa (HDAC4, 5, 7, and 9) and class IIb (HDAC6 and 10). HDAC11, the only member of the class IV subfamily, shows similarities to the catalytic domains of both class I and II enzymes. Class I, II, and IV HDACs require Zn²⁺ as a cofactor of the deacetylating activity and are also referred to as the conventional HDACs. The sirtuins 1-7 are dependent on nicotinamide adenine dinucleotide for their activity and form class III of the HDACs.

HDAC inhibitor drugs hold tremendous possibilities in treating diverse diseases. Within the HDAC field, there exist a plethora of HDAC inhibitor (HDACI) compounds that are able to block the deacetylase enzymes. The majority of HDACI compounds inhibit across more than one class of HDAC enzymes and are thus labeled pan-inhibitors. It has proved challenging to discover HDACI compounds that are highly selective for a given subtype such as HDAC6. To date only unselective pan-HDACI compounds, e.g., Vorinostate® and Panbinostat® have advanced to the marketplace. However, pan-HDACIs are associated with dose limiting side effects and therefore have been limited to use in cancer therapy. Of the various HDAC isoforms that appear to be promising therapeutic targets for treating human diseases beyond cancer, HDAC6 has emerged as a particularly attractive target (Simoes-Pires, C. et al., Mol. Neurodegener. 2013; 8: 7). For example, HDAC6Is have shown potential to treat peripheral neuropathies such as Charcot Marie Tooth (CMT) disease (d'Udewalle, C. et al., Nat. Med. 2011, 17(8), 968-74), the leading genetic neuropathy, and tauopathies including Alzheimer's disease (AD) (Selenica, M. L. et al., Alzheimers Res. Ther. 2014 6(1):12). Furthermore, HDAC6Is are expected to be safe for treatment of non-cancer diseases with chronic dosing since HDAC6 knockout animals remain viable. HDAC6 is involved primarily in regulating the acetylation status of cytosolic proteins such as α-tubulin, HSP-90, cortactin, HSF-1, and other protein targets. This enzyme also plays a role in the recognition and clearance of polyubiquitinated misfolded proteins from the cell through aggresome formation. The development of HDAC6 selective compounds has recently been reviewed (Kalin, J. H. et al., J. Med. Chem. 2013, 56, 6297-6313). In general, HDACIs are comprised of three main motifs: a zinc binding group (ZBG), a cap group, and a linker that bridges the previous two (FIG. 1). A properly optimized cap group can improve both potency and selectivity, presumably through its ability to engage in appropriate contacts with residues on the enzyme surface.

Many HDACIs such as trichostatin A (TSA) and SAHA contain a hydroxamic acid function as ZBG (FIG. 1). Many of the hydroxamic acid-based HDAC inhibitors including the marketed drug Vorinostat® have been found to be genotoxic. Genotoxicity is an undesired side effect that limits a drug's scope to diseases that can be considered to be life threatening such as cancers. Certainly, for use in non-cancer diseases that would require chronic, longer term dosing it would be essential to have HDACI compounds that are not genotoxic. As such, there is a great need for the discovery of potent and selective HDACIs, in particular HDAC6Is that do not show genotoxic activity.

Extensive evidence supports a therapeutic use for HDAC6Is in the treatment of a variety of conditions and diseases including neurodegeneration. However, despite exhibiting overall beneficial effects, like beneficial neuroprotective effects, for example, HDAC6Is known to date have little specificity with regard to HDAC inhibition, and therefore may inhibit two or more of the zinc-dependent histone deacetylases. It is still unknown which is (are) the salient HDAC(s) that are able to confer neuroprotection. Emerging evidence suggests that at least some of the HDAC isozymes are absolutely required for the maintenance and survival of neurons, e.g., HDAC1. Additionally, adverse side effect issues have been noted with nonspecific HDAC inhibition. Thus, the clinical efficacy of present-day nonspecific HDAC6Is for stroke, neurodegenerative disorders, neurological diseases, and other diseases and conditions ultimately may be limited. It is important therefore to design, synthesize, and test compounds capable of serving as potent, and isozyme-selective, HDAC6Is that can ameliorate the effects of neurological diseases, neurodegenerative disorders, peripheral neuropathies including chemotherapy induced neuropathies, traumatic brain injury, cancer, inflammation, malaria, autoimmune diseases, and other conditions and diseases mediated by HDAC6.

An important advance in the art would be the discovery of HDAC6Is, and particularly selective HDAC6Is, that are useful in the treatment of diseases wherein HDAC6 inhibition provides a benefit, such as cancers, neurological diseases, traumatic brain injury, neurodegenerative disorders and other peripheral neuropathies, stroke, hypertension, malaria, allograft rejection, rheumatoid arthritis, and various inflammatory conditions. Accordingly, a significant need exists in the art for efficacious compounds, compositions, and methods useful in the treatment of such diseases, alone or in combination with other therapies used to treat these diseases and conditions. The present invention is directed to meeting this need.

### SUMMARY OF THE INVENTION

The present invention relates to histone deacetylase 6 inhibitors (HDAC6Is), pharmaceutical compositions comprising the HDAC6I, and methods of treating diseases and conditions wherein inhibition of HDAC6 provides a benefit, such as a cancer, a neurological disease, a psychiatric illness, a neurodegenerative disorder, a peripheral neuropathy, stroke, hypertension, inflammation, traumatic brain injury, rheumatoid arthritis, allograft rejection, sepsis, and autoimmune diseases, comprising administering a therapeutically effective amount of an HDAC6I to an individual in need thereof. The present invention also relates to a method of increasing the sensitivity of a cancer cell to radiotherapy and/or chemotherapy. The present invention also allows for the use of these HDAC6Is in combination with other drugs and/or therapeutic approaches. In some embodiments, the present HDAC6Is exhibit selectivity in particular over certain other HDAC isozymes, e.g., HDAC1. In particular, the invention concerns the discovery of compounds containing the tetrahydroquinoline moiety or an analog thereof as the cap residue.

More particularly, the present invention relates to histone deacetylase 6 inhibitors (HDAC6Is) having General Formula I:

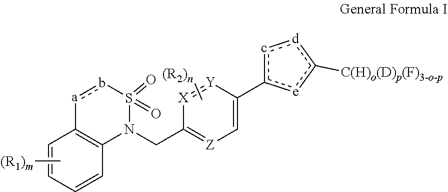

or a pharmaceutically acceptable salt and/or solvate thereof, wherein:
[symbol] represents a single or double bond; in the case of the five-membered ring moiety, there are two double bonds and three single bonds, together arranged in such manner as to give rise to a five-membered ring heteroaryl group;
X and Y are independently carbon or nitrogen, and Z is N or C(F);
m=0, 1, or 2; except when R₁ is deuterium, m is an integer ranging from 0 to 4;
n=0, 1, or 2 if at least one of X and Y is carbon; or n=0 or 1 if both X and Y are nitrogen; the sum of o and p is 0 or 1;
R₁ and R₂ are independently selected from the group consisting of hydrogen, deuterium, halogen, hydroxyl, —CH₂OH, —CH₂CH₂OH, cyano, —NRₐR_{b}, —CH₂NRₐR_{b}, —C(O)NRₐR_{b}, —S(O)₂NRₐR_{b}, acetyl, C₁-C₆ alkyl, C₁-C₆ alkenyl, C₁-C₆ alkynyl, C₁-C₆ alkoxy, C₁-C₆ haloalkyl, F₃C—S—, C₃-C₆ cycloalkyl, (C₃-C₅ cycloalkyl)-(C₁-C₃ alkyl)-, (C₁-C₃ alkyl)-(C₃-C₅ cycloalkyl)-, (C₃-C₅ cycloalkyl)-O—, aryl, aryl-O—, aryl-(C₁-C₃ alkyl)-, aryl-(C₁-C₃ alkoxy)-, heteroaryl, heteroaryl-O—, heteroaryl-(C₁-C₃ alkyl)-, heteroaryl-(C₁-C₃ alkoxy)-, 4- to 6-membered heterocyclyl, or (4- to 6-membered heterocyclyl)-(C₁-C₃ alkyl)-; or two of R₁ and/or two of R₂ are attached to adjacent carbon atoms of their respective aromatic rings and are connected to form a five- or six-membered carbo- or heterocyclic ring;
Rₐ and R_{b} are independently selected from the group consisting of hydrogen and C₁-C₆ alkyl or C₃-C₆ cycloalkyl, or these groups may be joined to form a 3- to 7-membered heterocyclyl;
a is CR_{c}R_{d}, C═O, NRₑ, O, or S when [symbol] represents a single bond, or CR or N when [symbol] represents a double bond;
b is CR_{c}R_{d} when [symbol] represents a single bond or CR_{c} when [symbol] represents a double bond, or b is N when [symbol] represents a double bond and a is CR_{c};
R_{c} and R_{d} are independently hydrogen, deuterium, fluorine, chlorine, C₁-C₆ alkyl, or C₃-C₆ cycloalkyl, or they are joined together to form a 3- to 6-membered cycloalkyl;
Rₑ is selected from the group consisting of hydrogen, C₁-C₆ alkyl, C₃-C₆ cycloalkyl, aryl, heteroaryl, or 4- to 6-membered heterocyclyl;
one of c, d, and e is oxygen, and the remainder are nitrogen; or c is C(R_{f}), one of d and e is oxygen, and the other is nitrogen; and
R_{f} is hydrogen, deuterium, fluorine, chlorine, methyl, difluoromethyl, trifluoromethyl, or cyano.

### BRIEF DESCRIPTION OF THE DRAWINGS

FIG. 1 shows structures of SAHA and TSA, and general structure of an HDAC inhibitor.

FIGS. 2A, 2B, 3A, 3B and 4 show the biological activity of exemplary compounds disclosed herein.

FIG. 5 shows an exemplary method of synthesizing compound 51.

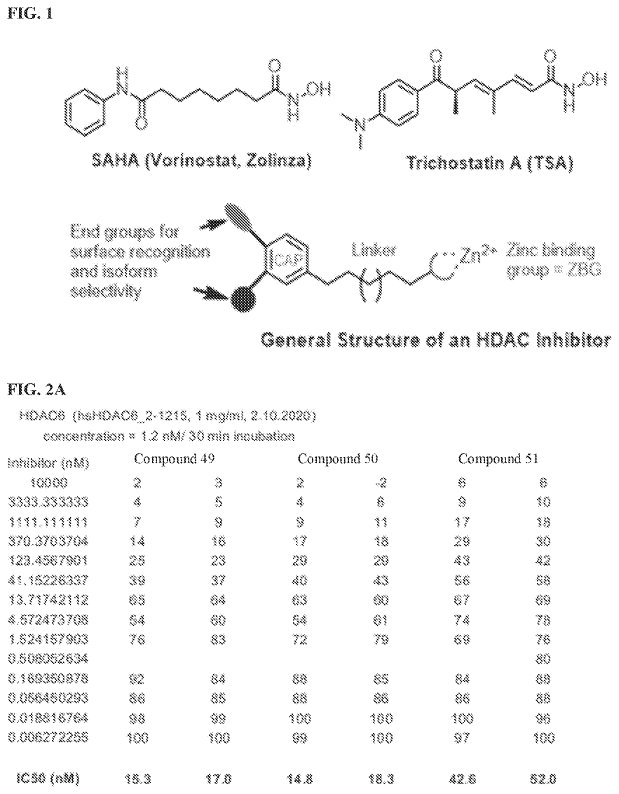

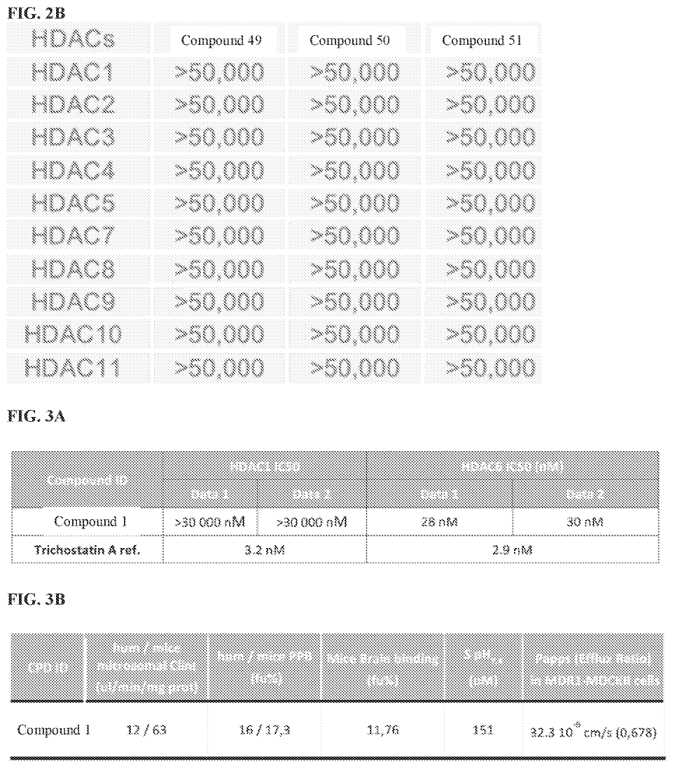

### DETAILED DESCRIPTION OF THE INVENTION

In particular, the present invention is directed to HDAC6Is, compositions comprising the present HDAC6I, and therapeutic uses of the HDAC6Is of General Formula I:

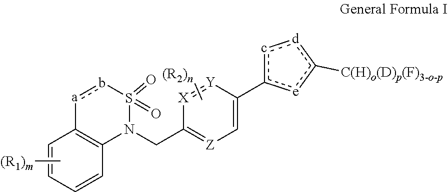

or a pharmaceutically acceptable salt and/or solvate thereof, wherein:
[symbol] represents a single or double bond; in the case of the five-membered ring moiety, there are two double bonds and three single bonds, together arranged in such manner as to give rise to a five-membered ring heteroaryl group;
X and Y are independently carbon or nitrogen, and Z is N or C(F);
m=0, 1, or 2; except when R₁ is deuterium, m is an integer ranging from 0 to 4;
n=0, 1, or 2 if at least one of X and Y is carbon; or n=0 or 1 if both X and Y are nitrogen; the sum of o and p is 0 or 1;
R₁ and R₂ are independently selected from the group consisting of hydrogen, deuterium, halogen, hydroxyl, —CH₂OH, —CH₂CH₂OH, cyano, —NRₐR_{b}, —CH₂NR_{c}R_{b}, —C(O)NRₐR_{b}, —S(O)₂NRₐR_{b}, acetyl, C₁-C₆ alkyl, C₁-C₆ alkenyl, C₁-C₆ alkynyl, C₁-C₆ alkoxy, C₁-C₆ haloalkyl, F₃C—S—, C₃-C₆ cycloalkyl, (C₃-C₅ cycloalkyl)-(C₁-C₃ alkyl)-, (C₁-C₃ alkyl)-(C₃-C₅ cycloalkyl)-, (C₃-C₅ cycloalkyl)-O—, aryl, aryl-O—, aryl-(C₁-C₃ alkyl)-, aryl-(C₁-C₃ alkoxy)-, heteroaryl, heteroaryl-O—, heteroaryl-(C₁-C₃ alkyl)-, heteroaryl-(C₁-C₃ alkoxy)-, 4- to 6-membered heterocyclyl, or (4- to 6-membered heterocyclyl)-(C₁-C₃ alkyl)-; or two of R₁ and/or two of R₂ are attached to adjacent carbon atoms of their respective aromatic rings and are connected to form a five- or six-membered carbo- or heterocyclic ring;
Rₐ and R_{b} are independently selected from the group consisting of hydrogen and C₁-C₆ alkyl or C₃-C₆ cycloalkyl, or these groups may be joined to form a 3- to 7-membered heterocyclyl;
a is CR_{c}R_{d}, C═O, NRₑ, O, or S when [symbol] represents a single bond, or CR or N when [symbol] represents a double bond;
b is CR_{c}R_{d} when [symbol] represents a single bond or CR_{c} when [symbol] represents a double bond, or b is N when [symbol] represents a double bond and a is CR_{c};
R_{c} and R_{d} are independently hydrogen, deuterium, fluorine, chlorine, C₁-C₆ alkyl, or C₃-C₆ cycloalkyl, or they are joined together to form a 3- to 6-membered cycloalkyl;
Rₑ is selected from the group consisting of hydrogen, C₁-C₆ alkyl, C₃-C₆ cycloalkyl, aryl, heteroaryl, or 4- to 6-membered heterocyclyl;
one of c, d, and e is oxygen, and the remainder are nitrogen; or c is C(R_{f}), one of d and e is oxygen, and the other is nitrogen; and
R_{f} is hydrogen, deuterium, fluorine, chlorine, methyl, difluoromethyl, trifluoromethyl, or cyano.

In an embodiment, this invention comprises HDAC6Is of formula Ib

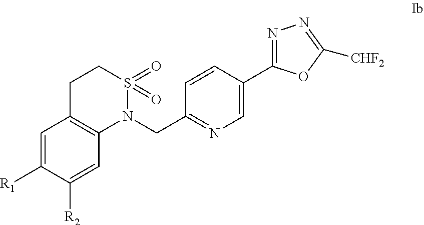

wherein R₁ and R₂ are defined as in Formula I.

In another embodiment, this invention comprises HDAC6Is of formula Ib wherein R₁ and R₂ are independently selected from H, D, Cl, and F.

Another embodiment comprises HDAC6Is of formula Ic

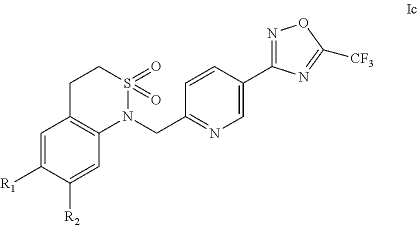

wherein R₁ and R₂ are defined as in Formula I.

In another embodiment, this invention comprises HDAC6Is of formula Ic wherein R₁ and R₂ are independently selected from H, D, Cl, and F.

In another embodiment, this invention comprises HDAC6Is of formula Id

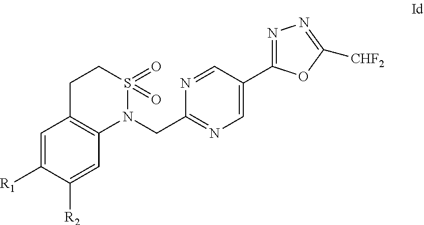

wherein R₁ and R₂ are defined as in formula I.

In another embodiment, this invention comprises HDAC6Is of formula Id, wherein R₁ and R₂ are independently selected from H, D, Cl, and F.

In another embodiment, this invention comprises HDAC6Is of formula Ie

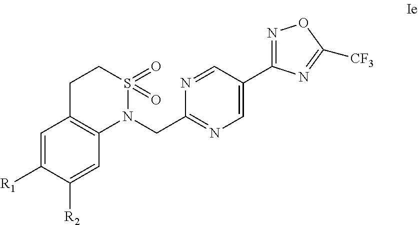

wherein R₁ and R₂ are defined as in formula I.

In another embodiment, this invention comprises HDAC6Is of formula Ie, wherein R₁ and R₂ are independently selected from H, D, Cl, and F.

In another embodiment, this invention comprises HDAC6Is of formula If

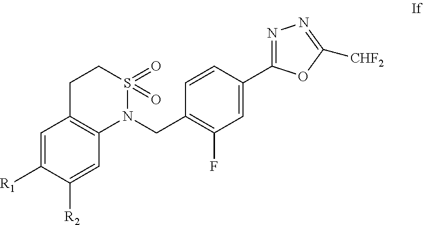

wherein R₁ and R₂ are defined as in formula I.

In another embodiment, this invention comprises HDAC6 Is of formula If, wherein R₁ and R₂ are independently selected from H, D, Cl, and F.

In another embodiment, this invention comprises HDAC6Is of formula Ig

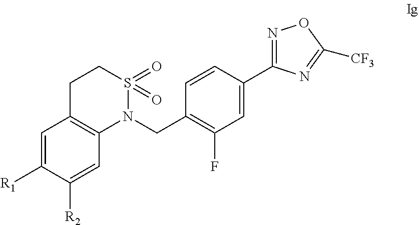

wherein R₁ and R₂ are defined as in formula I.

In another embodiment, this invention comprises HDAC6Is of formula Ig, wherein R₁ and R₂ are independently selected from H, D, Cl, and F.

In another embodiment, this invention comprises HDAC6Is of formula Ih

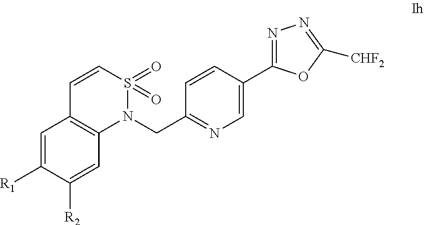

wherein R₁ and R₂ are defined as in formula I.

In another embodiment, this invention comprises HDAC6Is of formula Ih, wherein R₁ and R₂ are independently selected from H, D, Cl, and F.

In another embodiment, this invention comprises HDAC6Is of formula Ii

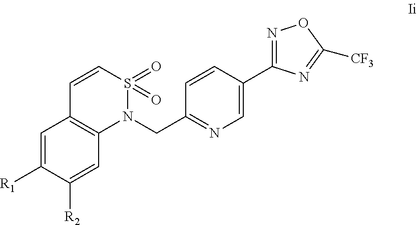

wherein R₁ and R₂ are defined as in formula I.

In another embodiment, this invention comprises HDAC6Is of formula Ii, wherein R₁ and R₂ are independently selected from H, D, Cl, and F.

In another embodiment, this invention comprises HDAC6Is of formula Ij

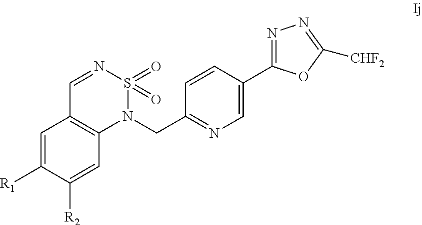

wherein R₁ and R₂ are defined as in formula I.

In another embodiment, this invention comprises HDAC6Is of formula Ij, wherein R₁ and R₂ are independently selected from H, D, Cl, and F.

In another embodiment, this invention comprises HDAC6Is of formula Ik

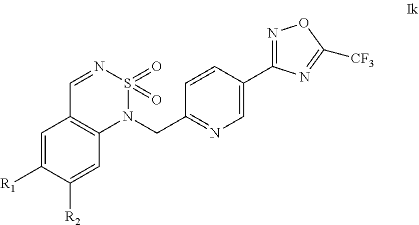

wherein R₁ and R₂ are defined as in formula I.

In another embodiment, this invention comprises HDAC6Is of formula Ik, wherein R₁ and R₂ are independently selected from H, D, Cl, and F.

In another embodiment, this invention comprises HDAC6Is of formula IL

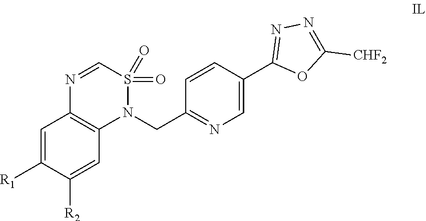

wherein R₁ and R₂ are defined as in formula I.

In another embodiment, this invention comprises HDAC6Is of formula IL, wherein R₁ and R₂ are independently selected from H, D, Cl, and F.

In another embodiment, this invention comprises HDAC6Is of formula Im

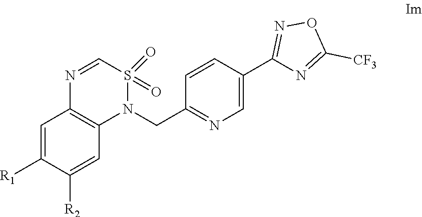

wherein R₁ and R₂ are defined as in formula I.

In another embodiment, this invention comprises HDAC6Is of formula Im, wherein R₁ and R₂ are independently selected from H, D, Cl, and F.

In another embodiment, this invention comprises HDAC6Is of formula In

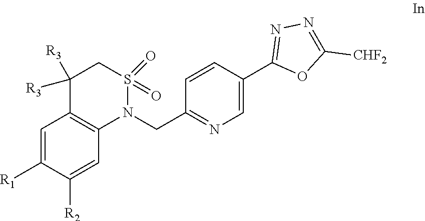

wherein R₁ and R₂ are defined as in claim 1, and both of R₃ are methyl, or one of R₃ is methyl and the other is H, or both of R₃ together are CH₂CH₂ or CH₂CH₂CH₂.

In another embodiment, this invention comprises HDAC6Is of formula In, wherein R₁ and R₂ are independently selected from H, D, Cl, and F.

In another embodiment, this invention comprises HDAC6Is of formula Io

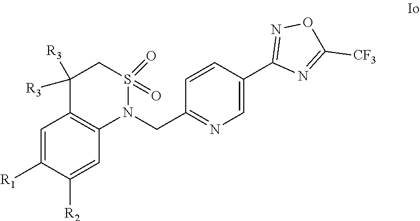

wherein R₁ and R₂ are defined as in claim 1, and both of R₁ are methyl, or one of R₃ is methyl and the other is H, or both of R₃ together are CH₂CH₂ or CH₂CH₂CH₂.

In another embodiment, this invention comprises HDAC6Is of formula Io, wherein R₁ and R₂ are independently selected from H, D, Cl, and F.

In another embodiment, this invention comprises HDAC6Is of formula Ip

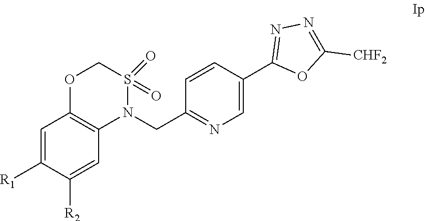

wherein R₁ and R₂ are defined as in formula I.

In another embodiment, this invention comprises HDAC6Is of formula Ip, wherein R₁ and R₂ are independently selected from H, D, Cl, and F.

In another embodiment, this invention comprises HDAC6Is of formula Iq

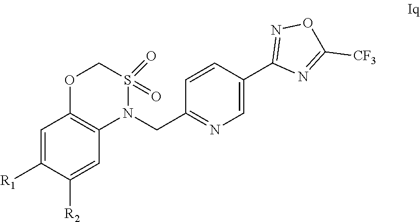

wherein R₁ and R₂ are defined as in formula I.

In another embodiment, this invention comprises HDAC6Is of formula Iq, wherein R₁ and R₂ are independently selected from H, D, Cl, and F.

In another embodiment, this invention comprises HDAC6Is of formula Ir

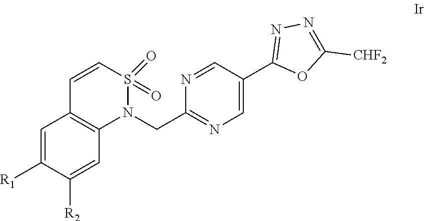

wherein R₁ and R₂ are defined as in formula I.

In another embodiment, this invention comprises HDAC6Is of formula Ir, wherein R₁ and R₂ are independently selected from H, D, Cl, and F.

In another embodiment, this invention comprises HDAC6Is of formula Is

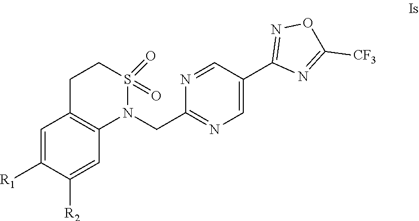

wherein R₁ and R₂ are defined as in formula I.

In another embodiment, this invention comprises HDAC6Is of formula It, wherein R₁ and R₂ are independently selected from H, D, Cl, and F.

In another embodiment, this invention comprises HDAC6Is of formula It

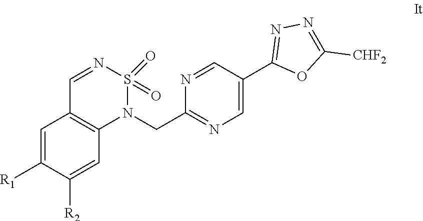

wherein R; and R₂ are defined as in formula I.

In another embodiment, this invention comprises HDAC6Is of formula It, wherein R₁ and R₂ are independently selected from H, D, Cl, and F.

In another embodiment, this invention comprises HDAC6Is of formula Iu

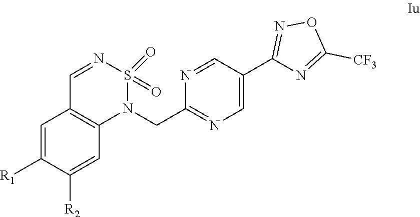

wherein R₁ and R₂ are defined as in formula I.

In another embodiment, this invention comprises HDAC6Is of formula Iu, wherein R₁ and R₂ are independently selected from H, D, Cl, and F.

In another embodiment, this invention comprises HDAC6Is of formula Iv

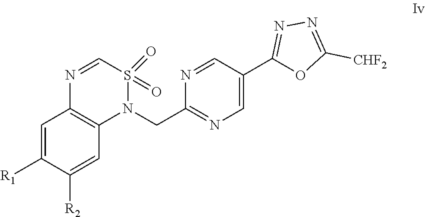

wherein R₁ and R₂ are defined as in formula I.

In another embodiment, this invention comprises HDAC6Is of formula Iv, wherein R₁ and R₂ are independently selected from H, D, Cl, and F.

In another embodiment, this invention comprises HDAC6Is of formula Iw

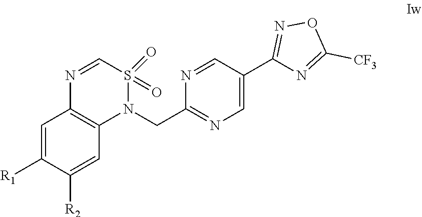

wherein R₁ and R₂ are defined as in formula I.

In another embodiment, this invention comprises HDAC6Is of formula Iw, wherein R₁ and R₂ are independently selected from H, D, Cl, and F.

In another embodiment, this invention comprises HDAC6Is of formula Ix

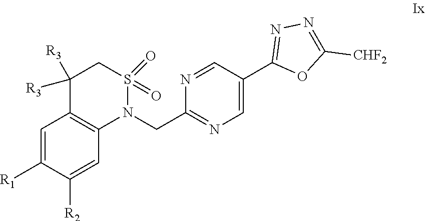

wherein R₁ and R₂ are defined as in claim 1, and both of R₁ are methyl, or one of R₃ is methyl and the other is H, or both of R₃ together are CH₂CH₂ or CH₂CH₂CH₂.

In another embodiment, this invention comprises HDAC6Is of formula Ix, wherein R₁ and R₂ are independently selected from H, D, Cl, and F.

In another embodiment, this invention comprises HDAC6Is of formula Iy

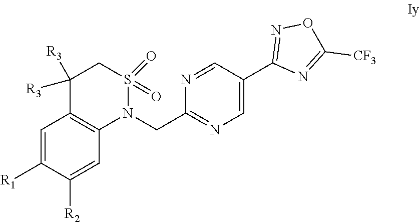

wherein R₁ and R₂ are defined as in claim 1, and both of R₃ are methyl, or one of R₃ is methyl and the other is H, or both of R₃ together are CH₂CH₂ or CH₂CH₂CH₂.

In another embodiment, this invention comprises HDAC6Is of formula Iy, wherein R₁ and R₂ are independently selected from H, D, Cl, and F.

In another embodiment, this invention comprises HDAC6Is of formula Iz

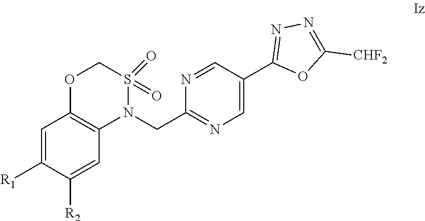

wherein R₁ and R₂ are defined as in formula I.

In another embodiment, this invention comprises HDAC6Is of formula Iz, wherein R₁ and R₂ are independently selected from H, D, Cl, and F.

In another embodiment, this invention comprises HDAC6Is of formula Iaa

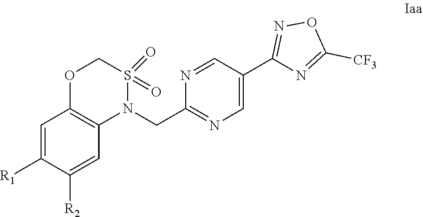

wherein R₁ and R₂ are defined as in formula I.

In another embodiment, this invention comprises HDAC6Is of formula Iaa, wherein R₁ and R₂ are independently selected from H, D, Cl, and F.

In another embodiment, this invention comprises HDAC6Is of formula Ibb

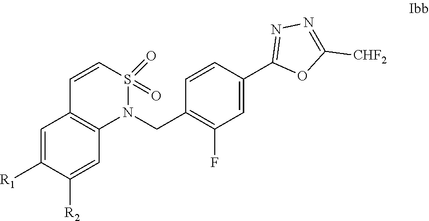

wherein R₁ and R₂ are defined as in formula I.

In another embodiment, this invention comprises HDAC6Is of formula Ibb, wherein R₁ and R₂ are independently selected from H, D, Cl, and F.

In another embodiment, this invention comprises HDAC6Is of formula Icc

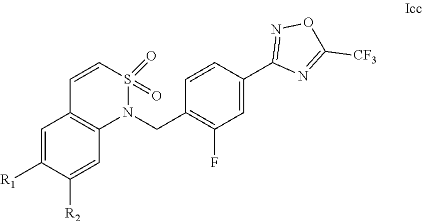

wherein R₁ and R₂ are defined as in formula I.

In another embodiment, this invention comprises HDAC6Is of formula Icc, wherein R₁ and R₂ are independently selected from H, D, Cl, and F.

In another embodiment, this invention comprises HDAC6Is of formula Idd

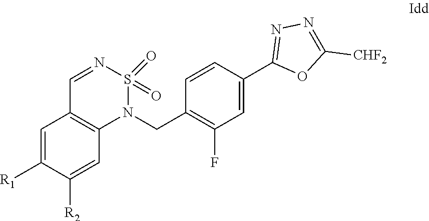

wherein R₁ and R₂ are defined as in formula I.

In another embodiment, this invention comprises HDAC6Is of formula Idd, wherein R₁ and R₂ are independently selected from H, D, Cl, and F.

In another embodiment, this invention comprises HDAC6Is of formula Iee

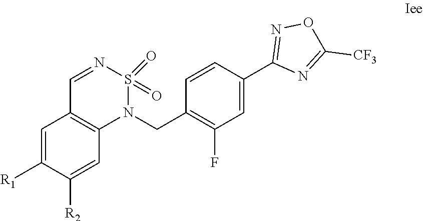

wherein R₁ and R₂ are defined as in formula I.

In another embodiment, this invention comprises HDAC6Is of formula Iee, wherein R₁ and R₂ are independently selected from H, D, Cl, and F.

In another embodiment, this invention comprises HDAC6Is of formula Iff

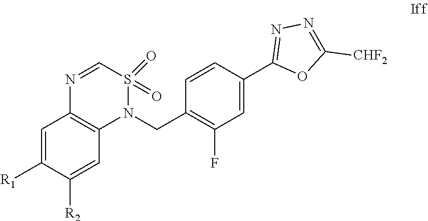

wherein R₁ and R₂ are defined as in formula I.

In another embodiment, this invention comprises HDAC6Is of formula Iff, wherein R₁ and R₂ are independently selected from H, D, Cl, and F.

In another embodiment, this invention comprises HDAC6Is of formula Igg

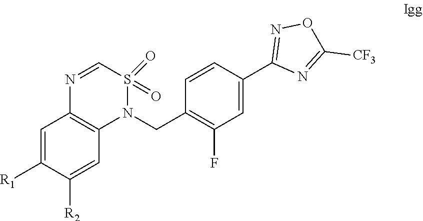

wherein R₁ and R₂ are defined as in formula I.

In another embodiment, this invention comprises HDAC6Is of formula Igg, wherein R₁ and R₂ are independently selected from H, D, Cl, and F.

In another embodiment, this invention comprises HDAC6Is of formula Ihh

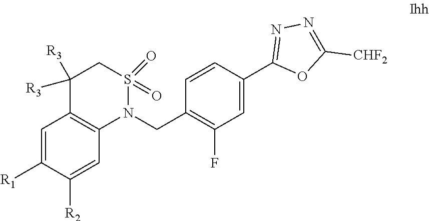

wherein R₁ and R₂ are defined as in claim 1, and both of R₃ are methyl, or one of R₃ is methyl and the other is H, or both of R₃ together are CH₂CH₂ or CH₂CH₂CH₂.

In another embodiment, this invention comprises HDAC6Is of formula Ihh, wherein R, and R₂ are independently selected from H, D, Cl, and F.

In another embodiment, this invention comprises HDAC6Is of formula Iii

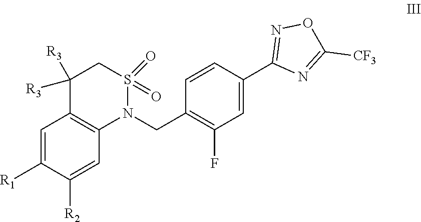

wherein R₁ and R₂ are defined as in claim 1, and both of R₃ are methyl, or one of R₃ is methyl and the other is H, or both of R₃ together are CH₂CH₂ or CH₂CH₂CH₂.

In another embodiment, this invention comprises HDAC6Is of formula Iii, wherein R₁ and R₂ are independently selected from H, D, Cl, and F.

In another embodiment, this invention comprises HDAC6Is of formula Ijj

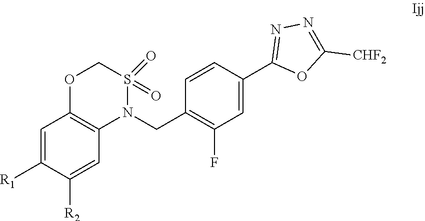

wherein R₁ and R₂ are defined as in formula I.

In another embodiment, this invention comprises HDAC6Is of formula Ijj, wherein R₁ and R₂ are independently selected from H, D, Cl, and F.

In another embodiment, this invention comprises HDAC6Is of formula Ikk

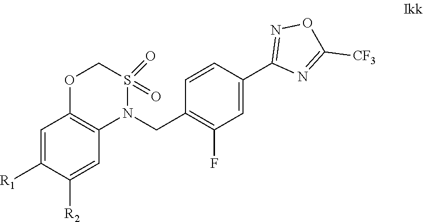

wherein R₁ and R₂ are defined as in formula I.

In another embodiment, this invention comprises HDAC6Is of formula Ikk, wherein R₁ and R₂ are independently selected from H, D, Cl, and F.

In another embodiment, this invention comprises HDAC6Is of formula ILL

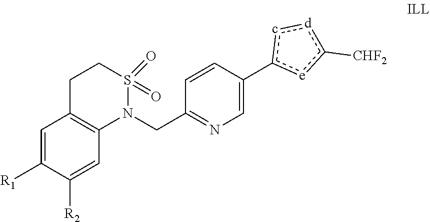

wherein
[symbol] represents a single or double bond; of the five bonds thus marked, two are double bonds and three are single bonds, together arranged in such manner as to give rise to a five-membered ring heteroaryl group;
one of c, d, and e is oxygen, and the remainder are nitrogen; or c is C(R_{f}), one of d and e is oxygen, and the other is nitrogen;

R_{f} is hydrogen, deuterium, fluorine, chlorine, methyl, difluoromethyl, trifluoromethyl, or cyano;

and R₁ and R₂ are defined as in Formula I.

In another embodiment, this invention comprises HDAC6Is of formula ILL, wherein R₁ and R₂ are independently selected from H, D, Cl, and F, c and d are nitrogen, and e is oxygen.

In another embodiment, this invention comprises HDAC6Is of formula Imm

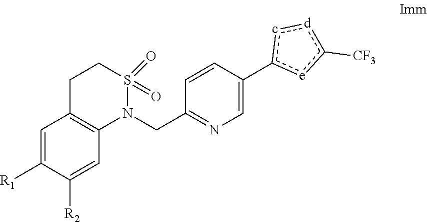

wherein
[symbol] represents a single or double bond; of the five bonds thus marked, two are double bonds and three are single bonds, together arranged in such manner as to give rise to a five-membered ring heteroaryl group;
one of c, d, and e is oxygen, and the remainder are nitrogen; or c is C(R_{f}), one of d and e is oxygen, and the other is nitrogen;

R_{f} is hydrogen, deuterium, fluorine, chlorine, methyl, difluoromethyl, trifluoromethyl, or cyano;

and R₁ and R₂ are defined as in Formula I.

In another embodiment, this invention comprises HDAC6Is of formula Imm, wherein R₁ and R₂ are independently selected from H, D, Cl, and F, c and d are nitrogen, and e is oxygen.

In another embodiment, this invention comprises HDAC6Is of formula Inn

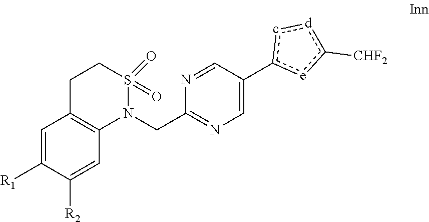

wherein
[symbol] represents a single or double bond; of the five bonds thus marked, two are double bonds and three are single bonds, together arranged in such manner as to give rise to a five-membered ring heteroaryl group;
one of c, d, and e is oxygen, and the remainder are nitrogen; or c is C(R_{f}), one of d and e is oxygen, and the other is nitrogen;

R_{f} is hydrogen, deuterium, fluorine, chlorine, methyl, difluoromethyl, trifluoromethyl, or cyano;

and R₁ and R₂ are defined as in Formula I.

In another embodiment, this invention comprises HDAC6Is of formula Inn, wherein R₁ and R₂ are independently selected from H, D, Cl, and F, c and dare nitrogen, and e is oxygen.

In another embodiment, this invention comprises HDAC6Is of formula Ioo

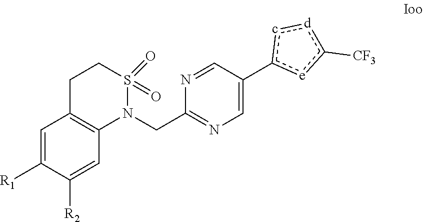

wherein
[symbol] represents a single or double bond; of the five bonds thus marked, two are double bonds and three are single bonds, together arranged in such manner as to give rise to a five-membered ring heteroaryl group;
one of c, d, and e is oxygen, and the remainder are nitrogen; or c is C(R_{f}), one of d and e is oxygen, and the other is nitrogen;

R_{f} is hydrogen, deuterium, fluorine, chlorine, methyl, difluoromethyl, trifluoromethyl, or cyano;

and R₁ and R₂ are defined as in Formula I.

In another embodiment, this invention comprises HDAC6Is of formula Ioo, wherein R₁ and R₂ are independently selected from H, D, Cl, and F, c and d are nitrogen, and e is oxygen.

In another embodiment, this invention comprises HDAC6Is of formula Ipp

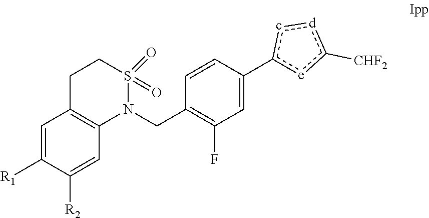

wherein
[symbol] represents a single or double bond; of the five bonds thus marked, two are double bonds and three are single bonds, together arranged in such manner as to give rise to a five-membered ring heteroaryl group;
one of c, d, and e is oxygen, and the remainder are nitrogen; or c is C(R_{f}), one of d and e is oxygen, and the other is nitrogen;

R_{f} is hydrogen, deuterium, fluorine, chlorine, methyl, difluoromethyl, trifluoromethyl, or cyano;

and R₁ and R₂ are defined as in Formula I.

In another embodiment, this invention comprises HDAC6Is of formula Ipp, wherein R₁ and R₂ are independently selected from H, D, Cl, and F, c and d are nitrogen, and e is oxygen.

In another embodiment, this invention comprises HDAC6Is of formula Iqq

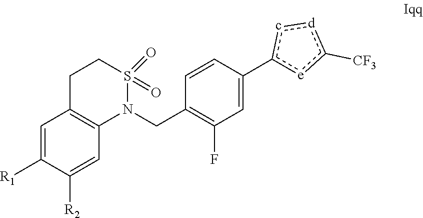

wherein
[symbol] represents a single or double bond; of the five bonds thus marked, two are double bonds and three are single bonds, together arranged in such manner as to give rise to a five-membered ring heteroaryl group;
one of c, d, and e is oxygen, and the remainder are nitrogen; or c is C(R_{f}), one of d and e is oxygen, and the other is nitrogen;

R_{f} is hydrogen, deuterium, fluorine, chlorine, methyl, difluoromethyl, trifluoromethyl, or cyano; and

R₁ and R₂ are defined as in Formula I.

In another embodiment, the invention comprises one of the compounds listed in Table 1.

TABLE 1
Examples of HDAC6Is containing a 1H-
benzo[c][1,2]thiazine-2,2-dioxide or related
moiety as the cap group.^{a}

| Ex- | |
|---|---|
| am- | |
| ple | |
| No. | Structure |
| 1 | 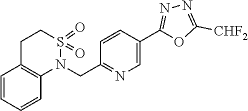 [SMILES: O=S1(=O)CCc2ccccc2N1Cc1ccc(-c2nnc(C(F)F)o2)cn1] |
| 2 | 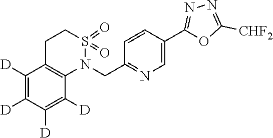 [SMILES: [2H]c1c([2H])c([2H])c2c(c1[2H])CCS(=O)(=O)N2Cc1ccc(-c2nnc(C(F)F)o2)cn1] |
| 3 | 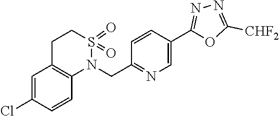 [SMILES: O=S1(=O)CCc2cc(Cl)ccc2N1Cc1ccc(-c2nnc(C(F)F)o2)cn1] |
| 4 | 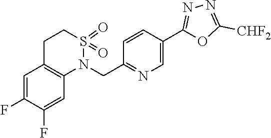 [SMILES: O=S1(=O)CCc2cc(F)c(F)cc2N1Cc1ccc(-c2nnc(C(F)F)o2)cn1] |
| 5 | 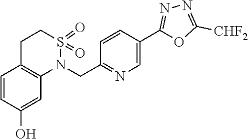 [SMILES: O=S1(=O)CCc2ccc(O)cc2N1Cc1ccc(-c2nnc(C(F)F)o2)cn1] |
| 6 | 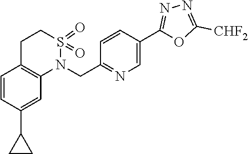 [SMILES: O=S1(=O)CCc2ccc(C3CC3)cc2N1Cc1ccc(-c2nnc(C(F)F)o2)cn1] |
| 7 | 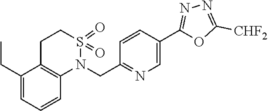 [SMILES: CCc1cccc2c1CCS(=O)(=O)N2Cc1ccc(-c2nnc(C(F)F)o2)cn1] |
| 8 | 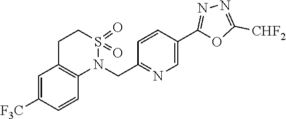 [SMILES: O=S1(=O)CCc2cc(C(F)(F)F)ccc2N1Cc1ccc(-c2nnc(C(F)F)o2)cn1] |
| 9 | 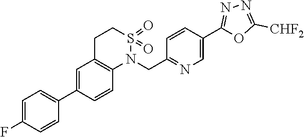 [SMILES: O=S1(=O)CCc2cc(-c3ccc(F)cc3)ccc2N1Cc1ccc(-c2nnc(C(F)F)o2)cn1] |
| 10 | 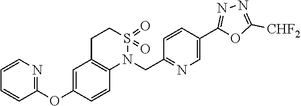 [SMILES: O=S1(=O)CCc2cc(Oc3ccccn3)ccc2N1Cc1ccc(-c2nnc(C(F)F)o2)cn1] |
| 11 | 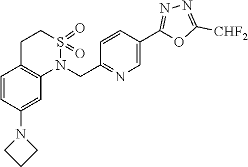 [SMILES: O=S1(=O)CCc2ccc(N3CCC3)cc2N1Cc1ccc(-c2nnc(C(F)F)o2)cn1] |
| 12 | 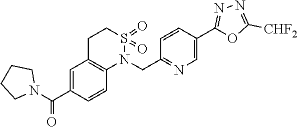 [SMILES: O=C(c1ccc2c(c1)CCS(=O)(=O)N2Cc1ccc(-c2nnc(C(F)F)o2)cn1)N1CCCC1] |
| 13 | 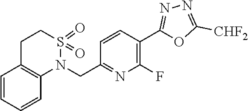 [SMILES: O=S1(=O)CCc2ccccc2N1Cc1ccc(-c2nnc(C(F)F)o2)c(F)n1] |
| 14 | 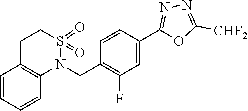 [SMILES: O=S1(=O)CCc2ccccc2N1Cc1ccc(-c2nnc(C(F)F)o2)cc1F] |
| 15 | 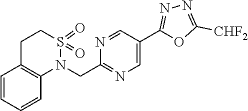 [SMILES: O=S1(=O)CCc2ccccc2N1Cc1ncc(-c2nnc(C(F)F)o2)cn1] |
| 16 | 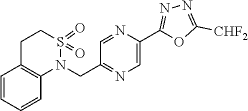 [SMILES: O=S1(=O)CCc2ccccc2N1Cc1cnc(-c2nnc(C(F)F)o2)cn1] |
| 17 | 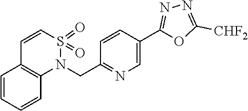 [SMILES: O=S1(=O)C=Cc2ccccc2N1Cc1ccc(-c2nnc(C(F)F)o2)cn1] |
| 18 | 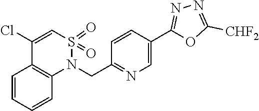 [SMILES: O=S1(=O)C=C(Cl)c2ccccc2N1Cc1ccc(-c2nnc(C(F)F)o2)cn1] |
| 19 | 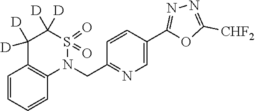 [SMILES: [2H]C1([2H])c2ccccc2N(Cc2ccc(-c3nnc(C(F)F)o3)cn2)S(=O)(=O)C1([2H])[2H]] |
| 20 | 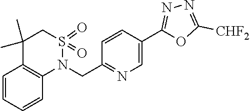 [SMILES: CC1(C)CS(=O)(=O)N(Cc2ccc(-c3nnc(C(F)F)o3)cn2)c2ccccc21] |
| 21 | 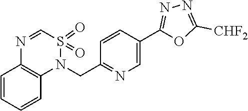 [SMILES: O=S1(=O)C=Nc2ccccc2N1Cc1ccc(-c2nnc(C(F)F)o2)cn1] |
| 22 | 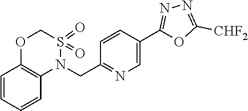 [SMILES: O=S1(=O)COc2ccccc2N1Cc1ccc(-c2nnc(C(F)F)o2)cn1] |
| 23 | 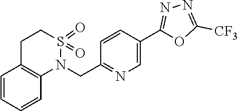 [SMILES: O=S1(=O)CCc2ccccc2N1Cc1ccc(-c2nnc(C(F)(F)F)o2)cn1] |
| 24 | 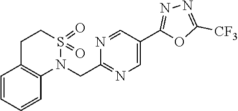 [SMILES: O=S1(=O)CCc2ccccc2N1Cc1ncc(-c2nnc(C(F)(F)F)o2)cn1] |
| 25 | 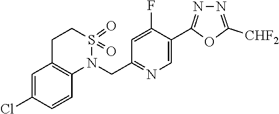 [SMILES: O=S1(=O)CCc2cc(Cl)ccc2N1Cc1cc(F)c(-c2nnc(C(F)F)o2)cn1] |
| 26 | 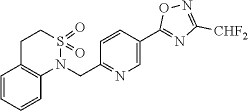 [SMILES: O=S1(=O)CCc2ccccc2N1Cc1ccc(-c2nc(C(F)F)no2)cn1] |
| 27 | 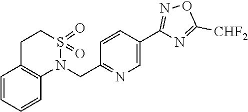 [SMILES: O=S1(=O)CCc2ccccc2N1Cc1ccc(-c2noc(C(F)F)n2)cn1] |
| 28 | 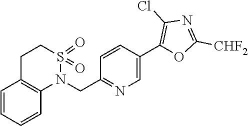 [SMILES: O=S1(=O)CCc2ccccc2N1Cc1ccc(-c2oc(C(F)F)nc2Cl)cn1] |
| 29 | 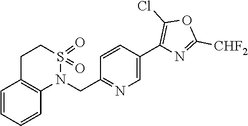 [SMILES: O=S1(=O)CCc2ccccc2N1Cc1ccc(-c2nc(C(F)F)oc2Cl)cn1] |
| 30 | 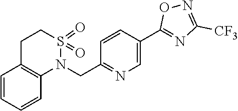 [SMILES: O=S1(=O)CCc2ccccc2N1Cc1ccc(-c2nc(C(F)(F)F)no2)cn1] |
| 31 | 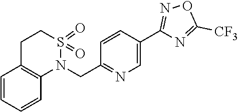 [SMILES: O=S1(=O)CCc2ccccc2N1Cc1ccc(-c2noc(C(F)(F)F)n2)cn1] |
| 32 | 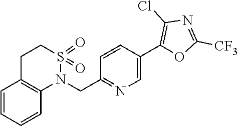 [SMILES: O=S1(=O)CCc2ccccc2N1Cc1ccc(-c2oc(C(F)(F)F)nc2Cl)cn1] |
| 33 | 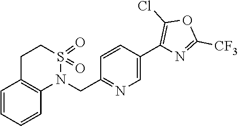 [SMILES: O=S1(=O)CCc2ccccc2N1Cc1ccc(-c2nc(C(F)(F)F)oc2Cl)cn1] |
| 34 | 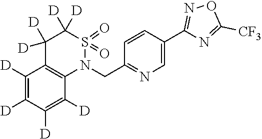 [SMILES: [2H]c1c([2H])c([2H])c2c(c1[2H])N(Cc1ccc(-c3noc(C(F)(F)F)n3)cn1)S(=O)(=O)C([2H])([2H])C2([2H])[2H]] |
| 35 | 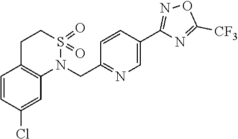 [SMILES: O=S1(=O)CCc2ccc(Cl)cc2N1Cc1ccc(-c2noc(C(F)(F)F)n2)cn1] |
| 36 | 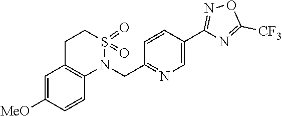 [SMILES: COc1ccc2c(c1)CCS(=O)(=O)N2Cc1ccc(-c2noc(C(F)(F)F)n2)cn1] |
| 37 | 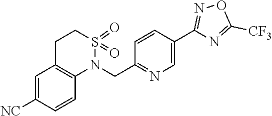 [SMILES: N#Cc1ccc2c(c1)CCS(=O)(=O)N2Cc1ccc(-c2noc(C(F)(F)F)n2)cn1] |
| 38 | 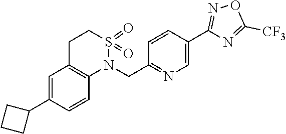 [SMILES: O=S1(=O)CCc2cc(C3CCC3)ccc2N1Cc1ccc(-c2noc(C(F)(F)F)n2)cn1] |
| 39 | 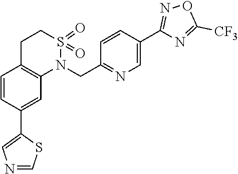 [SMILES: O=S1(=O)CCc2ccc(-c3cncs3)cc2N1Cc1ccc(-c2noc(C(F)(F)F)n2)cn1] |
| 40 | 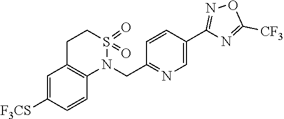 |
| 41 | 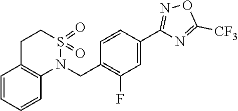 [SMILES: O=S1(=O)CCc2ccccc2N1Cc1ccc(-c2noc(C(F)(F)F)n2)cc1F] |
| 42 | 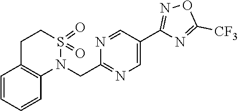 [SMILES: O=S1(=O)CCc2ccccc2N1Cc1ncc(-c2noc(C(F)(F)F)n2)cn1] |
| 43 | 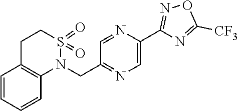 [SMILES: O=S1(=O)CCc2ccccc2N1Cc1cnc(-c2noc(C(F)(F)F)n2)cn1] |
| 44 | 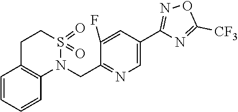 [SMILES: O=S1(=O)CCc2ccccc2N1Cc1ncc(-c2noc(C(F)(F)F)n2)cc1F] |
| 45 | 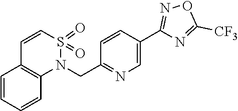 [SMILES: O=S1(=O)C=Cc2ccccc2N1Cc1ccc(-c2noc(C(F)(F)F)n2)cn1] |
| 46 | 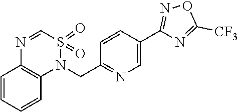 [SMILES: O=S1(=O)C=Nc2ccccc2N1Cc1ccc(-c2noc(C(F)(F)F)n2)cn1] |
| 47 | 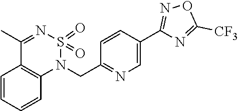 [SMILES: CC1=NS(=O)(=O)N(Cc2ccc(-c3noc(C(F)(F)F)n3)cn2)c2ccccc21] |
| 48 | 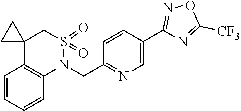 [SMILES: O=S1(=O)CC2(CC2)c2ccccc2N1Cc1ccc(-c2noc(C(F)(F)F)n2)cn1] |
| 49 | 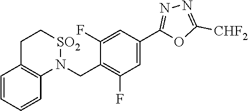 [SMILES: O=S1(=O)CCc2ccccc2N1Cc1c(F)cc(-c2nnc(C(F)F)o2)cc1F] |
| 50 | 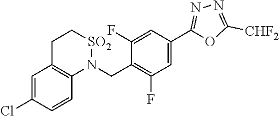 [SMILES: O=S1(=O)CCc2cc(Cl)ccc2N1Cc1c(F)cc(-c2nnc(C(F)F)o2)cc1F] |
| 51 | 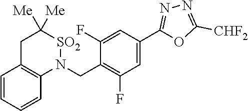 [SMILES: CC1(C)Cc2ccccc2N(Cc2c(F)cc(-c3nnc(C(F)F)o3)cc2F)S1(=O)=O] |

Additionally, salts, prodrugs, hydrates, isotopically labeled and fluorescently labeled derivatives, and any other therapeutically or diagnostically relevant derivations of the present HDAC6Is also are included in the present invention and can be used in the methods disclosed herein. The present invention further includes all possible stereoisomers and geometric isomers of the present compounds. The present invention includes both racemic compounds and optically active isomers. When a present HDAC6I is desired as a single enantiomer, it can be obtained either by resolution of the final product or by stereospecific synthesis from either isomerically pure starting material or use of a chiral auxiliary reagent, for example, see Ma, Z. et al., Tetrahedron: Asymmetry, 1997, 8, 883-888. Resolution of the final product, an intermediate, or a starting material can be achieved by any suitable method known in the art. Additionally, in situations where tautomers of a present compound are possible, the present invention is intended to include all tautomeric forms of the compounds.

Deuterium-labeled compounds of the present invention are of interest due to the observation that C-D bonds are less readily broken than C—H bonds, and therefore metabolic degradation involving attack on these bonds is slowed down upon replacement of H by D, resulting in an increased half-life of the drug substance (see, for example: Halford, B. Chem. Eng. News 2016, 94(27), 32-36). Preferred C—H bonds to be replaced with C-D bonds are aliphatic C—H bonds adjacent to an amine nitrogen atom; aliphatic C—H bonds adjacent to the oxygen atom of an alkoxy substituent; and aromatic C—H bonds, which may be replaced fully or in part with C-D bonds.

Lactams and amides can be reduced to deuterated amines containing the moiety CD₂ adjacent to their amine nitrogen atom, with commercially available deuterated versions of reducing agents commonly employed for their reduction to amines, such as LiAlD₄ and BD₃-THF. Alternatively, alkylation of a nitrogen atom with a deuterated alkyl group may be effected by treatment with the corresponding deuterated alkyl halide in the presence of a base, or by reductive alkylation with the corresponding deuterated aldehyde [containing the moiety C(D)O] using, for example, NaBD(OAc)₃ in AcOD as the reducing agent. When a methoxy substituent is present on one of the aromatic rings of the compounds of the invention, its trideuteriomethoxy analog may be obtained by alkylation of the corresponding phenol with CD₃I or (CD₃)₂SO₄ in the presence of a suitable base, such as K₂CO₃ or NaH. Similarly, alkylation of the same phenol with RCD₂Br or RCD₂I and a base produces 1,1-dideuterioalkyl ethers. Compounds containing deuterium as a substituent on one or more aromatic rings may be obtained by performing the synthesis from perdeuterated starting materials, or by selectively installing deuterium atoms through reduction of an aryl bromide or iodide with tri-n-butyltin deuteride in the presence of a transition metal catalyst, such as a palladium compound.

Prodrugs of the present compounds also are included in the present invention. It is well established that a prodrug approach, wherein a compound is derivatized into a form suitable for formulation and/or administration, then released as a drug in vivo, has been successfully employed to transiently (e.g., bioreversibly) alter the physicochemical properties of the compound (see, H. Bundgaard, Ed., “Design of Prodrugs,” Elsevier, Amsterdam, (1985); R B. Silverman, “The Organic Chemistry of Drug Design and Drug Action,” Academic Press, San Diego, chapter 8, (1992); K. M. Hillgren et al., Med. Res. Rev., 15, 83 (1995)).

Compounds of the invention can exist as salts. Pharmaceutically acceptable salts of the present HDAC6Is often are preferred in the methods of the invention. As used herein, the term “pharmaceutically acceptable salts” refers to salts or zwitterionic forms of the present compounds. Salts of the present compounds can be prepared during the final isolation and purification of the compounds or separately by reacting the compound with an acid having a suitable anion. The pharmaceutically acceptable salts of the present compounds can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, tartaric, and citric. Examples of salts of compounds of the invention include, but are not limited to, the hydrochloride, hydrobromide, hydroiodide, sulfate, bisulfate, 2-hydroxyethanesulfonate, phosphate, hydrogen phosphate, acetate, adipate, alginate, aspartate, benzoate, bisulfate, butyrate, camphorate, camphorsulfonate, digluconate, glycerolphosphate, hemisulfate, heptanoate, hexanoate, formate, succinate, fumarate, maleate, ascorbate, isethionate, salicylate, methanesulfonate, mesitylenesulfonate, 1-naphthalenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, undecanoate, lactate, citrate, tartrate, gluconate, ethanedisulfonate, benzenesulfonate, and p-toluenesulfonate salts. In addition, available amino groups present in the compounds of the invention can be quaternized with methyl, ethyl, propyl, and butyl chlorides, bromides, and iodides; dimethyl, diethyl, dibutyl, and diamyl sulfates; decyl, lauryl, myristyl, and stearyl chlorides, bromides, and iodides; and benzyl and phenethyl bromides. In light of the foregoing, any reference to compounds of the present invention appearing herein is intended to include the present compounds as well as pharmaceutically acceptable salts, hydrates, or prodrugs thereof.

The present compounds also can be conjugated or linked to auxiliary moieties that promote a beneficial property of the compound in a method of therapeutic use. Such conjugates can enhance delivery of the compounds to a particular anatomical site or region of interest (e.g., a tumor), enable sustained therapeutic concentrations of the compounds in target cells, alter pharmacokinetic and pharmacodynamic properties of the compounds, and/or improve the therapeutic index or safety profile of the compounds. Suitable auxiliary moieties include, for example, amino acids, oligopeptides, or polypeptides, e.g., antibodies, such as monoclonal antibodies and other engineered antibodies; and natural or synthetic ligands to receptors in target cells or tissues. Other suitable auxiliaries include fatty acid or lipid moieties that promote biodistribution and/or uptake of the compound by target cells (see, e.g., Bradley et al., Clin. Cancer Res. (2001) 7:3229).

Compounds of the present invention inhibit HDAC6 and are useful in the treatment of a variety of diseases and conditions. In particular, the present HDAC6Is are used in methods of treating a disease or condition wherein inhibition of HDAC6 provides a benefit, for example, cancers, neurological diseases, neurodegenerative conditions, peripheral neuropathies, autoimmune diseases, inflammatory diseases and conditions, stroke, hypertension, traumatic brain injury, autism, and malaria. The methods comprise administering a therapeutically effective amount of a present HDAC6I to an individual in need thereof.

The present methods also encompass administering a second therapeutic agent to the individual in addition to a present HDAC6I. The second therapeutic agent is selected from agents, such as drugs and adjuvants, known as useful in treating the disease or condition afflicting the individual, e.g., a chemotherapeutic agent and/or radiation known as useful in treating a particular cancer.

The present compounds have been evaluated for their activity at HDAC6 and their selectivity for HDAC6 compared to other HDACs. It previously was shown that selective HDAC6 inhibitors may find application in a variety of disease states including, but not limited to, arthritis, autoimmune disorders, inflammatory disorders, cancer, neurological diseases such as Rett syndrome, peripheral neuropathies such as CMT, stroke, hypertension, and diseases in which oxidative stress is a causative factor or a result thereof. It also was shown that selective HDAC6 inhibitors, when administered in combination with rapamycin, prolonged the lifespan of mice with kidney xenografts. This model was used to evaluate the immunosuppressant properties of the present compounds and serve as a model of transplant rejection. Furthermore, it was previously shown that selective HDAC6 inhibitors confer neuroprotection in rat primary cortical neuron models of oxidative stress. These studies identified selective HDAC6 inhibitors as non-toxic neuroprotective agents. The present compounds behave in a similar manner because they also are selective HDAC6 agents. The present compounds demonstrate a ligand efficiency that renders them more drug-like in their physiochemical properties. The present compounds therefore are pharmaceutical candidates and research tools to identify the specific functions of HDAC6.

Thus, in one embodiment, the present invention relates to a method of treating an individual suffering from a disease or condition wherein inhibition of HDAC6 provides a benefit comprising administering a therapeutically effective amount of a claimed HDAC6I compound to an individual in need thereof.

The methods of the present invention can be accomplished by administering one of the HDAC6Is of the present invention as the neat compound or as a pharmaceutical composition. Administration of a pharmaceutical composition, or a neat HDAC6I of the present invention, can be performed during or after the onset of the disease or condition of interest. Typically, the pharmaceutical compositions are sterile, and contain no toxic, carcinogenic, or mutagenic compounds that would cause an adverse reaction when administered.

In some embodiments, a present HDAC6I may be administered in conjunction with a second therapeutic agent useful in the treatment of a disease or condition wherein inhibition of HDAC6 provides a benefit. The second therapeutic agent is different from the present HDA6CIs. A present HDAC6I and the second therapeutic agent can be administered simultaneously or sequentially. In addition, a present HDAC6I and second therapeutic agent can be administered from a single composition or two separate compositions. A present HDAC6I and the second therapeutic agent can be administered simultaneously or sequentially to achieve the desired effect.

The second therapeutic agent is administered in an amount to provide its desired therapeutic effect. The effective dosage range for each second therapeutic agent is known in the art, and the second therapeutic agent is administered to an individual in need thereof within such established ranges.

The present invention therefore is directed to compositions and methods of using such compounds in treating diseases or conditions wherein inhibition of HDAC6 provides a benefit. The present invention also is directed to pharmaceutical compositions comprising a present HDAC6I and an optional second therapeutic agent useful in the treatment of diseases and conditions wherein inhibition of HDAC6 provides a benefit. Further provided are kits comprising a present HDAC6I and, optionally, a second therapeutic agent useful in the treatment of diseases and conditions wherein inhibition of HDAC6 provides a benefit, packaged separately or together, and an insert having instructions for using these active agents.

A present HDAC6I and the second therapeutic agent can be administered together as a single-unit dose or separately as multi-unit doses, wherein the present HDAC6I is administered before the second therapeutic agent or vice versa. One or more dose of a present HDAC6I and/or one or more dose of the second therapeutic agent can be administered. The present HDAC6Is therefore can be used in conjunction with one or more second therapeutic agents, for example, but not limited to, anticancer agents.

Within the meaning of the present invention, the term “disease” or “condition” denotes disturbances and/or anomalies that as a rule are regarded as being pathological conditions or functions, and that can manifest themselves in the form of particular signs, symptoms, and/or malfunctions. As demonstrated below, a present HDAC6I is a potent inhibitor of HDAC6 and can be used in treating diseases and conditions wherein inhibition of HDAC6 provides a benefit, for example, cancer, a neurological disease, a neurodegenerative condition, traumatic brain injury, stroke, an inflammation, an autoimmune disease, and autism.

In one embodiment, the present invention provides methods for treating cancer, including but not limited to killing a cancer cell or neoplastic cell; inhibiting the growth of a cancer cell or neoplastic cell; inhibiting the replication of a cancer cell or neoplastic cell; or ameliorating a symptom thereof, said methods comprising administering to a subject in need thereof an amount of a present HDAC6I or a pharmaceutically acceptable salt thereof sufficient to treat the cancer. Additionally, it is noted that the selective HDAC6I may be able to facilitate the killing of cancer cells through reactivation of the immune system by mechanisms relating to the PDI receptor. A present HDAC6I can be used as the sole anticancer agent, or in combination with another anticancer treatment, e.g., radiation, chemotherapy, and surgery.

In another embodiment, the invention provides a method for increasing the sensitivity of a cancer cell to the cytotoxic effects of radiotherapy and/or chemotherapy comprising contacting the cell with a present HDAC6I or a pharmaceutically acceptable salt thereof in an amount sufficient to increase the sensitivity of the cell to the cytotoxic effects of radiotherapy and/or chemotherapy. The HDAC6is of the present invention may thus be combined with an antibody directed toward PD-1 and/or PD-L1 in order to achieve higher efficacy.

In a further embodiment, the present invention provides a method for treating cancer comprising: (a) administering to an individual in need thereof an amount of a present HDAC6I compound; and (b) administering to the individual an amount of radiotherapy, chemotherapy, or both. The amounts administered are each effective to treat cancer. In another embodiment, the amounts are together effective to treat cancer.

This combination therapy of the invention can be used accordingly in a variety of settings for the treatment of various cancers. In a specific embodiment, the individual in need of treatment has previously undergone treatment for cancer. Such previous treatments include, but are not limited to, prior chemotherapy, radiotherapy, surgery, or immunotherapy, such as cancer vaccines.

In another embodiment, the cancer being treated is a cancer which has demonstrated sensitivity to radiotherapy and/or chemotherapy or is known to be responsive to radiotherapy and/or chemotherapy. Such cancers include, but are not limited to, non-Hodgkin's lymphoma, Hodgkin's disease, Ewing's sarcoma, testicular cancer, prostate cancer, ovarian cancer, bladder cancer, larynx cancer, cervical cancer, nasopharynx cancer, breast cancer, colon cancer, pancreatic cancer, head and neck cancer, esophageal cancer, rectal cancer, small-cell lung cancer, non-small cell lung cancer, brain tumors, or other CNS neoplasms.

In still another embodiment, the cancer being treated has demonstrated resistance to radiotherapy and/or chemotherapy or is known to be refractory to radiotherapy and/or chemotherapy. A cancer is refractory to a therapy when at least some significant portion of the cancer cells are not killed or their cell division is not arrested in response to therapy. Such a determination can be made either in vivo or in vitro by any method known in the art for assaying the effectiveness of treatment on cancer cells, using the art-accepted meanings of “refractory” in such a context. In a specific embodiment, a cancer is refractory where the number of cancer cells has not been significantly reduced or has increased.

Other cancers that can be treated with the compounds and methods of the invention include, but are not limited to, cancers and metastases, such as brain cancers (glioblastomas) and melanomas, as well as other common tumors.

In a specific embodiment, leukoplakia, a benign-appearing hyperplastic or dysplastic lesion of the epithelium, and Bowen's disease, a carcinoma in situ, are pre-neoplastic lesions indicative of the desirability of prophylactic intervention.

In another embodiment, fibrocystic disease (cystic hyperplasia, mammary dysplasia, particularly adenosis (benign epithelial hyperplasia)), is indicative of the desirability of prophylactic intervention.

The prophylactic use of the compounds and methods of the present invention is also indicated in some viral infections that may lead to cancer. For example, human papilloma virus can lead to cervical cancer (see, e.g., Hernandez-Avila et al., Archives of Medical Research (1997) 28:265-271), Epstein-Barr virus (EBV) can lead to lymphoma (see, e.g., Herrmann et al., J. Pathol. (2003) 199(2):140-5), hepatitis B or C virus can lead to liver carcinoma (see, e.g., El-Serag, J. Clin. Gastroenterol. (2002) 35(5 Suppl 2):S72-8), human T cell leukemia virus (HTLV)—I can lead to T-cell leukemia (see e.g., Mortreux et al., Leukemia (2003) 17(1):26-38), human herpesvirus-8 infection can lead to Kaposi's sarcoma (see, e.g., Kadow et al., Curr. Opin. Investig. Drugs (2002) 3(11):1574-9), and Human Immunodeficiency Virus (HIV) infection contribute to cancer development as a consequence of immunodeficiency (see, e.g., Dal Maso et al., Lancet Oncol. (2003) 4(2):110-9).

In other embodiments, a subject exhibiting one or more of the following predisposing factors for malignancy can be treated by administration of the present HDAC6Is and methods of the invention: a chromosomal translocation associated with a malignancy (e.g., the Philadelphia chromosome for chronic myelogenous leukemia, t(14,18) for follicular lymphoma, etc.), familial polyposis or Gardner's syndrome (possible forerunners of colon cancer), benign monoclonal gammopathy (a possible forerunner of multiple myeloma), a first degree kinship with persons having a cancer or procancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome (see Robbins and Angell, 1976, Basic Pathology, 2nd Ed., W.B. Saunders Co., Philadelphia, pp. 112-113 etc.), and exposure to carcinogens (e.g., smoking, and inhalation of or contacting with certain chemicals).

In another specific embodiment, the present HDAC6Is and methods of the invention are administered to a human subject to prevent progression of breast, colon, ovarian, or cervical cancer.

In one embodiment, the invention provides methods for treating cancer comprising (a) administering to an individual in need thereof an amount of a present HDAC6I; and (b) administering to the individual one or more additional anticancer treatment modalities including, but not limited to, radiotherapy, chemotherapy including certain antibodies such as those directed toward PD-1 and PD-L1, surgery, or immunotherapy, such as a cancer vaccine. In one embodiment, the administering of step (a) is prior to the administering of step (b). In another embodiment, the administering of step (a) is subsequent to the administering of step (b). In still another embodiment, the administering of step (a) is concurrent with the administering of step (b).

In one embodiment, the additional anticancer treatment modality is radiotherapy and/or chemotherapy. In another embodiment, the additional anticancer treatment modality is surgery.

In still another embodiment, the additional anticancer treatment modality is immunotherapy, such as cancer vaccines.

In one embodiment, a present HDAC6I or a pharmaceutically acceptable salt thereof is administered adjunctively with the additional anticancer treatment modality.

In a preferred embodiment, the additional anticancer treatment modality is radiotherapy. In the methods of the present invention, any radiotherapy protocol can be used depending upon the type of cancer to be treated. Embodiments of the present invention employ electromagnetic radiation of various wavelengths: gamma-radiation (10⁻²⁰ to 10⁻¹³ m), X-ray radiation (10⁻¹² to 10⁻⁹ m), ultraviolet light (10 nm to 400 nm), visible light (400 nm to 700 nm), infrared radiation (700 nm to 1 mm), and microwave radiation (1 mm to 30 cm).

For example, but not by way of limitation, X-ray radiation can be administered; in particular, high-energy megavoltage (radiation of greater that 1 MeV energy) can be used for deep tumors, and electron beam and orthovoltage X-ray radiation can be used for skin cancers. Gamma-ray emitting radioisotopes, such as radioactive isotopes of radium, cobalt, and other elements, can also be administered. Illustrative radiotherapy protocols useful in the present invention include, but are not limited to, stereotactic methods where multiple sources of low dose radiation are simultaneously focused into a tissue volume from multiple angles; “internal radiotherapy,” such as brachytherapy, interstitial irradiation, and intracavitary irradiation, which involves the placement of radioactive implants directly in a tumor or other target tissue: intraoperative irradiation, in which a large dose of external radiation is directed at the target tissue which is exposed during surgery; and particle beam radiotherapy, which involves the use of fast-moving subatomic particles to treat localized cancers.

Many cancer treatment protocols currently employ radiosensitizers activated by electromagnetic radiation, e.g., X-rays. Examples of X-ray-activated radiosensitizers include, but are not limited to, metronidazole, misonidazole, desmethylmisonidazole, pimonidazole, etanidazole, nimorazole, mitomycin C, RSU 1069, SR 4233, EO9, RB 6145, nicotinamide, 5-bromodeoxyuridine (BUdR), 5-iododeoxyuridine (IUdR), bromodeoxycytidine, fluorodeoxyuridine (FUdR), hydroxyurea, cis-platin, and therapeutically effective analogs and derivatives of the same.

Photodynamic therapy (PDT) of cancers employs visible light as the radiation activator of the sensitizing agent. Examples of photodynamic radiosensitizers include the following, but are not limited to: hematoporphyrin derivatives, PHOTOFRIN®, benzoporphyrin derivatives, NPe6, tin etioporphyrin (SnET2), pheoborbide-a, bacteriochlorophyll-a, naphthalocyanines, phthalocyanines, zinc phthalocyanine, and therapeutically effective analogs and derivatives of the same.

Radiosensitizers can be administered in conjunction with a therapeutically effective amount of one or more compounds in addition to a present HDAC6I, such compounds including, but not limited to, compounds that promote the incorporation of radiosensitizers to the target cells, compounds that control the flow of therapeutics, nutrients, and/or oxygen to the target cells, chemotherapeutic agents that act on the tumor with or without additional radiation, or other therapeutically effective compounds for treating cancer or other diseases. Examples of additional therapeutic agents that can be used in conjunction with radiosensitizers include, but are not limited to, 5-fluorouracil (5-FU), leucovorin, oxygen, carbogen, red cell transfusions, perfluorocarbons (e.g., FLUOSOLW®-DA), 2,3-DPG, BW12C, calcium channel blockers, pentoxifylline, antiangiogenesis compounds, hydralazine, and L-BSO.

In an embodiment, a present HDAC6I or a pharmaceutically acceptable salt thereof is administered prior to the administration of radiotherapy and/or chemotherapy.

In another embodiment, a present HDAC6I or a pharmaceutically acceptable salt thereof is administered adjunctively with radiotherapy and/or chemotherapy.

A present HDAC6I and additional treatment modalities can act additively or synergistically (i.e., the combination of a present HDAC6I or a pharmaceutically acceptable salt thereof, and an additional anticancer treatment modality is more effective than their additive effects when each are administered alone). A synergistic combination permits the use of lower dosages of a present HDAC6I and/or the additional treatment modality and/or less frequent administration of a present HDAC6I and/or additional treatment modality to a subject with cancer. The ability to utilize lower dosages of a present HDAC6I and/or an additional treatment modality and/or to administer a compound of the invention and the additional treatment modality less frequently can reduce the toxicity associated with the administration without reducing the efficacy of a present HDAC6I and/or the additional treatment modality in the treatment of cancer. In addition, a synergistic effect can result in the improved efficacy of the treatment of cancer and/or the reduction of adverse or unwanted side effects associated with the administration of a present HDAC6I and/or an additional anticancer treatment modality as monotherapy.

In one embodiment, the present HDAC6Is may act synergistically with radiotherapy when administered in doses typically employed when such HDAC6Is are used alone for the treatment of cancer. In another embodiment, the present HDAC6Is may act synergistically with radiotherapy when administered in doses that are less than doses typically employed when such HDAC6Is are used as monotherapy for the treatment of cancer.

In one embodiment, radiotherapy may act synergistically with a present HDAC6I when administered in doses typically employed when radiotherapy is used as monotherapy for the treatment of cancer. In another embodiment, radiotherapy may act synergistically with a compound of the invention when administered in doses that are less than doses typically employed when radiotherapy is used as monotherapy for the treatment of cancer.

The effectiveness of the HDAC6Is as HDAC6 inhibitors for sensitizing cancer cells to the effect of radiotherapy can be determined by the in vitro and/or in vivo determination of post-treatment survival using techniques known in the art. In one embodiment, for in vitro determinations, exponentially growing cells can be exposed to known doses of radiation, and the survival of the cells monitored. Irradiated cells are plated and cultured for about 14 to about 21 days, and the colonies are stained. The surviving fraction is the number of colonies divided by the plating efficiency of unirradiated cells. Graphing the surviving fraction on a log scale versus the absorbed dose on a linear scale generates a survival curve. Survival curves generally show an exponential decrease in the fraction of surviving cells at higher radiation doses after an initial shoulder region in which the dose is sublethal. A similar protocol can be used for chemical agents when used in the combination therapies of the invention.

Inherent radiosensitivity of tumor cells and environmental influences, such as hypoxia and host immunity, can be further assessed by in vivo studies. The growth delay assay is commonly used. This assay measures the time interval required for a tumor exposed to radiation to regrow to a specified volume. The dose required to control about 50% of tumors is determined by the TCD₅₀ assay.

In vivo assay systems typically use transplantable solid tumor systems in experimental subjects. Radiation survival parameters for normal tissues as well as for tumors can be assayed using in vivo methods known in the art.

The present invention provides methods of treating cancers comprising the administration of an effective amount of a present HDAC6I in conjunction with recognized methods of surgery, radiotherapy, and chemotherapies, including, for example, chemical-based mimics of radiotherapy whereby a synergistic enhancement of the effectiveness of the recognized therapy is achieved. The effectiveness of a treatment can be measured in clinical studies or in model systems, such as a tumor model in mice, or cell culture sensitivity assays.

The present invention provides combination therapies that result in improved effectiveness and/or reduced toxicity. Accordingly, in one aspect, the invention relates to the use of the present HDAC6Is as radiosensitizers in conjunction with radiotherapy.

When the combination therapy of the invention comprises administering a present HDAC6I with one or more additional anticancer agents, the present HDAC6I and the additional anticancer agents can be administered concurrently or sequentially to an individual. The agents can also be cyclically administered. Cycling therapy involves the administration of one or more anticancer agents for a period of time, followed by the administration of one or more different anticancer agents for a period of time and repeating this sequential administration, i.e., the cycle, in order to reduce the development of resistance to one or more of the anticancer agents of being administered, to avoid or reduce the side effects of one or more of the anticancer agents being administered, and/or to improve the efficacy of the treatment.

An additional anticancer agent may be administered over a series of sessions; any one or a combination of the additional anticancer agents listed below may be administered.

The present invention includes methods for treating cancer comprising administering to an individual in need thereof a present HDAC6I and one or more additional anticancer agents or pharmaceutically acceptable salts thereof. A present HDAC6I and the additional anticancer agent can act additively or synergistically. Suitable anticancer agents include, but are not limited to, gemcitabine, capecitabine, methotrexate, taxol, taxotere, and the like.

Additionally, the invention provides methods of treatment of cancer using the present HDAC6Is as an alternative to chemotherapy alone or radiotherapy alone where the chemotherapy or the radiotherapy has proven or can prove too toxic, e.g., results in unacceptable or unbearable side effects, for the subject being treated. The individual being treated can, optionally, be treated with another anticancer treatment modality such as chemotherapy, surgery, or immunotherapy, depending on which treatment is found to be acceptable or bearable.

The present HDAC6Is can also be used in an in vitro or ex vivo fashion, such as for the treatment of certain cancers, including, but not limited to leukemias and lymphomas, such treatment involving autologous stem cell transplants. This can involve a multi-step process in which the subject's autologous hematopoietic stem cells are harvested and purged of all cancer cells, the subject is then administered an amount of a present HDAC6I effective to eradicate the subject's remaining bone-marrow cell population, then the stem cell graft is infused back into the subject. Supportive care then is provided while bone marrow function is restored and the subject recovers.

The present methods for treating cancer can further comprise the administration of a present HDAC6I and an additional therapeutic agent or pharmaceutically acceptable salts or hydrates thereof. In one embodiment, a composition comprising a present HDAC6I is administered concurrently with the administration of one or more additional therapeutic agent(s), which may be part of the same composition or in a different composition from that comprising the present HDAC6I. In another embodiment, a present HDAC6I is administered prior to or subsequent to administration of another therapeutic agent(s).

In the present methods for treating cancer the other therapeutic agent may be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinols, thiethylperazine, thioproperazine, and tropisetron.

In one embodiment, the antiemetic agent is granisetron or ondansetron. In another embodiment, the other therapeutic agent may be a hematopoietic colony stimulating factor. Suitable hematopoietic colony stimulating factors include, but are not limited to, filgrastim, sargramostim, molgramostim, and epoietin alfa.

In still another embodiment, the other therapeutic agent may be an opioid or nonopioid analgesic agent. Suitable opioid analgesic agents include, but are not limited to, morphine, heroin, hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanil, sufentanil, alfentanil, remifentanil, levorphanol, dextromethorphan, phenazocine, pentazocine, cyclazocine, methadone, isomethadone, and propoxyphene. Suitable non-opioid analgesic agents include, but are not limited to, aspirin, celecoxib, rofecoxib, diclofenac, diflusinal, etodolac, fenoprofen, flurbiprofen, ibuprofen, ketoprofen, indomethacin, ketorolac, meclofenamate, mefanamic acid, nabumetone, naproxen, piroxicam, and sulindac.

In still another embodiment, the other therapeutic agent may be an anxiolytic agent. Suitable anxiolytic agents include, but are not limited to, buspirene, and benzodiazepines such as diazepam, lorazepam, oxazapam, chlorazepate, clonazepam, chlordiazepoxide, and alprazolam.

In addition to treating cancers and sensitizing a cancer cell to the cytotoxic effects of radiotherapy and chemotherapy, the present HDAC6Is are used in methods of treating diseases, conditions, and injuries to the central nervous system, such as neurological diseases, neurodegenerative disorders, and traumatic brain injuries (TBIs). In preferred embodiments, a present HDAC6I is capable of crossing the blood brain barrier to inhibit HDAC in the brain of the individual.

The present HDAC6I compounds also provide a therapeutic benefit in models of peripheral neuropathies, such as CMT. HDAC6 inhibitors have been found to cross the blood nerve barrier and rescue the phenotype observed in transgenic mice exhibiting symptons of distal hereditary motor neuropathy. Administration of HDAC6 inhibitors to symptomatic mice increased acetylated α-tubulin levels, restored proper mitochondrial motility and axonal transport, and increased muscle re-innervation. Other peripheral neuropathies include, but are not limited to, giant axonal neuropathy and various forms of mononeuropathies, polyneuropathies, autonomic neuropathies, and neuritis.

The present HDAC6I compounds also ameliorate associative memory loss following elevation of Aβ or of the tau protein. In this test, mice were infused with Aβ42 via cannulas implanted into dorsal hippocampus 15 minutes prior to training. The test compounds were dosed ip (25 mg/kg) 2 hours before training. Fear learning was assessed 24 hours later.

Contextual fear conditioning performed 24 hours after training shows a reduction of freezing in Aβ-infused mice compared to vehicle-infused mice. Treatment with a present compound ameliorates deficit in freezing responses in Aβ-infused mice and has no effect in vehicle-infused mice. A test compound alone does not affect the memory performance of the mice. In addition, treatment had no effects on motor, sensorial, or motivational skills assessed using the visible platform test in which the compounds are injected twice a day for two days. During these experiments, no signs of overt toxicity, including changes in food and liquid intake, weight loss, or changes in locomotion and exploratory behavior, are observed.

These results demonstrate that the HDAC6Is of the present invention are beneficial against impairment of associative memory following elevation of certain proteins including As and tau.

The present HDAC6Is therefore are useful for treating a neurological disease by administration of amounts of a present HDAC6I effective to treat the neurological disease or by administration of a pharmaceutical composition comprising amounts of a present HDAC6I effective to treat the neurological disease. The neurological diseases that can be treated include, but are not limited to, Huntington's disease, lupus, schizophrenia, multiple sclerosis, muscular dystrophy, dentatorubralpallidoluysian atrophy (DRRLA), spinal and bulbar muscular atrophy (SBMA), and fine spinocerebellar ataxias (SCA1, SCA2, SCA3/MJD (Machado-Joseph Disease), SCA6, and SCA7), drug-induced movement disorders, Creutzfeldt-Jakob disease, amyotrophic lateral sclerosis, Pick's disease, Alzheimer's disease, Lewy body dementia, corticobasal degeneration, dystonia, myoclonus, Tourette's syndrome, tremor, chorea, restless leg syndrome, Parkinson's disease, Parkinsonian syndromes, anxiety, depression, psychosis, manic depression, Friedreich's ataxia, Fragile X syndrome, spinal muscular dystrophy, Rett syndrome, Rubinstein-Taybi syndrome, Wilson's disease, multi-infarct state, CMT, GAN, and other peripheral neuropathies.

In an embodiment, the neurological disease treated is Huntington's disease, Parkinson's disease, Alzheimer's disease, Downs syndrome, spinal muscular atrophy, lupus, or schizophrenia.

Charcot-Marie-Tooth disease (CMT) is one of the most common inherited neurological disorders that affects about 1 in 2,500 people in the US. CMT affects both motor and sensory nerves which may result in foot drop and a high-stepped gait with frequent tripping or falls. Mutations in the small heat-shock protein 27 (HSPB1) cause axonal CMT or distal hereditary motor neuropathy (distal HMN). Expression of mutant HSPB1 decreased acetylated α-tubulin levels and induced severe axonal transport deficits. Pharmacological inhibition of histone deacetylase 6 (HDAC6)-induced α-tubulin deacetylation caused by the HDAC6I Tubastatin A corrects the axonal transport defects induced by HSPB1 mutations and rescues the CMT phenotype of symptomatic mutant HSPB1 mice. The pathogenic role of α-tubulin deacetylation has been demonstrated in mutant HSPB1-induced neuropathies and offers valuable perspectives for HDAC6 inhibitors as a therapeutic strategy for hereditary axonopathies. Compounds of the invention show potent HDAC6 isoform inhibition, high HDAC6 selectivity, and impressive α-tubulin acetylation in various cell lines.

Accordingly, in another embodiment, the neurological disease is Charcot-Marie-Tooth disease. Current studies using CMT2A mutant animals show that some of the HDAC6Is of the present invention are able to correct both motor and sensory problems in these animals, and to return their performance to levels similar to those shown by the wild type animals.

A present HDAC6I also can be used with a second therapeutic agent in methods of treating conditions, diseases, and injuries to the CNS. Such second therapeutic agents are those drugs known in the art to treat a particular condition, diseases, or injury, for example, but not limited to, lithium in the treatment of mood disorders, estradiol benzoate, and nicotinamide in the treatment of Huntington's disease.

The present HDAC6Is also are useful in the treatment of TBIs. Traumatic brain injury (TBI) is a serious and complex injury that occurs in approximately 1.4 million people each year in the United States. TBI is associated with a broad spectrum of symptoms and disabilities, including a risk factor for developing neurodegenerative disorders, such as Alzheimer's disease.

TBI produces a number of pathologies including axonal injury, cell death, contusions, and inflammation. The inflammatory cascade is characterized by proinflammatory cytokines and activation of microglia which can exacerbate other pathologies. Although the role of inflammation in TBI is well established, no efficacious anti-inflammatory therapies are currently available for the treatment of TBI.

Several known HDAC inhibitors have been found to be protective in different cellular and animal models of acute and chronic neurodegenerative injury and disease, for example, Alzheimer's disease, ischemic stroke, multiple sclerosis (MS), Huntington's disease (HD), amyotrophic lateral sclerosis (ALS), spinal muscular atrophy (SMA), and spinal and bulbar muscular atrophy (SBMA). A recent study in experimental pediatric TBI reported a decrease in hippocampal CA3 histone H3 acetylation lasting hours to days after injury. These changes were attributed to documented upstream excitotoxic and stress cascades associated with TBI. HDAC6Is also have been reported to have anti-inflammatory actions acting through acetylation of non-histone proteins. The HDAC6 selective inhibitor, 4-dimethylamino-N-[5-(2-mercaptoacetylamino)pentyl]benzamide (DMA-PB), was found to be able to increase histone H3 acetylation and reduce microglia inflammatory response following traumatic brain injury in rats, which demonstrates the utility of HDAC6Is as therapeutics for inhibiting neuroinflammation associated with TBI.

The present HDAC6Is therefore also are useful in the treatment of inflammation and strokes, and in the treatment of autism and autism spectrum disorders. The present HDAC6Is further can be used to treat parasitic infections, (e.g., malaria, toxoplasmosis, trypanosomiasis, helminthiasis, and protozoal infections (see Andrews et al. Int. J. Parasitol. 2000, 30(6), 761-768).

The present HDAC6Is also can be used as imaging agents. In particular, by providing a radiolabeled, isotopically labeled, or fluorescently-labeled HDAC6I, the labeled compound can image HDACs, tissues expressing HDACs, and tumors. Labeled HDAC6Is of the present invention also can image patients suffering from a cancer, or other HDAC-mediated diseases, e.g., stroke, by administration of an effective amount of the labeled compound or a composition containing the labeled compound. In preferred embodiments, the labeled HDAC6I is capable of emitting positron radiation and is suitable for use in positron emission tomography (PET). Typically, a labeled HDAC6I of the present invention is used to identify areas of tissues or targets that express high concentrations of HDACs. The extent of accumulation of labeled HDAC6I can be quantified using known methods for quantifying radioactive emissions. In addition, the labeled HDAC6I can contain a fluorophore or similar reporter capable of tracking the movement of particular HDAC isoforms or organelles in vitro.

The present HDAC6Is useful in the imaging methods contain one or more radioisotopes capable of emitting one or more forms of radiation suitable for detection by any standard radiology equipment, such as PET, SPECT, gamma cameras, MRI, and similar apparatus. Preferred isotopes including tritium (³H) and carbon (¹¹C). Substituted HDAC6Is of the present invention also can contain isotopes of fluorine (¹⁸F) and iodine (¹²³I) for imaging methods. Typically, a labeled HDAC6I of the present invention contains an alkyl or aryl group having a ¹¹C label, i.e., a ¹¹C-methyl group, or an alkyl or aryl group substituted with ¹⁸F, ¹²³I, ¹²⁵I, ¹³¹I, or a combination thereof.

Fluorescently-labeled HDAC6Is of the present invention also can be used in the imaging method of the present invention. Such compounds have an FITC, carbocyanine moiety, or other fluorophore which will allow visualization of the HDAC proteins in vitro.

The labeled HDAC6Is and methods of use can be in vivo, and particularly on humans, and for in vitro applications, such as diagnostic and research applications, using body fluids and cell samples. Imaging methods using a labeled HDAC6I of the present invention are discussed in WO 03/060523, designating the U.S. and incorporated in its entirety herein. Typically, the method comprises contacting cells or tissues with a radiolabeled, isotopically labeled, fluorescently labeled, or tagged (such as biotin tagged) compound of the invention, and making a radiographic, fluorescent, or similar type of image depending on the visualization method employed, i.e., in regard to radiographic images, in a sufficient amount to provide about 1 to about 30 mCi of the radiolabeled compound.

Preferred imaging methods include the use of labeled HDAC6Is of the present invention which are capable of generating at least a 2:1 target to background ratio of radiation intensity, or more preferably about a 5:1, about 10:1, or about 15:1 ratio of radiation intensity between target and background.

In preferred methods, the labeled HDAC6Is of the present invention are excreted from tissues of the body quickly to prevent prolonged exposure to the radiation of the radiolabeled compound administered to the individual. Typically, labeled HDAC6Is of the present invention are eliminated from the body in less than about 24 hours. More preferably, labeled HDAC6Is are eliminated from the body in less than about 16 hours, 12 hours, 8 hours, 6 hours, 4 hours, 2 hours, 90 minutes, or 60 minutes. Typically, preferred labeled HDAC6Is are eliminated in about 60 to about 120 minutes.

In addition to isotopically labeled and fluorescently labeled derivatives, the present invention also embodies the use of derivatives containing tags (such as biotin) for the identification of biomolecules associated with the HDAC isoforms of interest for diagnostic, therapeutic, or research purposes.

The present HDAC6Is also are useful in the treatment of autoimmune diseases and inflammations. Compounds of the present invention are particularly useful in overcoming graft and transplant rejections and in treating forms of arthritis.

Despite successes of modern transplant programs, the nephrotoxicity, cardiovascular disease, diabetes, and hyperlipidemia associated with current therapeutic regimens, plus the incidence of post-transplant malignancies and graft loss from chronic rejection, drive efforts to achieve long-term allograft function in association with minimal immunosuppression. Likewise, the incidence of inflammatory bowel disease (IBD), including Crohn's disease and ulcerative colitis, is increasing. Animal studies have shown that T regulatory cells (Tregs) expressing the forkhead transcription family member, Foxp3, are key to limiting autoreactive and alloreactive immunity. Moreover, after their induction by costimulation blockade, immunosuppression, or other strategies, Tregs may be adoptively transferred to naïve hosts to achieve beneficial therapeutic effects. However, attempts to develop sufficient Tregs that maintain their suppressive functions post-transfer in clinical trials have failed. Murine studies show that HDAC6Is limit immune responses, at least in significant part, by increasing Treg suppressive functions (R. Tao et al., Nat. Med, 13, 1299-1307 (2007)), and that selective targeting of HDAC6 is especially efficacious in this regard.

With organ transplantation, rejection begins to develop in the days immediately post-transplant, such that prevention rather than treatment of rejection is a paramount consideration. The reverse applies in autoimmunity, wherein a patient presents with the disease already causing problems. Accordingly, HDAC6−/− mice treated for 14 days with low-dose RPM (rapamycin) are assessed for displaying signs of tolerance induction and resistance to the development of chronic rejection, a continuing major loss of graft function long-term in the clinical transplant population. Tolerance is assessed by testing whether mice with long-surviving allografts reject a subsequent third-party cardiac graft and accept additional donor allografts without any immunosuppression, as can occur using a non-selective HDAC6I plus RPM. These in vivo studies are accompanied by assessment of ELISPOT and MLR activities using recipient lymphocytes challenged with donor cells. Protection against chronic rejection is assessed by analysis of host anti-donor humoral responses and analysis of graft transplant arteriosclerosis and interstitial fibrosis in long-surviving allograft recipients.

The importance of HDAC6 targeting is assessed in additional transplant models seeking readouts of biochemical significance, as is monitored clinically. Thus, the effects of HDAC6 in targeting in renal transplant recipients (monitoring BUN, proteinuria) and islet allografts (monitoring blood glucose levels) are assessed. Renal transplants are the most common organ transplants performed, and the kidney performs multiple functions, e.g., regulating acid/base metabolism, blood pressure, red cell production, such that efficacy in this model indicates the utility of HDAC6 targeting. Likewise, islet transplantation is a major unmet need given that clinical islet allografts are typically lost after the first one or two years post-transplant. Having a safe and non-toxic means to extend islet survival without maintenance CNI therapy would be an important advance. Transplant studies also are strengthened by use of mice with floxed HDAC6. Using existing Foxp3-Cre mice, for example, the effects of deletion of HDAC6 just in Tregs is tested. This approach can be extended to targeting of HDAC6 in T cells (CD4-Cre) and dendritic cells (CD11c-Cre), for example. Using tamoxifen-regulated Cre, the importance of HDAC6 in induction vs. maintenance of transplants (with implications for short-term vs. maintenance HDAC6I therapy) is assessed by administering tamoxifen and inducing HDAC6 deletion at varying periods post-transplant.

Studies of autoimmunity also are undertaken. In this case, interruption of existing disease is especially important, and HDAC6 targeting can be efficacious without any requirement for additional therapy (in contrast to a need for brief low-dose RPM in the very aggressive, fully MHC-mismatched transplant models). Studies in mice with colitis indicated that HDAC6−/− Tregs were more effective than WT Tregs in regulating disease, and tubacin was able to rescue mice if treatment was begun once colitis had developed. These studies are extended by assessing whether deletion of HDAC6 in Tregs (Foxp3/Cre) vs. T cells (CD4=Cre) vs. DC (CD11 c-Cre) differentially affect the development and severity of colitis. Similarly, control of colitis is assessed by inducing HDAC6 deletion at varying intervals after the onset of colitis with tamoxifen-regulated Cre.

The present compounds are envisioned to demonstrate anti-arthritic efficacy in a collagen-induced arthritis model in DBA1/J mice. In this test, DBA1/J mice (male, 7-8 weeks) are used, with 8 animals per group. Systemic arthritis is induced with bovine collagen type II and CFA, plus an IFA booster injection on day 21. A present HDAC6I is dosed at 50 mg/kg and 100 mg/kg on day 28 for 2 consecutive weeks, and the effects determined from the Average Arthritic Score vs. Days of Treatment data.

Despite efforts to avoid graft rejection through host-donor tissue type matching, in the majority of transplantation procedures, immunosuppressive therapy is critical to the viability of the donor organ in the host. A variety of immunosuppressive agents have been employed in transplantation procedures, including azathioprine, methotrexate, cyclophosphamide, FK-506, rapamycin, and corticosteroids.

The present HDAC6Is are potent immunosuppressive agents that suppress humoral immunity and cell-mediated immune reactions, such as allograft rejection, delayed hypersensitivity, experimental allergic encephalomyelitis, Freund's adjuvant arthritis and graft versus host disease. HDAC6Is of the present invention are useful for the prophylaxis of organ rejection subsequent to organ transplantation, for treatment of rheumatoid arthritis, for the treatment of psoriasis, and for the treatment of other autoimmune diseases, such as type I diabetes, Crohn's disease, and lupus.

A therapeutically effective amount of a present HDAC6I can be used for immunosuppression including, for example, to prevent organ rejection or graft vs. host disease, and to treat diseases and conditions, in particular, autoimmune and inflammatory diseases and conditions. Examples of autoimmune and inflammatory diseases include, but are not limited to, Hashimoto's thyroiditis, pernicious anemia, Addison's disease, psoriasis, diabetes, rheumatoid arthritis, systemic lupus erythematosus, dermatomyositis, Sjogren's syndrome, multiple sclerosis, myasthenia gravis, Reiter's syndrome, arthritis (rheumatoid arthritis, arthritis chronic progrediente, and arthritis deformans) and rheumatic diseases, autoimmune hematological disorder (hemolytic anaemia, aplastic anaemia, pure red cell anaemia and idiopathic thrombocytopaenia), polychondritis, scleroderma, Wegener granulomatosis, chronic active hepatitis, Steven-Johnson syndrome, idiopathic sprue, autoimmune inflammatory bowel disease (ulcerative colitis and Crohn's disease), endocrine opthalmopathy, Graves disease, sarcoidosis, primary biliary cirrhosis, juvenile diabetes (diabetes mellitus type I), uveitis (anterior and posterior), keratoconjunctivitis sicca and vernal keratoconjunctivitis, interstitial lung fibrosis, psoriatic arthritis, and glomerulonephritis.

A present HDAC6I can be used alone, or in conjunction with a second therapeutic agent known to be useful in the treatment of autoimmune diseases, inflammations, transplants, and grafts, such as cyclosporin, rapamycin, methotrexate, cyclophosphamide, azathioprine, corticosteroids, and similar agents known to persons skilled in the art.

Additional diseases and conditions mediated by HDACs, and particularly HDAC6, include, but are not limited to asthma, cardiac hypertrophy, giant axonal neuropathy, mononeuropathy, mononeuritis, polyneuropathy, autonomic neuropathy, neuritis in general, and neuropathy in general. These disease and conditions also can be treated by a method of the present invention.

In the present method, a therapeutically effective amount of one or more HDAC6I of the present invention, typically formulated in accordance with pharmaceutical practice, is administered to a human being in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

A present HDAC6I can be administered by any suitable route, for example by oral, buccal, inhalation, topical, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique.

Pharmaceutical compositions include those wherein a present HDAC6I is present in a sufficient amount to be administered in an effective amount to achieve its intended purpose. The exact formulation, route of administration, and dosage is determined by an individual physician in view of the diagnosed condition or disease. Dosage amount and interval can be adjusted individually to provide levels of a present HDAC6I that are sufficient to maintain therapeutic effects.

Toxicity and therapeutic efficacy of the present HDAC6I compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, which is expressed as the ratio between LD₅₀ and ED₅₀. Compounds that exhibit high therapeutic indices are preferred. The data obtained from such procedures can be used in formulating a dosage range for use in humans. The dosage preferably lies within a range of circulating compound concentrations that include the ED₅₀ with little or no toxicity. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of a present HDAC6I required for use in therapy varies with the nature of the condition being treated, the length of time that activity is desired, and the age and the condition of the patient, and ultimately is determined by the attendant physician. Dosage amounts and intervals can be adjusted individually to provide plasma levels of the HDAC6I that are sufficient to maintain the desired therapeutic effects. The desired dose conveniently can be administered in a single dose, or as multiple doses administered at appropriate intervals, for example as one, two, three, four, or more subdoses per day. Multiple doses often are desired, or required. For example, a present HDAC6I can be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

The dosage of a composition containing a present HDAC6I, or a composition containing the same, can be from about 1 ng/kg to about 200 mg/kg, about 1 μg/kg to about 100 mg/kg, or about 1 mg/kg to about 50 mg/kg of body weight. The above dosages are exemplary of the average case, but there can be individual instances in which higher or lower dosages are merited, and such are within the scope of this invention. In practice, the physician determines the actual dosing regimen that is most suitable for an individual patient, which can vary with the age, weight, and response of the particular patient.

A present HDAC6I used in a method of the present invention typically is administered in an amount of about 0.005 to about 500 milligrams per dose, about 0.05 to about 250 milligrams per dose, or about 0.5 to about 100 milligrams per dose. For example, a present HDAC6I can be administered, per dose, in an amount of about 0.005, 0.05, 0.5, 5, 10, 20, 30, 40, 50, 100, 150, 200, 250, 300, 350, 400, 450, or 500 milligrams, including all doses between 0.005 and 500 milligrams.

The HDAC6Is of the present invention typically are administered in admixture with a pharmaceutical carrier selected with regard to the intended route of administration and standard pharmaceutical practice. Pharmaceutical compositions for use in accordance with the present invention are formulated in a conventional manner using one or more physiologically acceptable carriers comprising excipients and auxiliaries that facilitate processing of the present HDAC6Is.

The term “carrier” refers to a diluent, adjuvant, or excipient, with which a present HDAC6I is administered. Such pharmaceutical carriers can be liquids, such as water and oils, including those of petroleum, animal, vegetable or synthetic origin, such as peanut oil, soybean oil, mineral oil, sesame oil, and the like. The carriers can be saline, gum acacia, gelatin, starch paste, talc, keratin, colloidal silica, urea, and the like. In addition, auxiliary, stabilizing, thickening, lubricating, and coloring agents can be used. The pharmaceutically acceptable carriers are sterile. Water is a preferred carrier when a present HDAC6I is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The present compositions, if desired, can also contain minor amounts of wetting or emulsifying agents, or pH buffering agents.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of a present HDAC6I is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a present HDAC6I. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a present compound.

When a therapeutically effective amount of a present HDAC6I is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains an isotonic vehicle. A present HDAC6I can be infused with other fluids over a 10-30 minute span or over several hours.

The present HDAC6Is can be readily combined with pharmaceutically acceptable carriers well-known in the art. Such carriers enable the active agents to be formulated as tablets, pills, dragees, capsules, liquids, gels, syrups, slurries, suspensions and the like, for oral ingestion by a patient to be treated. Pharmaceutical preparations for oral use can be obtained by adding a present HDAC6I to a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

A present HDAC6I can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a present HDAC6I can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

A present HDAC6I also can be formulated in rectal compositions, such as suppositories or retention enemas, e.g., containing conventional suppository bases. In addition to the formulations described previously, a present HDAC6I also can be formulated as a depot preparation. Such long-acting formulations can be administered by implantation (for example, subcutaneously or intramuscularly) or by intramuscular injection. Thus, for example, a present HDAC6I can be formulated with suitable polymeric or hydrophobic materials (for example, as an emulsion in an acceptable oil) or ion exchange resins.

In particular, a present HDAC6I can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. The present HDAC6Is also can be injected parenterally, for example, intravenously, intramuscularly, subcutaneously, or intracoronarily. For parenteral administration, the present HDAC6Is are best used in the form of a sterile aqueous solution which can contain other substances, for example, salts or monosaccharides, such as mannitol or glucose, to make the solution isotonic with blood.

As an additional embodiment, the present invention includes kits which comprise one or more compounds or compositions packaged in a manner that facilitates their use to practice methods of the invention. In one simple embodiment, the kit includes a compound or composition described herein as useful for practice of a method (e.g., a composition comprising a present HDAC6I and an optional second therapeutic agent), packaged in a container, such as a sealed bottle or vessel, with a label affixed to the container or included in the kit that describes use of the compound or composition to practice the method of the invention. Preferably, the compound or composition is packaged in a unit dosage form. The kit further can include a device suitable for administering the composition according to the intended route of administration, for example, a syringe, drip bag, or patch. In another embodiment, the present compounds is a lyophilate. In this instance, the kit can further comprise an additional container which contains a solution useful for the reconstruction of the lyophilate.

Prior HDAC6Is possessed properties that hindered their development as therapeutic agents. In accordance with an important feature of the present invention, the present HDAC6Is were synthesized and evaluated as inhibitors for HDAC. The present compounds demonstrate an increased HDAC6 potency and selectivity against HDAC1 and HDAC8. The improved properties of the present compounds indicate that the present compounds are useful for applications such as, but not limited to, immunosuppresssive and neuroprotective agents. For example, compounds of the present invention typically have an inhibitor potency (IC₅₀) to HDAC6 of less than 100 μM, less than 25 μM, less than 10 μM, less than 1 μM, less than 0.5 μM, and less than 0.2 μM.

### Use of the HDAC6 Inhibitors.

An HDAC6I of the present invention can be used alone, or in conjunction with a second therapeutic agent known to be useful in the treatment of various diseases including autoimmune diseases, inflammations, or rejection of transplants and grafts, such as cyclosporin, rapamycin, methotrexate, cyclophosphamide, azathioprine, corticosteroids, and similar agents known to persons skilled in the art.

Additional diseases and conditions mediated by HDACs, and particularly HDAC6, include, but are not limited to asthma, cardiac hypertrophy, giant axonal neuropathy, mononeuropathy, mononeuritis, polyneuropathy, autonomic neuropathy, neuritis in general, and neuropathy in general. These diseases and conditions also can be treated by a method of the present invention.

In the present method, a therapeutically effective amount of one or more HDAC6I of the present invention, typically formulated in accordance with pharmaceutical practice, is administered to a human being in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

A present HDAC6I can be administered by any suitable route, for example by oral, buccal, inhalation, topical, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique.

Pharmaceutical compositions include those wherein a present HDAC6I is present in a sufficient amount to be administered in an effective amount to achieve its intended purpose. The exact formulation, route of administration, and dosage is determined by an individual physician in view of the diagnosed condition or disease. Dosage amount and interval can be adjusted individually to provide levels of a present HDAC6I that is sufficient to maintain therapeutic effects.

Toxicity and therapeutic efficacy of the present HDAC6I compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, which is expressed as the ratio between LD₅₀ and ED₅₀. Compounds that exhibit high therapeutic indices are preferred. The data obtained from such procedures can be used in formulating a dosage range for use in humans. The dosage preferably lies within a range of circulating compound concentrations that include the ED₅₀ with little or no toxicity. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of a present HDAC6I required for use in therapy varies with the nature of the condition being treated, the length of time that activity is desired, and the age and the condition of the patient, and ultimately is determined by the attendant physician. Dosage amounts and intervals can be adjusted individually to provide plasma levels of the HDAC6I that are sufficient to maintain the desired therapeutic effects. The desired dose conveniently can be administered in a single dose, or as multiple doses administered at appropriate intervals, for example as one, two, three, four, or more subdoses per day. Multiple doses often are desired, or required. For example, a present HDAC6I can be administered at a frequency of; four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

The dosage of a composition containing a present HDAC6I, or a composition containing the same, can be from about 1 ng/kg to about 200 mg/kg, about 1 μg/kg to about 100 mg/kg, or about 1 mg/kg to about 50 mg/kg of body weight. The dosage of a composition may be at any dosage including, but not limited to, about 1 μg/kg, 10 μg/kg to 200 mg/kg. The above dosages are exemplary of the average case, but there can be individual instances in which higher or lower dosages are merited, and such are within the scope of this invention. In practice, the physician determines the actual dosing regimen that is most suitable for an individual patient, which can vary with the age, weight, and response of the particular patient.

A present HDAC6I used in a method of the present invention typically is administered in an amount of about 0.005 to about 500 milligrams per dose, about 0.05 to about 250 milligrams per dose, or about 0.5 to about 100 milligrams per dose. For example, a present HDAC6I can be administered, per dose, in an amount of about 0.005, 0.05, 0.5, 5, 10, 20, 30, 40, 50, 100, 150, 200, 250, 300, 350, 400, 450, or 500 milligrams, including all doses between 0.005 and 500 milligrams.

The HDAC6Is of the present invention typically are administered in admixture with a pharmaceutical carrier selected with regard to the intended route of administration and standard pharmaceutical practice. Pharmaceutical compositions for use in accordance with the present invention are formulated in a conventional manner using one or more physiologically acceptable carriers comprising excipients and auxiliaries that facilitate processing of the present HDAC6Is.

The term “carrier” refers to a diluent, adjuvant, or excipient, with which a present HDAC6I is administered. Such pharmaceutical carriers can be liquids, such as water and oils, including those of petroleum, animal, vegetable or synthetic origin, such as peanut oil, soybean oil, mineral oil, sesame oil, and the like. The carriers can be saline, gum acacia, gelatin, starch paste, talc, keratin, colloidal silica, urea, and the like. In addition, auxiliary, stabilizing, thickening, lubricating, and coloring agents can be used. The pharmaceutically acceptable carriers are sterile. Water is a preferred carrier when a present HDAC6I is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The present compositions, if desired, can also contain minor amounts of wetting or emulsifying agents, or pH buffering agents.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of a present HDAC6I is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a present HDAC6I. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a present compound.

When a therapeutically effective amount of a present HDAC6I is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains an isotonic vehicle. A present HDAC6I can be infused with other fluids over a 10-30 minute span or over several hours.

The present HDAC6Is can be readily combined with pharmaceutically acceptable carriers well-known in the art. Such carriers enable the active agents to be formulated as tablets, pills, dragees, capsules, liquids, gels, syrups, slurries, suspensions and the like, for oral ingestion by a patient to be treated. Pharmaceutical preparations for oral use can be obtained by adding a present HDAC6I to a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

A present HDAC6I can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a present HDAC6I can be prepared as appropriate oily injection suspensions. Suitable lipophilic solvents or vehicles include fatty oils or synthetic fatty acid esters. Aqueous injection suspensions can contain substances which increase the viscosity of the suspension.

Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

A present HDAC6I also can be formulated in rectal compositions, such as suppositories or retention enemas, e.g., containing conventional suppository bases. In addition to the formulations described previously, a present HDAC6I also can be formulated as a depot preparation. Such long-acting formulations can be administered by implantation (for example, subcutaneously or intramuscularly) or by intramuscular injection. Thus, for example, a present HDAC6I can be formulated with suitable polymeric or hydrophobic materials (for example, as an emulsion in an acceptable oil) or ion exchange resins.

In particular, a present HDAC6I can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. The present HDAC6Is also can be injected parenterally, for example, intravenously, intramuscularly, subcutaneously, or intracoronarily. For parenteral administration, the present HDAC6Is are best used in the form of a sterile aqueous solution which can contain other substances, for example, salts or monosaccharides, such as mannitol or glucose, to make the solution isotonic with blood.

As an additional embodiment, the present invention includes kits which comprise one or more compounds or compositions packaged in a manner that facilitates their use to practice methods of the invention. In one simple embodiment, the kit includes a compound or composition described herein as useful for practice of a method (e.g., a composition comprising a present HDAC6I and an optional second therapeutic agent), packaged in a container, such as a sealed bottle or vessel, with a label affixed to the container or included in the kit that describes use of the compound or composition to practice the method of the invention. Preferably, the compound or composition is packaged in a unit dosage form. The kit further can include a device suitable for administering the composition according to the intended route of administration, for example, a syringe, drip bag, or patch. In another embodiment, the selected compound is a lyophilate. In this instance, the kit can further comprise an additional container which contains a solution useful for the reconstruction of the lyophilate.

A number of the prior HDAC6Is possess properties that are likely to hinder their development as therapeutic agents for diseases other than cancer due to the fact that they often show activity against a number of the known HDACs. Accordingly, an important feature of the present invention relates to the fact that compounds of the present invention show isoform selectivity. The present compounds demonstrate an increased inhibitory potency and selectivity for HDAC6 relative to other HDACs, and in particular greater selectivity for Class II over Class I. The improved properties of the present compounds indicate that these compounds should be useful for applications such as, but not limited to immunosuppresssive and neuroprotective agents, as well as Alzheimer's disease, depression, Rett syndrome, Charcot Marie Tooth disease, brain cancer, and others. For example, compounds of the present invention typically have a binding affinity (IC₅₀) to HDAC6 of less than 1 μM, and in some cases less than 10 nM.

### Definitions

The following terms and expressions used herein have the indicated meanings.

Terms used herein may be preceded and/or followed by a single dash, “-”, or a double dash, “=”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” unless a dash indicates otherwise. For example, C₁-C₆ alkoxycarbonyloxy and —OC(O)OC₁-C₆ alkyl indicate the same functionality; similarly, arylalkyl and -alkylaryl indicate the same functionality.

“Acetyl” means a group of formula —C(O)CH₃.

“Alkenyl” means a straight or branched chain hydrocarbon containing from 2 to 10 carbons, unless otherwise specified, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

“Alkynyl” means a straight or branched chain hydrocarbon group containing from 2 to 10 carbon atoms and containing at least one carbon-carbon triple bond. Representative examples of alkynyl include, but are not limited, to ethynyl, 1-propynyl, 2-propynyl, 1-butynyl, 3-butynyl, and 2-pentynyl.

“Alkoxy” means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

“Alkyl” means a straight or branched chain hydrocarbon containing from 1 to 10 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂—, —CH₂CH₂C(CH₃)₂—, and —CH₂CH(CH₂CH₃)CH₂—.

“Aryl” means a phenyl (i.e., monocyclic aryl), or a bicyclic ring system containing at least one phenyl ring or an aromatic bicyclic ring containing only carbon atoms in the aromatic bicyclic ring system. The bicyclic aryl can be azulenyl, naphthyl, and the like. The aryl is attached to the parent molecular moiety through any carbon atom contained within the aryl ring system. One or two hydrogen atoms that are part of the aryl group may be replaced with substituents selected from the group of F, Cl, CH₃, CHF₂, CF₃, OCH, OCHF₂, OCF₃, SCF₃, and CN; or two adjacent hydrogen atoms may be replaced with the moiety —OCH₂O—. In certain embodiments, the aryl group is phenyl or naphthyl. In certain other embodiments, the aryl group is phenyl.

“Cyano” and “nitrile” mean a —CN group.

“Cycloalkyl” means a 3- to 6-membered monocyclic ring. The cycloalkyl may be saturated or unsaturated, but not aromatic. Representative cycloalkyl include, but are not limited to cyclopropyl, cyclobutyl, cyclopentyl, cyclopentenyl, cyclohexyl, and cyclohexenyl.

“Halo” or “halogen” means —Cl, —Br, —I, or —F.

“Haloalkyl” means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

“Heteroaryl” means a monocyclic heteroaryl or a bicyclic ring system containing at least one heteroaromatic ring. The monocyclic heteroaryl can be a 5- or 6-membered ring. The 5-membered ring consists of two double bonds and one, two, three, or four nitrogen atoms and optionally one oxygen or sulfur atom, or if said oxygen or sulfur atom are present, then nitrogen atoms may be absent. The 6-membered ring consists of three double bonds and one, two, three, or four nitrogen atoms. The 5- or 6-membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. The fused cycloalkyl or heterocyclyl portion of the bicyclic heteroaryl group is optionally substituted with one or two groups which are independently oxo or thioxo. When the bicyclic heteroaryl contains a fused cycloalkyl, cycloalkenyl, or heterocyclyl ring, then the bicyclic heteroaryl group is connected to the parent molecular moiety through any carbon or nitrogen atom contained within the monocyclic heteroaryl portion of the bicyclic ring system. When the bicyclic heteroaryl is a monocyclic heteroaryl fused to a phenyl ring, then the bicyclic heteroaryl group is connected to the parent molecular moiety through any carbon atom or nitrogen atom within the bicyclic ring system. Representative examples of bicyclic heteroaryl include, but are not limited to, benzimidazolyl, benzofuranyl, benzothienyl, benzoxadiazolyl, benzothiadiazolyl, benzothiazolyl, cinnolinyl, 5,6-dihydroquinolin-2-yl, 5,6-dihydroisoquinolin-1-yl, furopyridinyl, indazolyl, indolyl, isoquinolinyl, naphthyridinyl, quinolinyl, purinyl, 5,6,7,8-tetrahydroquinolin-2-yl, 5,6,7,8-tetrahydroquinolin-3-yl, 5,6,7,8-tetrahydroquinolin-4-yl, 5,6,7,8-tetrahydroisoquinolin-1-yl, thienopyridinyl, and 4,5,6,7-tetrahydrobenzo[c][1,2,5]oxadiazolyl. In certain embodiments, the fused bicyclic heteroaryl is a 5- or 6-membered monocyclic heteroaryl ring fused to either a phenyl ring, a 5- or 6-membered monocyclic cycloalkyl, a 5- or 6-membered monocyclic cycloalkenyl, a 5- or 6-membered monocyclic heterocyclyl, or a 5- or 6-membered monocyclic heteroaryl, wherein the fused cycloalkyl, cycloalkenyl, and heterocyclyl groups are optionally substituted with one or two groups which are independently oxo or thioxo. In certain embodiments of the disclosure, the heteroaryl group is furyl, imidazolyl, pyridinyl, pyrimidinyl, pyrazinyl, pyrazolyl, pyrrolyl, thiazolyl, thienyl, triazolyl, benzimidazolyl, benzofuranyl, indazolyl, indolyl, quinolinyl, and the like. One or two hydrogen atoms that are part of the heteroaryl group may be replaced with substituents selected from the group of F, Cl, CH₃, CHF₂, CF₃, OCH₃, OCHF₂, OCF₃, SCF₃, and CN.

“Heterocyclyl” means a monocyclic 4- to 7-membered heterocyclic ring containing one or more heteroatoms independently selected from O, N, and S where the ring is saturated or unsaturated, but not aromatic. Representative examples of monocyclic heterocycle include, but are not limited to, azetidinyl, hexahydroazepinyl, imidazolinyl, imidazolidinyl, isothiazolinyl, isothiazolidinyl, morpholinyl, oxadiazolinyl, oxadiazolidinyl, oxazolinyl, oxazolidinyl, oxetanyl, piperazinyl, piperidinyl, pyrazolinyl, pyrazolidinyl, pyrrolinyl, pyrrolidinyl, thiopyranyl. In certain embodiments, the heterocyclyl is imidazolinyl, pyrrolidinyl, piperidinyl, or piperazinyl.

“Deuterated” means containing one or more carbon-deuterium bonds that replace carbon-hydrogen bonds, up to the maximum of all carbon-hydrogen bonds contained in the group under consideration.

The present invention is directed to novel HDAC6Is of formulas I and Ib-Iqq and their use in therapeutic treatments of, for example, cancers, inflammations, traumatic brain injuries, neurodegenerative disorders, neurological diseases, peripheral neuropathies, strokes, hypertension, autoimmune diseases, inflammatory diseases, and malaria. The present HDAC6Is also increase the sensitivity of a cancer cell to the cytotoxic effects of radiotherapy and/or chemotherapy. In some embodiments, the present HDAC6Is selectively inhibit HDAC6 over other HDAC isozymes.

The present invention is described in connection with preferred embodiments. However, it should be appreciated that the invention is not limited to the disclosed embodiments. It is understood that, given the description of the embodiments of the invention herein, various modifications can be made by a person skilled in the art. Such modifications are encompassed by the claims below.

The term “a disease or condition wherein inhibition of HDAC provides a benefit” pertains to a condition in which HDAC and/or the action of HDAC is important or necessary, e.g., for the onset, progress, expression of that disease or condition, or a disease or a condition which is known to be treated by an HDAC inhibitor (such as, e.g., TSA, pivaloyloxymethylbutane (AN-9; Pivanex), FK-228 (Depsipeptide), PXD-101, NVP-LAQ824, SAHA, MS-275, and/or MGCD0103). Examples of such conditions include, but are not limited to, cancer, psoriasis, fibroproliferative disorders (e.g., liver fibrosis), smooth muscle proliferative disorders (e.g., atherosclerosis, restenosis), neurodegenerative diseases (e.g., Alzheimer's, Parkinson's, Huntington's chorea, amyotropic lateral sclerosis, spino-cerebellar degeneration, Rett syndrome), peripheral neuropathies (Charcot-Marie-Tooth disease, Giant Axonal Neuropathy (GAN)), inflammatory diseases (e.g., osteoarthritis, rheumatoid arthritis, colitis), diseases involving angiogenesis (e.g., cancer, rheumatoid arthritis, psoriasis, diabetic retinopathy), hematopoietic disorders (e.g., anemia, sickle cell anemia, thalassemia), fungal infections, parasitic infections (e.g., malaria, trypanosomiasis, helminthiasis, protozoal infections), bacterial infections, viral infections, and conditions treatable by immune modulation (e.g., multiple sclerosis, autoimmune diabetes, lupus, atopic dermatitis, allergies, asthma, allergic rhinitis, inflammatory bowel disease; and for improving grafting of transplants). One of ordinary skill in the art is readily able to determine whether a compound treats a disease or condition mediated by HDAC for any particular cell type, for example, by assays which conveniently can be used to assess the activity of particular compounds.

The term “second therapeutic agent” refers to a therapeutic agent different from a present HDAC6I and that is known to treat the disease or condition of interest. For example, when a cancer is the disease or condition of interest, the second therapeutic agent can be a known chemotherapeutic drug, like taxol, or radiation, for example.

The term “HDAC” refers to a family of enzymes that remove acetyl groups from a protein, for example, the ε-amino groups of lysine residues at the N-terminus of a histone. The HDAC can be a human HDAC, including, HDACI, HDAC2, HDAC3, HDAC4, HDAC5, HDAC6, HDAC7, HDAC8, HDAC9, HDAC10, and HDAC11. The HDAC also can be derived from a protozoal or fungal source.

The terms “treat,” “treating,” “treatment,” and the like refer to eliminating, reducing, relieving, reversing, and/or ameliorating a disease or condition and/or symptoms associated therewith. Although not precluded, treating a disease or condition does not require that the disease, condition, or symptoms associated therewith be completely eliminated, including the treatment of acute or chronic signs, symptoms, and/or malfunctions. As used herein, the terms “treat,” “treating,” “treatment,” and the like may include “prophylactic treatment,” which refers to reducing the probability of developing or redeveloping a disease or condition, or of a recurrence of a previously-controlled disease or condition, in a subject who does not have, but is at risk of or is susceptible to, developing or redeveloping a disease or condition or a recurrence of the disease or condition. “Treatment” therefore also includes relapse prophylaxis or phase prophylaxis. The term “treat” and synonyms contemplate administering a therapeutically effective amount of a compound of the invention to an individual in need of such treatment. A treatment can be orientated symptomatically, for example, to suppress symptoms. It can be effected over a short period, be oriented over a medium term, or can be a long-term treatment, for example within the context of a maintenance therapy.

The term “therapeutically effective amount” or “effective dose” as used herein refers to an amount of the active ingredient(s) that, when administered, is (are) sufficient, to efficaciously deliver the active ingredient(s) for the treatment of condition or disease of interest to an individual in need thereof. In the case of a cancer or other proliferation disorder, the therapeutically effective amount of the agent may reduce (i.e., retard to some extent and preferably stop) unwanted cellular proliferation; reduce the number of cancer cells; reduce the tumor size; inhibit (i.e., retard to some extent and preferably stop) cancer cell infiltration into peripheral organs; inhibit (i.e., retard to some extent and preferably stop) tumor metastasis; inhibit, to some extent, tumor growth; reduce HDAC signaling in the target cells; and/or relieve, to some extent, one or more of the symptoms associated with the cancer. To the extent the administered compound or composition prevents growth and/or kills existing cancer cells, it may be cytostatic and/or cytotoxic.

“Concurrent administration,” “administered in combination,” “simultaneous administration,” and similar phrases mean that two or more agents are administered concurrently to the subject being treated. By “concurrently,” it is meant that each agent is administered either simultaneously or sequentially in any order at different points in time. However, if not administered simultaneously, it is meant that they are administered to an individual in a sequence and sufficiently close in time so as to provide the desired therapeutic effect and can act in concert. For example, a present HDAC6I can be administered at the same time or sequentially in any order at different points in time as a second therapeutic agent. A present HDAC6I and the second therapeutic agent can be administered separately, in any appropriate form and by any suitable route. When a present HDAC6I and the second therapeutic agent are not administered concurrently, it is understood that they can be administered in any order to a subject in need thereof. For example, a present HDAC6I can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent treatment modality (e.g., radiotherapy), to an individual in need thereof. In various embodiments, a present HDAC6I and the second therapeutic agent are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, the components of the combination therapies are administered at 1 minute to 24 hours apart.

The use of the terms “a”, “an”, “the”, and similar referents in the context of describing the invention (especially in the context of the claims) are to be construed to cover both the singular and the plural, unless otherwise indicated. Recitation of ranges of values herein merely serve as a shorthand method of referring individually to each separate value falling within the range, unless otherwise indicated herein, and each separate value and subrange is incorporated into the specification as if it were individually recited herein. The use of any and all examples, or exemplary language (e.g., “such as” and “like”) provided herein, is intended to better illustrate the invention and is not a limitation on the scope of the invention unless otherwise claimed. No language in the specification should be construed as indicating any non-claimed element as essential to the practice of the invention.

### EXAMPLES

The invention now being generally described, it will be more readily understood by reference to the following examples which are included merely for purposes of illustration of certain aspects and embodiments of the present invention and are not intended to limit the invention.

### Example 1: Synthesis of Exemplary Compounds of the Disclosure

### Synthetic Methods and Procedures

The central (hetero-)aromatic linker moiety is commercially available in a variety of structural modifications, such as:

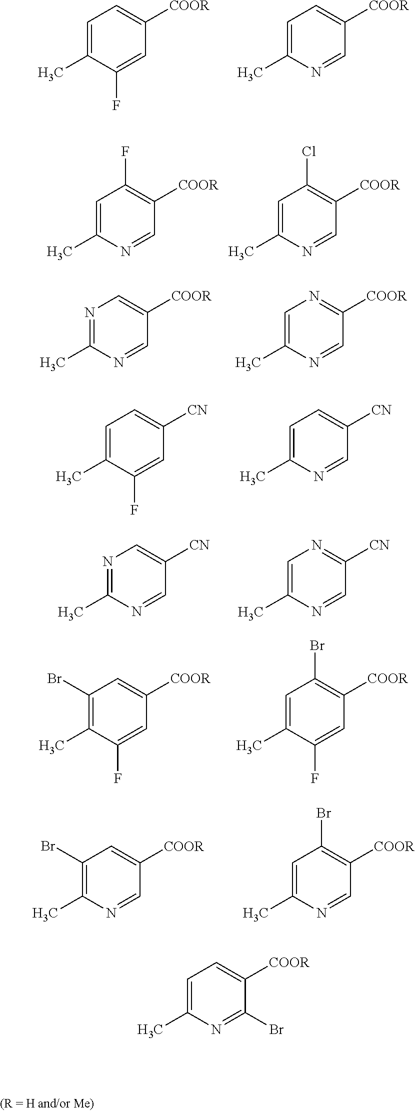

Carboxylic acids, their methyl esters, and their nitriles are furthermore interconvertable by standard synthetic methods, or nitriles may be obtained from (hetero-)aryl halides by transition metal catalyzed cyanation. Brominated building blocks are useful for the modification of these linker moieties at later stages of the synthesis through organometallic reactions, in particular transition-metal catalyzed coupling reactions. For simplicity, building blocks are in the following drawn without the substituents R₂. Analogous reactions are employed for intermediates where these substituents are present.

To prepare 1,3,4-oxadiazoles, hydrazides are obtained either from carboxylic acid esters by hydrazinolysis, or from free carboxylic acids by coupling with a protected hydrazine under the action of an amide/peptide coupling reagent, e. g., of the carbodiimide type, preferably with addition of an activating agent, such as 4-(dimethylamino)pyridine (DMAP). Protecting groups for hydrazine include, for example, tert-butoxycarbonyl (Boc), which is subsequently removed by mild acid treatment, e. g., by warming in hexafluoroisopropanol as solvent, or benzyloxycarbonyl (Cbz), which can be removed under a variety of conditions including catalytic hydrogenolysis over a Pd catalyst. Hydrazides are then acylated at their NH₂ group with di- or trifluoroacetic anhydride in the presence of a mild base. The resulting mixed 1,2-diacylhydrazines can be isolated and dehydrated to the desired 1,3,4-oxadiazoles in a separate step (e. g., with triflic anhydride and a base, or with CBr₄/PPh₃), or more conveniently are dehydrated under the conditions of their formation by an axcess of the acylating mixture.

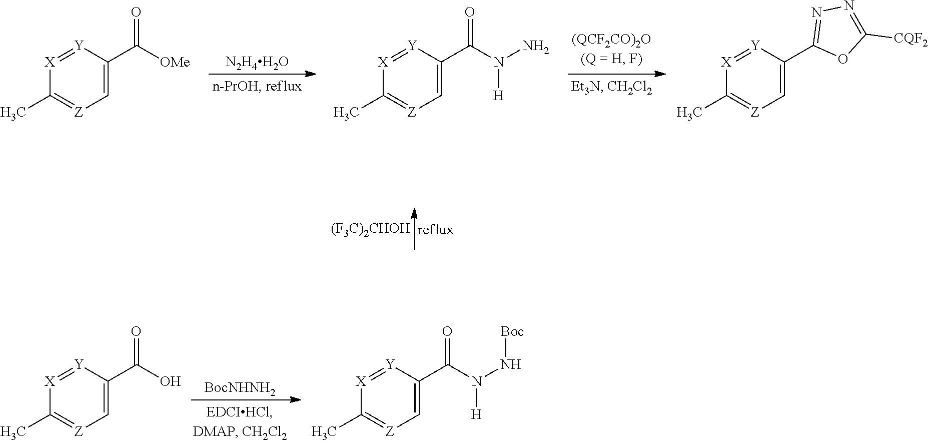

To prepare 1,2,4-oxadiazoles with the fluoroalkyl group residing in position 5, nitriles are converted with hydroxylamine to amidoximes. Amidoximes are N-acylated with di- or trifluoroacetic anhydride in the presence of a mild base. Use of an excess of the acylating mixture dehydrates the acylated amidoxime concomitantly with its formation to yield 3-(hetero-)aryl-5-(di- or trifluoromethyl)-1,2,4-oxadiazoles.

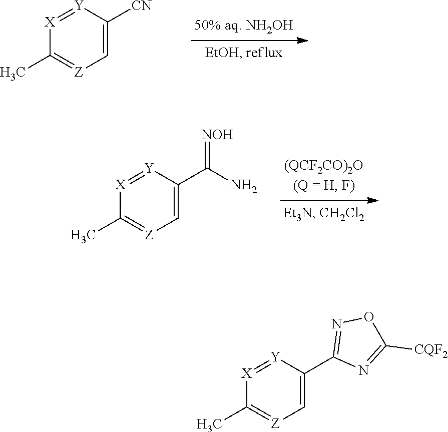

The inverse substitution pattern in the 1,2,4-oxadiazole series, i. e., fluorinated alkyl in position 3 and (hetero-)aryl in position 5, is accessible from N-acylated di- and trifluoroacetamidoximes. Trifluoroacetamidoxime is commercially available and can be N-acylated with (hetero-)aroyl halides in the presence of a weak base, such as pyridine, or with carboxylic acids in the presence of an amide/peptide coupling reagent. Cyclization of the resulting intermediates can, if not occurring spontaneously, be effected with dehydrating agents such as trifluoroacetic anhydride/base.

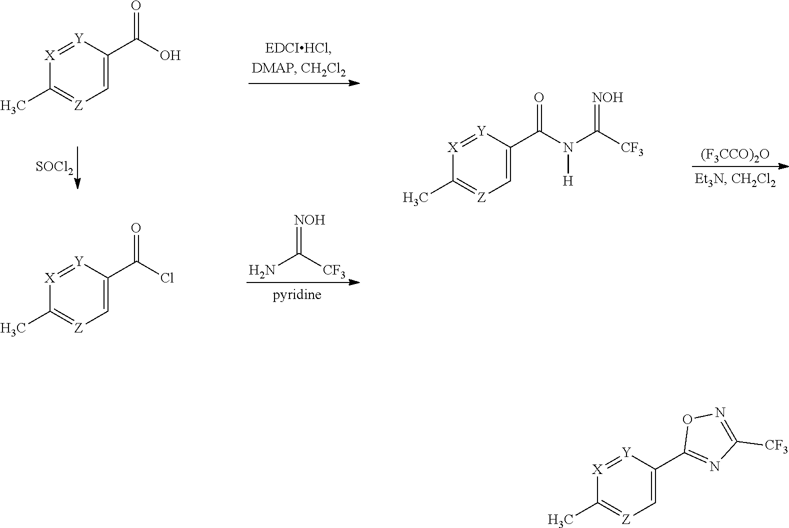

To prepare the analogous difluoromethylated 1,2,4-oxadiazoles, it may be advantageous to proceed through the 3-carboxaldehydes as intermediates, which are then deoxofluorinated with reagents commonly employed for this purpose, such as sulfur tetrafluoride, diethylaminosulfur trifluoride (DAST), bis(2-methoxyethyl)aminosulfur trifluoride (Deoxo-Fluor®), or N,N-diethyl-S,S-difluorosulfiliminium tetrafluoroborate (XtalFluor-E®). The 3-carboxaldehydes are obtained from their diethyl acetals in a sequence like the preceding one starting from 2,2-diethoxyacetamidoxime. This building block is in turn formed by addition of hydroxylamine to diethoxyacetonitrile, a commercially available liquid that is more convenient to handle than the gaseous difluoroacetonitrile required for the preparation of difluoroacetamidoxime.

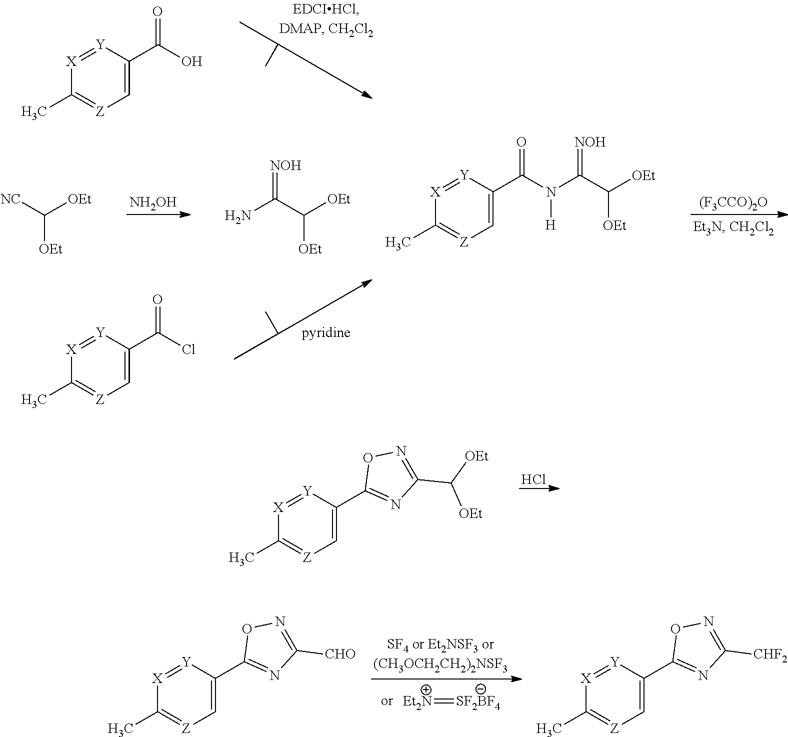

Oxazoles are commonly encountered in heterocyclic chemistry and can be prepared by a great variety of methods. The classical Robinson-Gabriel approach, consisting of the dehydration of 2-(acylamino)ketones, can be used to prepare certain compounds of the present invention, in which the (hetero-)aryl group is attached at the 5-position of the oxazole ring. Many dehydrating agents such as those given below effect this transformation. Acylaminoketones are prepared by N-acylation of aminoketones, for example in the form of their stable salts such as hydrochlorides. Aminoketone salts may be prepared from nitriles containing the requisite linker (hetero-)aromatic ring by a two-step procedure consisting of addition of a Grignard reagent to form an imine, followed by Neber rearrangement (Baumgarten, H. E. et al., J. Org. Chem. 1963, 28, 2369).

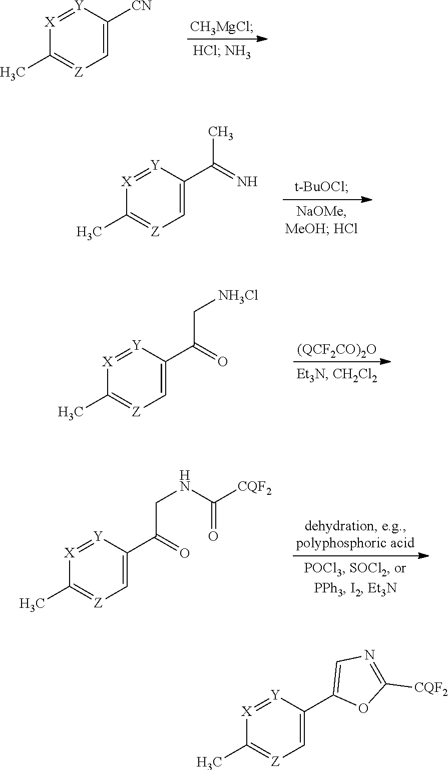

2-(Acylamino)ketone intermediates may also be obtained from diazoketones by Rh-catalyzed carbenoid formation and insertion into the N—H bond of a primary carboxamide, in the present case, di- or trifluoroacetamide (Davies, J. R. et al. Tetrahedron 2004, 60, 3967). Diazoketones can be synthesized from diazoalkanes and their derivatives by acylation with acyl halides in the presence of a mild base.

Oxazoles are amenable to bromination in 4-position with NBS in acetone (Khan, A. H.; Chen, J. S. Org. Lett. 2015, 17, 3718). The 4-bromide is a useful derivatives for the introduction of various substituents by transition metal catalyzed displacement and coupling reactions, e. g., trifluoromethylation by Cu-catalyzed reaction with methyl 2,2-difluoro-2-(fluorosulfonyl)acetate (Chen, Q. Y.; Wu, S. W. J. Chem. Soc., Chem. Commun. 1989, 705; Clarke, S. L.; McGlacken, G. P. Chem. Eur. J. 2017, 23, 1219). Methylation can be effected by Stille coupling with Me₄Sn; cyanation by a variety of protocols employing cyanide anion or various sources thereof, typically under Cu or Pd catalysis (example: Ren, Y. et al. Tetrahedron Lett. 2009, 50, 4595); fluorination by Br—F exchange under Pd catalysis (Lee, H. G. et al. J. Am. Chem. Soc. 2014, 136, 3792); and chlorination by Br—Cl exchange under Cu catalysis (Feng, X. et al. Chem. Commun. 2012, 48, 9468). The 4-carbonitriles resulting from cyanation can be reduced, e. g., with diisobutylaluminum hydride, to aldehydes, which are precursors to 4-(difluoromethyl)oxazoles by way of deoxofluorination (DAST etc.).

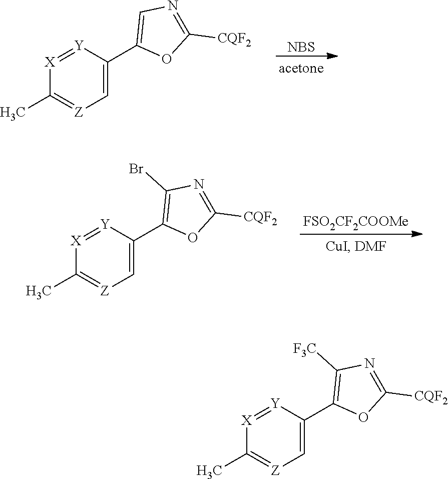

Oxazoles isomeric with the preceding series, i. e., with the (hetero-)aryl group attached to the ring's 4-position, are available, for example, through a recently reported oxidative procedure (Xiao, F. Org. Let. 2019, 21, 8533) from (hetero-)aryl methyl ketones. These starting materials can be prepared from carboxylic acids by way of the reaction of their derived Weinreb amides with methylmagnesium halides, or (not shown) by action of the same reagent on nitriles, followed by imine hydrolysis, some (for example, 5-acetyl-2-methylpyridine) are commercially available. The protocol places an arylthio substituent at the 5-position, which is removed by standard desulfurization with Raney nickel or nickel boride.

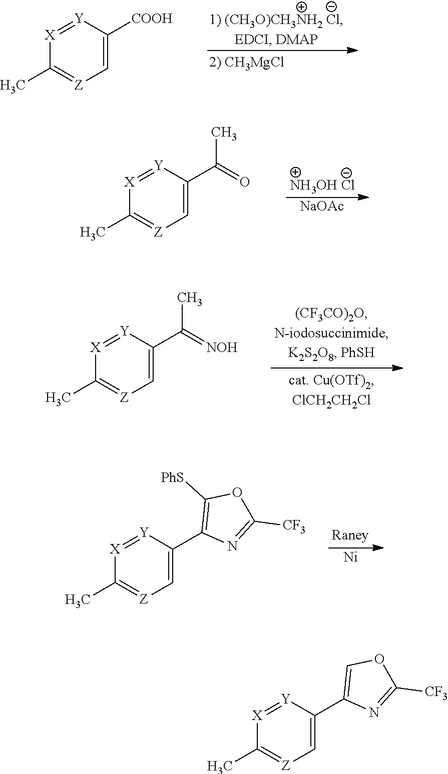

With the linker/zinc binding group partial structure assembled, the methyl group attached to the six-membered ring needs to be transformed into an alkylating agent. This can be effected through free radical bromination. The brominating agent typically is N-bromosuccinimide (NBS) or 1,3-dibromo-5,5-dimethylhydantoin, and the radical initiator an azo compound, such as azobis(isobutyronitrile) (AIBN), or a peroxide, such as dibenzoyl peroxide. These initiators have been widely employed for reactions conducted at the boiling temperature (77° C.) of carbon tetrachloride, the traditionally preferred solvent, at which temperature they have a convenient rate of decomposition. This environmentally hazardous solvent has in the present body of work been replaced with fluorobenzene, a nonpolar solvent of low reactivity and similar boiling point (85° C.). Photochemical initiation is also possible. In addition to the desired monobromination products, unreacted starting materials and dibromination products are also encountered. They are typically readily separable by normal-phase column chromatography. Dibrominated products can be recycled to the starting materials by free radical (e. g., Bu₃SnH or H₃PO₂/AIBN) or catalytic (e. g., H₂, Pd/C) hydrodebromination.

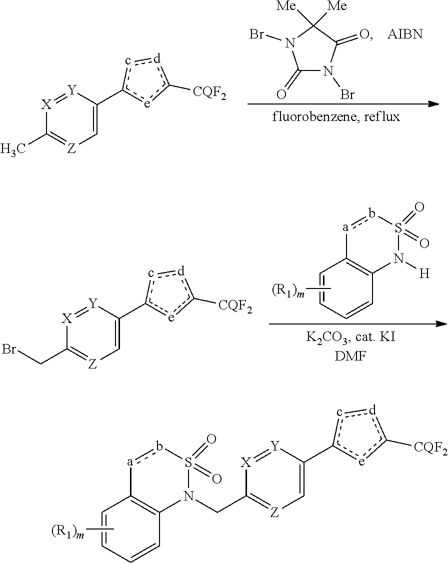

The sultam building block and the above alkylating agent are joined together by reaction in a dipolar-aprotic solvent, such as DMF, in the presence of a mild base. Methods for the preparation of the requisite sultams are disclosed in the following paragraphs.

3,4-Dihydro-1H-benzo[c][1,2]thiazine-2,2-dioxide and certain derivatives are accessible by a literature procedure (Moroda, A.; Togo, H. Synthesis 2008, 1257) that commences from 2-arylethanesulfonyl chlorides. Tolerated substituents include halo and alkyl. While the unsubstituted sulfochloride is commercially available, certain derivatives need to be synthesized, e. g., through the depicted sequence. The final reductive cleavage of the N—O bond can be effected with a variety of methods, such as reduction with SmI₂, catalytic hydrogenolysis, dissolving metal reduction protocols, and electrochemical methods. Incidental cleavage during the execution of certain other transformations has also been observed.

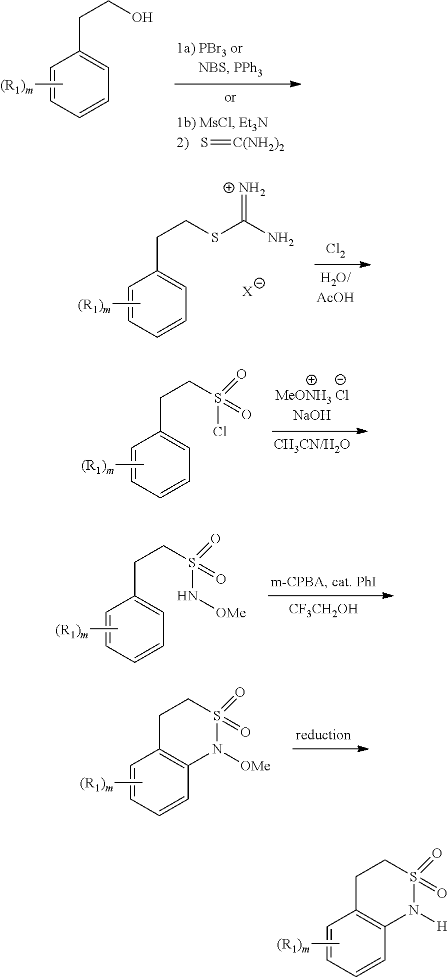

Ortho- and para-substituents in the 2-arylethanesulfonyl chloride unequivocally lead to 5- and 7-substituted 3,4-dihydro-1H-benzo[c][1,2]thiazine-2,2-dioxides, respectively, while meta substituents can lead to mixtures of 6- and 8-substituted compounds that need to be separated. A preferred entry into the 6-substituted series is electrophilic substitution on intermediates lacking a 6-substituent, in which the nitrogen atom exerts a predominantly or wholly para-directing influence. An example is the 6-bromination with N-bromosuccinimide (NBS) of either the N-methoxy precursor or the free sultam in the parent series (all R₁═H).

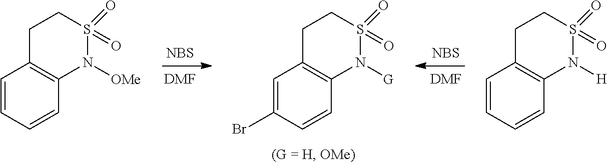

The same general sequence is applicable to compounds in which the aliphatic carbon atoms bear substituents. The required substituted phenethyl alcohols are accessible, for example, by reduction of substituted phenylacetic acids or their esters. The latter can be synthesized, for example, by alkylation of the ester enolates, or by their arylation under transition metal catalysis. An example is shown below, in which the ester enolate is a zinc enolate formed by Reformatsky reaction of the corresponding α-bromo ester with Zn metal, and the arylation step is a Negishi coupling reaction (Sakuma, D. et al. Chem. Lett. 2015, 44, 818).

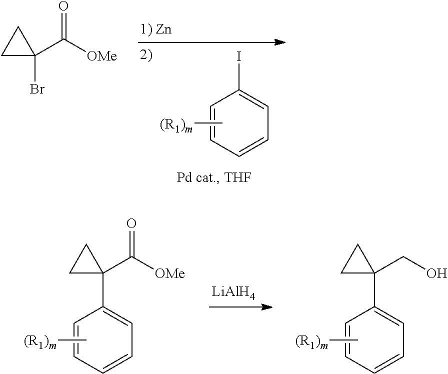

3,4-Dihydro-1H-benzo[c][1,2]thiazine-2,2-dioxides bearing a halogen substituent, such as Cl and Br, on the aromatic ring, are suitable starting materials for the introduction of other substituents by transition metal catalyzed exchange and coupling reactions. This approach is illustrated by the example of a Suzuki coupling reaction between the 3,4-dihydro-1-methoxy-1H-benzo[c][1,2]thiazine-2,2-dioxide and an arylboronic acid, in which the predominant product is not the simple coupling product but instead the product of coupling and concomitant reductive cleavage of the N—O bond. Other possibilities include but are not limited to halogen-halogen exchange, alkoxylation, amination, amidation, trifluoromethylation, trifluoromethylthiolation, cyanation, and carbonylation reactions, as well as Heck, Kumada, Negishi, Sonogashira, and Stille coupling reactions. Such reactions have seen much emphasis in modern synthetic methodology research and are familiar to those skilled in the art.

3,4-Dihydro-1H-benzo[c][1,2]thiazine-2,2-dioxides bearing a halogen substituent, such as Cl and Br, on the aromatic ring, are suitable starting materials for the introduction of other substituents by transition metal catalyzed exchange and coupling reactions. This approach is illustrated by the example of a Suzuki coupling reaction between the 3,4-dihydro-1-methoxy-1H-benzo[c][1,2]thiazine-2,2-dioxide and an arylboronic acid, in which the predominant product is not the simple coupling product but instead the product of coupling and concomitant reductive cleavage of the N—O bond. Other possibilities include but are not limited to halogen-halogen exchange, alkoxylation, amination, amidation, trifluoromethylation, trifluoromethylthiolation, cyanation, and carbonylation reactions, as well as Heck, Kumada, Negishi, Sonogashira, and Stille coupling reactions. Such reactions have seen much emphasis in modern synthetic methodology research and are familiar to those skilled in the art.

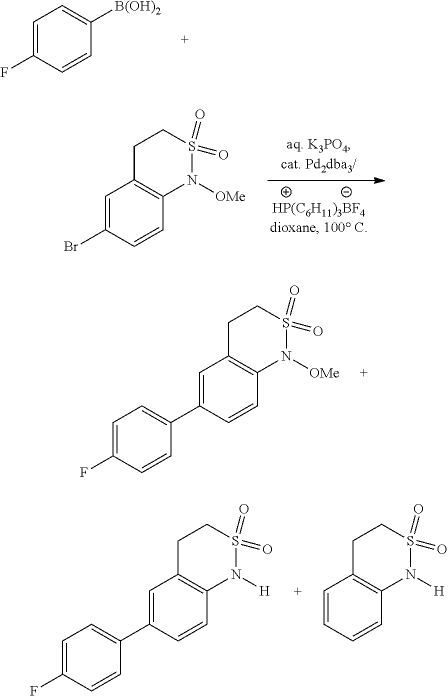 [SMILES: OB(O)c1ccc(F)cc1 ; CON1c2ccc(Br)cc2CCS1(=O)=O ; CON1c2ccc(-c3ccc(F)cc3)cc2CCS1(=O)=O ; O=S1(=O)CCc2cc(-c3ccc(F)cc3)ccc2N1 ; O=S1(=O)CCc2ccccc2N1]

If neither a 1-methoxysultam nor the corresponding free sultam are suitable for an intended reaction, e. g., because of premature loss of the 1-methoxyl group or because of the N—H acidity or limited solubility of the free sultam, a different N-protecting group may be introduced and later removed when no longer needed. An example is the methoxymethyl group, which can be introduced under mild alkylation conditions and removed by treatment with acid. Other suitable protecting groups include but are not limited to benzyloxymethyl, which offers the added option of hydrogenolytic cleavage; silyl groups, such as tert-butyldimethylsilyl, ter-butyldiphenylsilyl, and triisopropylsilyl, which are removed by treatment with acids, protic bases, or fluoride anion; benzyl and benzyloxycarbonyl, which are removed by hydrogenolysis; tert-butoxycarbonyl, which is removed with acid; and 4-methoxybenzyl and 2,4-dimethoxybenzyl, which are removed by oxidation or by treatment with strong acids.

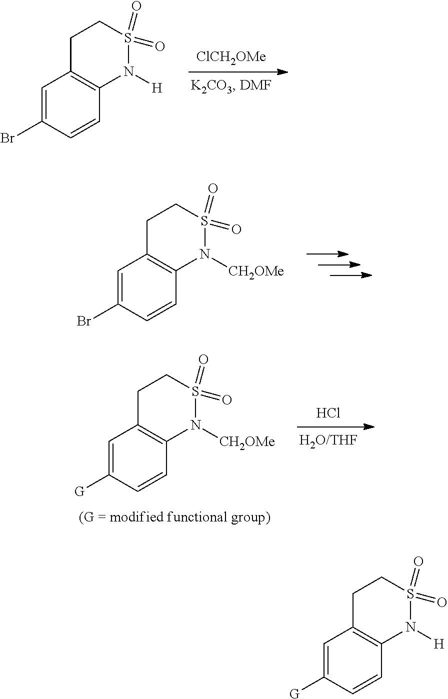

4-Keto-derivatives, i. e., 1H-benzo[c][1,2]thiazin-4(3H)-one 2,2-dioxides, can be synthesized according to the following scheme, which is analogous to the work of Shafiq, M. et al. J. Chil. Chem. Soc. 2011, 56, 527, with N-Boc replacing N-Me to permit access to the N-unsubstituted sultams.

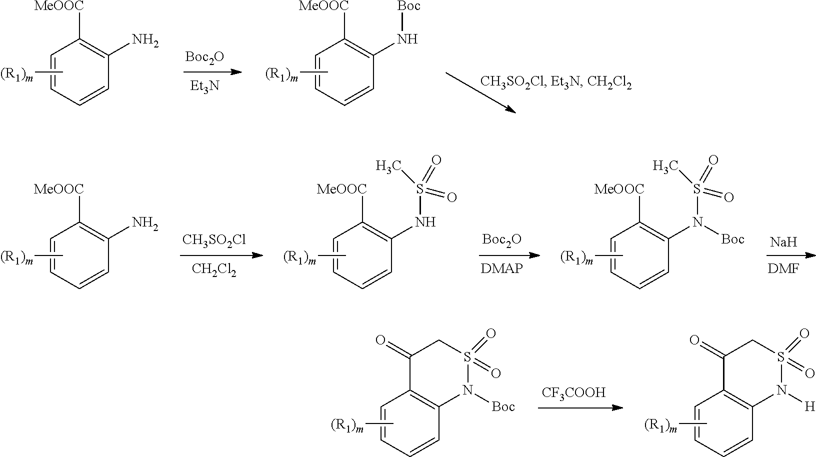

Another method for the preparation of 1H-benzo[c][1,2]thiazin-4(3H)-one 2,2-dioxides consists in the N-sulfonylation of anilines with methoxycarbonylmethanesulfochloride. After ester hydrolysis, the carboxylic acid is cyclized in an intramolecular Friedel-Crafts acylation reaction (Pasteris, R. J. U.S. Pat. No. 4,867,781, Sep. 19, 1989; Nie, H.; Widdowson, K. L. U.S. Pat. No. 6,436,927, Aug. 20, 2002).

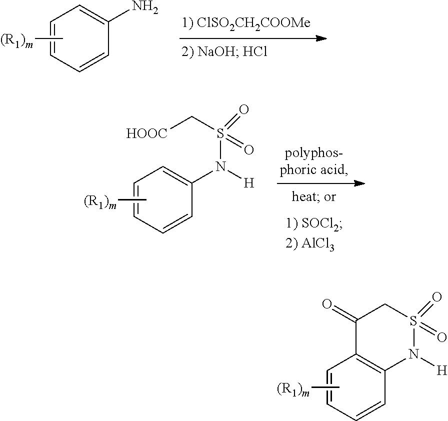

1H-Benzo[c][1,2]thiazin-4(3H)-one 2,2-dioxides react with tosylhydrazine to form tosylhydrazones, whose reduction with catecholborane constitutes an alternative synthesis of 3,4-dihydro-1H-benzo[c][1,2]thiazine 2,2-dioxides (Pasteris, R. J. U.S. Pat. No. 4,867,781, Sep. 19, 1989). Upon treatment with bases, the tosylhydrazones undergo a Bamford-Stevens reaction to furnish 11H-benzo[c][1,2]thiazine 2,2-dioxides (Nie, H.; Widdowson, K. L. U.S. Pat. No. 6,436,927, Aug. 20, 2002).

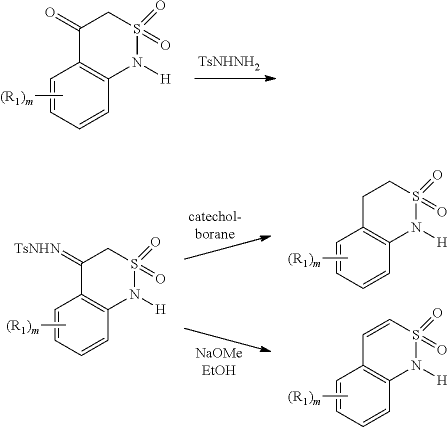

Sultam building blocks containing a heteroatom in position 4 are accessible from o-phenylenediamines, o-aminophenols, and o-mercaptoanilines by reaction with chloromethanesulfonyl chloride in the presence of a base. In the case of X═NH, oxidation with MnO₂ delivers the corresponding unsaturated heterocycle (Nie, H.; Widdowson, K. L. U.S. Pat. No. 6,436,927, Aug. 20, 2002).

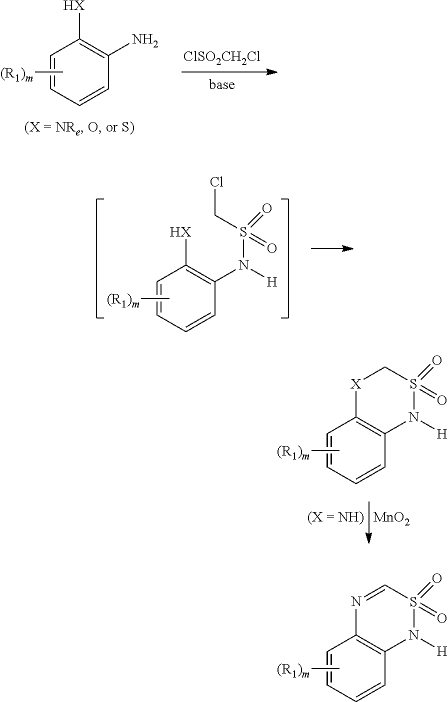

3,4-Dihydro-1H-benzo[c][1,2,6]thiadiazine 2,2-dioxides are formed upon heating of 2-aminobenzylamines with sulfamide in pyridine (CN1909909B, 2010 Dec. 15). One way of synthesizing the requisite diamine intermediates is through oxime formation from 2-nitrophenyl alkyl ketones followed by reduction, e. g., by catalytic hydrogenation.

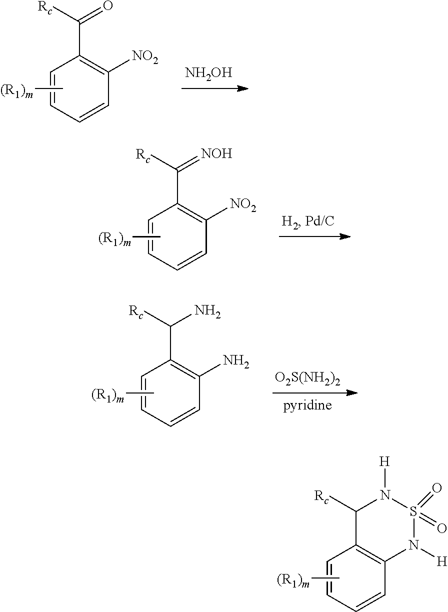

Up to this paragraph, the compounds of the invention are synthesized by first constructing the zinc-binding fluoroalkylated oxadiazole or oxazole moiety onto the linker ring and then connecting this assembly to the sultam cap group. It is also possible to first connect the cap group to the linker and then to build the fluoroalkylated oxadiazole or oxazole moiety onto that assembly, utilizing the same types of reactions as in the first-disclosed approach. Possible functional group incompatibilities can be remedied by use of appropriate protecting groups or by generating the interfering functional group in a later step from a less reactive precursor, e. g., an aromatic bromide, by reactions such as displacement or coupling reactions.

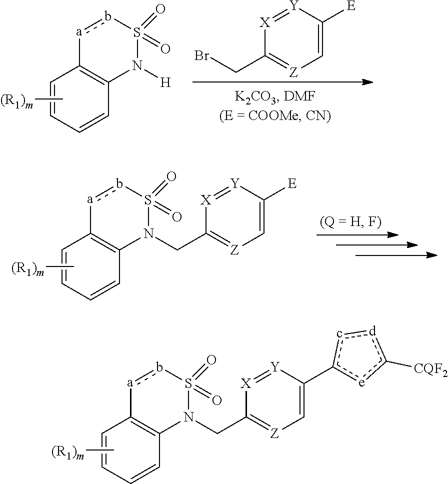

The following synthetic schemes are representative of the reactions used to synthesize the present HDAC6Is. Modifications and alternate schemes to prepare HDAC6Is of the invention are readily within the capabilities of persons skilled in the art.

All starting materials and solvents were purchased from commercial suppliers at reagent purity and were used as obtained without further purification. Reactions were monitored by thin layer chromatography on silica gel-coated glass plates with visualization at 254 nm and/or using appropriate stains. ¹H NMR spectra were recorded at 400 or 500 MHz on Bruker Avance-400 and Avance-500 spectrometers, respectively. Chemical shifts (S scale) are reported in parts per million (ppm) relative to TMS (in CDCl₃) or relative to the solvent signal (in DMSO-da; solvent signal set to S 2.50). Signals are characterized as: s (singlet), d (doublet), t (triplet), m (multiplet), and br (broad).

### Compound 1. 1-[[5-[5-(Difluoromethyl)-1,3,4-axadiazol-2-yl)pyridin-2-yl]methyl]-3,4-dihydro-1H-benzo[c][1,2]thiazine-2,2-dioxide

### N-(6-Methylnicotinoyl)hydrazine

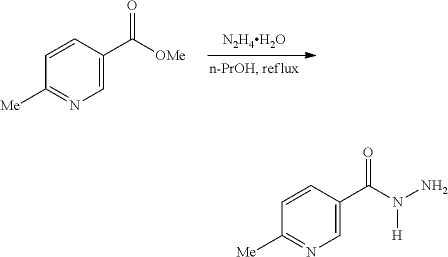 [SMILES: COC(=O)c1ccc(C)nc1 ; "N2H4·H2O" ; Cc1ccc(C(=O)NN)cn1]

In a 500 mL round-bottom flask with stir bar, heating mantle, reflux condenser, and Ar balloon, a mixture of methyl 6-methylnicotinate (7.39 g, 48.9 mmol), 1-propanol (60 mL), and hydrazine monohydrate (9.5 mL, 195 mmol, 4.0 equiv.) was heated to reflux for 22 h. TLC analysis (SiO₂, MeOH/CHCl₃) showed the hydrazide at R_{f} approx. 0.25 as the only significant component. The mixture was evaporated, and the residue was taken up in MeOH and adsorbed on SiO₂ (50 g). The residue was chromatographed on SiO₂ (15×6 cm) starting with MeOH/CH₂Cl₂ 1:9, then changing to MeOH/CH₂Cl₂ 15:85. At the appearance of the hydrazide in the eluate, the mixing ratio was further changed to 1:4. The product-containing eluate was concentrated to a small volume, and toluene (50 mL) was added to ensure complete removal of MeOH. The evaporation was completed and the residue dried under vacuum to yield 6.83 g (92%) of a colorless solid. ¹H NMR (400 MHz, DMSO-d₆) δ 9.86 (s, 1H), 8.84 (narrow m, 1H), 8.04 (dd, 1H, J=8.1, 2.4 Hz), 7.33 (d, 1H, J=8.1 Hz), 4.52 (s, 2H); CH₃ concealed by the DMSO-d₅ signal. ¹H NMR (400 MHz, D₂O) δ 8.69 (narrow m, 1H), 8.02 (dd, 1H, J=8.2, 2.4 Hz), 7.41 (d, 1H, J=8.2 Hz), 2.56 (s, 3H).

### 5-[5-(Difluoromethyl)-1,3,4-oxadiazol-2-yl]-2-methylpyridine

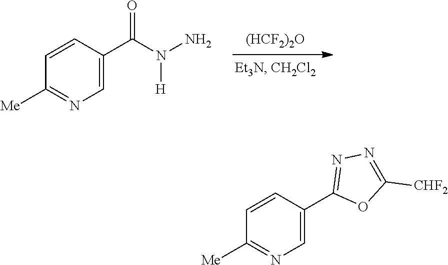

In a 500 mL round-bottom flask with stir bar, dropping funnel, and balloon were placed N-(6-methylnicotinoyl)hydrazine (6.83 g, 45.2 mmol), anhydrous CH₂Cl₂ (80 mL), and triethylamine (31.5 mL, 226 mmol, 5.0 equiv.). The suspension was cooled in an ice bath, and difluoroacetic anhydride (16.0 mL, 136 mL, 3.0 equiv.) was added dropwise in 45 min. The starting material soon dissolved. The solution was stirred at ambient temperature for 22.5 h, and it turned very dark. TLC (SiO₂, EtOAc/hexane 1:1) showed the product at R_{f} approx. 0.35 together with a diffuse polar tail and a brown baseline spot. Water (10 mL) was added dropwise with water cooling in 20 min, followed by a solution of citric acid (17.3 g, 90 mmol, 2.0 equiv.) in water (40 mL). The pH of the aqueous phase was approx. 3.5. The phases were separated, and the aqueous phase was sequentially extracted with CH₂Cl₂ and EtOAc (50 mL each). The combined organic phases were washed with brine (50 mL; organic phase=lower) and evaporated to leave a dark-brown oil, which was chromatographed on SiO₂ (20×5 cm, EtOAc/hexane 1:1). Most of a forerun (not seen on the bulk TLC) was removed, but some of the polar tail stayed with the product. Evaporation left 6.9 g of a brown oil, which solidified on standing. This crude material was twice bulb-to-bulb distilled (105-125° C./oil pump vacuum) to furnish 6.17 g of a mixture of colorless crystals and a yellow oil. The distillate was taken up in MeOH (20 mL), and the solution was placed in a freezer. Initially no crystallization occurred. A drop of the solution was allowed to evaporate, whereon crystals formed. These were used to seed the remaining solution, from which the product then crystallized rapidly. After another 3 h in the freezer, the product was isolated by suction filtration, washed with cold MeOH (10 mL), and dried under vacuum to yield 3.77 g (40%) of colorless crystals, contaminated (according to ¹H NMR) with approx. 3 mol % of difluoroacetic acid. ¹H NMR (CDCl₃, 400 MHz) δ 9.22 (narrow m, 1H), 8.29 (dd, 1H, J=8.2, 2.3 Hz), 7.37 (d, 1H, J=8.2 Hz), 6.95 (t, 1H, J_{H-F}=51.7 Hz), 2.69 (s, 3H); difluoroacetic acid at S 5.97 (t, 1H, J_{H-F}=54.7 Hz). ¹³C NMR (CDCl₃, 100 MHz) δ 164.44, 163.27, 158.32 (t, J_{C-F}=29.2 Hz), 147.77, 134.85, 123.61, 116.48, 105.68 (t, J_{C-F}=241.2 Hz), 24.79.

The mother liquor contained much difluoroacetic acid (by NMR). It was evaporated and taken up in fresh MeOH (30 mL). Amberlite IRA-67 (weakly basic anion exchange resin, free base; Sigma No. A9960; CAS registry number 65899-87-7; 4 g) was added. The mixture was swirled from time to time for 50 min. The resin was removed by suction filtration and washed with MeOH (3×5 mL). An aliquot of the filtrate was analyzed by ¹H NMR, and a small concentration of difluoroacetic acid was found to have persisted. The treatment with anion exchange resin (2 g) was repeated for 5.7 h. After filtration from the resin, the filtrate was adsorbed on SiO₂ (8 g). The residue was chromatographed on SiO₂ (19×3.8 cm, t-BuOMe; TLC, R_{f} approx. 0.5, slightly streaking) to remove some polar material. The eluate was evaporated and dried under vacuum to yield another 1.24 g (13%) of the oxadiazole as yellowish crystals.

### 2-(Bromomethyl)-5-[5-(difluoromethyl)-1,3,4-oxadiazol-2-yl]pyridine

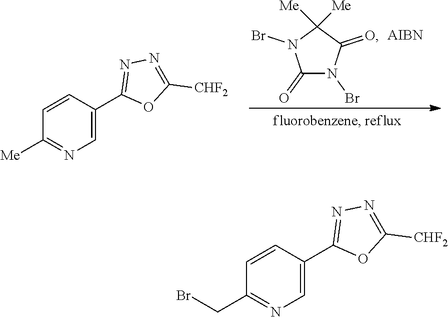 [SMILES: Cc1ccc(-c2nnc(C(F)F)o2)cn1 ; CC1(C)C(=O)N(Br)C(=O)N1Br ; FC(F)c1nnc(-c2ccc(CBr)nc2)o1]

In a 250 mL 3-necked flask with stir bar, heating mantle, stopper, reflux condenser (connected to an Ar balloon), and dropping funnel with septum were placed 5-[5-(difluoromethyl)-1,3,4-oxadiazol-2-yl]-2-methylpyridine (3.75 g, 17.8 mmol), 1,3-dibromo-5.5-dimethylhydantoin (2.78 g, 9.8 mmol, 0.55 equiv.), and fluorobenzene (50 mL). The atmosphere was replaced with Ar. The dropping funnel was charged with a solution of azobis(isobutyronitrile) (AIBN; 0.44 g, 2.7 mmol, 0.15 equiv.) in fluorobenzene (5 mL), of which approx. 20% was added to the reaction mixture. The mixture was heated to reflux, and the remainder of the AIBN solution was added in small portions over the course of 1 h. At this point, most of the color of the temporarily formed Br₂ had faded. Reflux was continued for 10 min, and the mixture was cooled to ambient temperature. TLC (SiO₂, EtOAc/hexane 2:3) showed three major components at R_{f} approx. 0.75, 0.5, and 0.3, which corresponded to the dibromination byproduct, the desired monobromination product, and starting material, respectively. The dark solution was filtered from the precipitated hydantoin with suction over celite, and the fluorobenzene was recovered by rotary evaporation. The hydantoin was washed with the recovered fluorobenzene, and the combined liquids were again evaporated. The residue was chromatographed on SiO₂ (29×5 cm, EtOAc/hexane 1:3 for the dibromide, 1:2 for the monobromide, and 3:2 for the starting material). Evaporation of the appropriate fractions yielded 1.34 g (semisolid; 20%) of the dibromide, 2.55 g (off-white solid; 49%) of the monobromide, and 1.07 g (light-amber solid, 29%) of the starting material. Monobromide: ¹H NMR (400 MHz, CDCl₃) δ 9.29 (narrow m, 1H), 8.42 (dd, 1H, J=8.2, 2.2 Hz), 7.66 (dd, 1H, J=8.2, 0.4 Hz), 6.95 (t, 1H, J_{H-F}=51.6 Hz), 4.62 (s, 2H).

### N-Methoxy-2-phenylethanesulfonamide

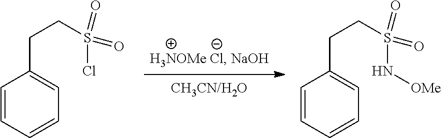 [SMILES: O=S(=O)(Cl)CCc1ccccc1 ; CONS(=O)(=O)CCc1ccccc1]

In a 250 mL round-bottom flask with stir bar and dropping funnel, O-methylhydroxylamine hydrochloride (5.2 g, 62 mmol, 2.2 equiv.) was suspended in CH₃CN (40 mL). The mixture was cooled in an ice bath, and a solution of NaOH (2.5 g, 62 mmol, 2.2 equiv.) in water (15 mL) was added dropwise in 10 min. After another 25 min at 0° C., the dropping funnel was replaced with a dry one for the dropwise addition over 15 min of a solution of 2-phenylethanesulfonyl chloride (5.75 g, 28.1 mmol) in anhydrous CH₃CN (20 mL). Stirring was continued for 20 min in the ice bath, then at ambient temperature for 22 h. After partial evaporation to remove the organic solvent (frothing!), water (80 mL) was added, and the product was extracted into EtOAc (3×50 mL). The combined organic phases were dried over Na₂SO₄ (20 g) and evaporated, and the residue was dried under vacuum to yield 5.93 g (98%) of the sulfonamide as a colorless solid. ¹H NMR (CDCl₃, 400 MHz) δ 7.37-7.32 (m, 1H), 7.31-7.23 (m, 3H), 6.63 (br s, 1H), 3.77 (s, 3H), 3.51, 3.13 (AA′XX′ multiplet, high-field part slightly broadened, 4H, J_{AX}+J_{AX′}=15.6 Hz).

### 3,4-Dihydro-1-methoxy-1H-benzo[c][1,2]thiazine-2,2-dioxide

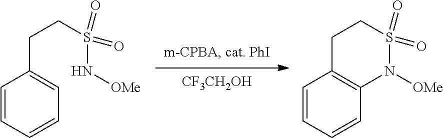 [SMILES: CONS(=O)(=O)CCc1ccccc1 ; "CF3CH2OH" ; CON1c2ccccc2CCS1(=O)=O]

In a 500 mL round-bottom flask with stir bar, O-methoxy-2-phenylethanesulfonamide (5.98 g, 27.8 mmol) was dissolved with gentle warming in 2,2,2-trifluoroethanol (83 mL). Iodobenzene (0.31 mL, 2.8 mmol, 0.10 equiv.) was added. m-Chloroperoxybenzoic acid (75%, remainder m-chlorobenzoic acid and water; 7.05 g, 30.6 mmol, 1.1 equiv.) was added portionwise in 10 min, resulting in a mild exotherm. The flask was loosely stoppered, and the mixture was stirred without temperature control for 4 h. Early on, the peroxyacid went into solution; later, a pale-yellow, then tan suspension formed. Shortly before quenching, TLC analysis (SiO₂, EtOAc/toluene 1:9) showed the product at R_{f} approx. 0.5, iodobenzene near the solvent front, baseline material, and a polar streak. The mixture was stirred at rt for 25 min with a solution of Na₂SO₃ (2.5 g, 20 mmol) in water (50 mL). Evaporation of the organic solvent (bumping!) left a mostly solid residue, which was taken up in EtOAc (50 mL) and brine (30 mL). The phases were separated (somewhat slowly but completely), and the aqueous phase was extracted with EtOAc (2×50 mL). The pale-yellow combined organic phases were dried over Na₂SO₄ (20 g) and evaporated to near dryness. The residual oil was taken up in MeOH (50 mL), whereon rapid crystallization ensued. After standing overnight in the freezer, the precipitate was isolated by suction filtration, washed with cold MeOH (2×10 mL), and dried under vacuum to yield 4.25 g of colorless crystals. The mother liquor still contained, by TLC, product besides polar and nonpolar impurities, and was adsorbed on SiO₂ (5 g). Chromatography of the residue on SiO₂ (20×4 cm, EtOAc/toluene 1:19, then 1:9) yielded, after evaporation and drying under vacuum, another 0.90 g of the cyclization product in form of a colorless solid. Total yield: 5.15 g (87%). ¹H NMR (CDCl₃, 400 MHz) δ 7.40-7.36 (m, 1H), 7.36-7.29 (m, 2H), 7.23-7.19 (m, 1H), 4.08 (s, 3H), 3.50, 3.42 (AA′BB′ multiplet, high-field part slightly broadened, 4H).

### 3,4-Dihydro-1H-benzo[c][1,2]thiazine-2,2-dioxide

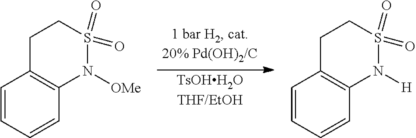 [SMILES: CON1c2ccccc2CCS1(=O)=O ; "TsOH·H2OTHF/EtOH" ; O=S1(=O)CCc2ccccc2N1]

A 250 mL round-bottom flask with stir bar was charged with 3,4-dihydro-1-methoxy-1H-benzo[c][1,2]thiazine-2,2-dioxide (2.05 g, 9.61 mmol), p-toluenesulfonic acid monohydrate (1.83 g, 9.6 mmol), 20% Pd(OH)₂/C (50% H₂O; Alfa Aesar No. 42578; 0.22 g), THF (20 mL), and EtOH (non-denatured, 40 mL). A H₂ balloon was placed on the flask by way of a three-way stopcock, and the flask was evacuated and flushed with H₂ three times. The hydrogenation was allowed to proceed at rt for 6.5 h, then the H₂ atmosphere was replaced with Ar. TLC analysis (SiO₂, EtOAc/hexane 1:1) showed a single spot for the product (R_{f} approx. 0.6), which was adequately separated from the starting material (R_{f} approx. 0.7). The mixture was adsorbed on SiO₂ (12 g), and the residue was filtered over SiO₂ (12×3 cm, EtOAc/hexane 43:57). The product-containing eluate was contaminated with some baseline material and furnished a pinkish semisolid on evaporation. This material was evaporated with MeOH to obtain a crystalline solid, which was taken up in tert-butyl methyl ether. Suction filtration, repeated washing with tert-butyl methyl ether, and drying under vacuum yielded 1.27 g (72%) of the product as an off-white powder of adequate purity (by ¹H NMR) to be used in further steps. ¹H NMR (DMSO-d₆, 400 MHz) δ 10.09 (s, 1H), 7.19 (d, 1H, J=7.4 Hz), 7.16 (t with unresolved long-range coupling, 1H, J=7.5 Hz), 6.96 (dt, 1H, J=1.1 Hz (d), 7.5 Hz (t)), 6.76 (dd, 1H, J=8.0, 0.8 Hz), 3.36-3.27 (AA′BB′ multiplet, 4H). ¹H NMR (CDCl₃, 400 MHz) δ 7.23-7.16 (m, 2H), 7.05 (dt, 1H, J=0.7 Hz (d), 7.5 Hz (t)), 6.75 (d, 1H, J=8.0 Hz), 6.37 (br s, 1H), 3.49, 3.32 (AA′XX′ multiplet, low-field part slightly broadened, 4H, J_{AX}+J_{AX′}=13.5 Hz).

### 1-[[5-[5-(Difluoromethyl)-1,3,4-oxadiazol-2-yl)pyridin-2-yl]methyl]-3,4-dihydro-1H-benzo[c][1,2]thiazine-2,2-dioxide

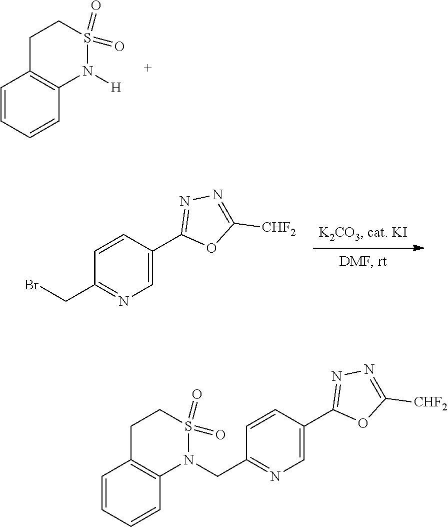 [SMILES: O=S1(=O)CCc2ccccc2N1 ; FC(F)c1nnc(-c2ccc(CBr)nc2)o1 ; O=S1(=O)CCc2ccccc2N1Cc1ccc(-c2nnc(C(F)F)o2)cn1]

In a 100 mL round-bottom flask with stir bar were placed 3,4-dihydro-1H-benzo[c][1,2]thiazine-2,2-dioxide (615 mg, 3.36 mmol), 2-[6-(bromomethyl)pyridin-3-yl]-5-(difluoromethyl)-1,3,4-oxadiazole (1.022 g, 3.52 mmol, 1.05 equiv.), finely ground K₂CO₃ (0.56 g, 4.05 mmol, 1.2 equiv.), KI (0.11 g, 0.67 mmol, 0.2 equiv.), and anhydrous DMF (10 mL). The flask was wrapped in Al foil for light protection, and the mixture was stirred at ambient temperature for 19 h. Water (200 mL) and EtOAc/hexane 1:1 (100 mL, then 2×50 mL) were added, and the phases were separated. The combined organic phases were washed with water (200 mL) and brine (25 mL). TLC (SiO₂, EtOAc/hexane 2:3) showed the product at R_{f} approx. 0.3. The solution was adsorbed on SiO₂ (10 g) and the material chromatographed on SiO₂ (9×5 cm, EtOAc/hexane 2:3). Evaporation of the product-containing fractions, evaporation of the residue with CH₃CN, and drying under vacuum yielded the product (1.28 g) as a colorless glass containing 2.4 weight % of CH₃CN (by ¹H NMR; corrected yield: 1.25 g, 95%). ¹H NMR (CDCl₃, 400 MHz) δ 9.30 (narrow m, 1H), 8.37 (dd, 1H, J=8.3, 2.2 Hz), 7.75 (d, 1H, J=8.3 Hz), 7.19 (d, 1H, 1=7.2 Hz), 7.13 (dt, 1H, J=1.2 Hz (d), 7.8 Hz (t)), 6.94 (t, 1H, J_{H-F}=51.7 Hz), 6.79 (d, 1H, J=8.3 Hz), 5.23 (s, 2H), 3.56, 3.52 (AA′BB′ multiplet, 4H).

### Compound 9. 1-[[5-[5-(Difluoromethyl)-1,3,4-oxadiazol-2-yl)pyridin-2-yl]methyl]-6-(4-fluorophenyl)-3,4-dihydro-1H-benzo[c][1,2]thiazine-2,2-dioxide 6-Bromo-3,4-dihydro-1-methoxy-1H-benzo[c][1,2]thiazine-2,2-dioxide

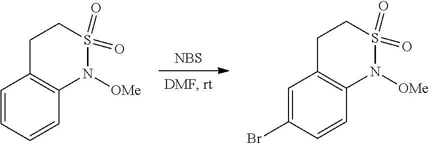

To a solution of 3,4-dihydro-1-methoxy-1H-benzo[c][1,2]thiazine-2,2-dioxide (0.95 g, 4.45 mmol) in DMF (8 mL) was added all at once N-bromosuccinimide (NBS; 0.79 g, 4.45 mmol). The mixture was stirred at rt for 21 h. TLC (micro-aqueous workup: water/EtOAc; SiO₂, EtOAc/hexane 1:4) showed comparable concentrations of the starting material (R_{f} approx. 0.25) and the product (R_{f} approx. 0.3). Another portion of NBS (0.40 g, 2.25 mmol, 0.5 equiv.) was added, and the bromination was allowed to proceed for another 44 h. TLC at this point indicated complete conversion. A solution of Na₂SO₃ (1 g) in water (20 mL) was added, and the mixture was stirred at rt for 20 min. More water (80 mL) and EtOAc/hexane 1:1 (50 mL) were added. The phases were separated, and the aqueous phase was extracted with EtOAc/hexane 1:1 (20 mL). The combined organic phases were washed with water (2×100 mL), dried over Na₂SO₄ (5 g), and concentrated to near dryness. MeOH (5 mL) was added, and the solution was placed in a freezer. Crystallization occurred slowly after scratching and was allowed to proceed for 2 days. The mother liquor was pipetted off, and the solid was washed with cold MeOH (5 mL). Drying under vacuum yielded 0.84 g (65%) of colorless material of 96% purity (by integration of the OMe ¹H NMR signals). Evaporation of the mother liquor left another 0.33 g (25%) of a light-amber glass, contaminated (by TLC) with traces of starting material but suitable for use in Suzuki coupling reactions. ¹H NMR (CDCl₃, 400 MHz) δ 7.44 (dd, 1H, J=8.5, 2.1 Hz), 7.37 (narrow m, 1H), 7.24 (d, 1H, J=8.6 Hz), 4.05 (s, 3H), 3.47, 3.37 (AA′XX′ multiplet, 4H, J_{AX}+J_{AX′}, =13.0 Hz).

### 6-(4-Fluorophenyl)-3,4-dihydro-1H-benzo[c][1,2]thiazine-2,2-dioxide

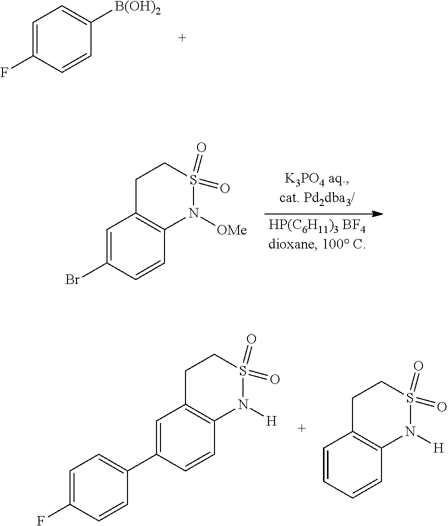 [SMILES: OB(O)c1ccc(F)cc1 ; CON1c2ccc(Br)cc2CCS1(=O)=O ; "HP(C6H11)3 BF4dioxane, 100° C." ; O=S1(=O)CCc2cc(-c3ccc(F)cc3)ccc2N1 ; O=S1(=O)CCc2ccccc2N1]

A resealable tube with stir bar was charged with 6-bromo-3,4-dihydro-1-methoxy-1H-benzo[c][1,2]thiazine-2,2-dioxide (332 mg, 1.14 mmol), 4-fluorophenylboronic acid (175 mg, 1.25 mmol, 1.1 equiv.), bis(dibenzylideneacetone)dipalladium(0) (14.5 mg, 16 μmol, 1.4%), tricyclohexyphosphonium tetrafluoroborate (10.5 mg, 28 μmol, 2.5%), K₃PO₄ (1.27M aqueous solution; 1.5 mL, 1.9 mmol, 1.7 equiv.), and dioxane (3.0 mL). The mixture was purged with Ar, and the vial was capped and heated to 100° C. (oil bath temperature) for 24 h. TLC (SiO₂, EtOAc/toluene 1:9) showed a major product at R_{f} approx. 0.2 and minor spots at R_{f} approx. 0.25 and 0.45. The phases were separated, and the aqueous phase was extracted with EtOAc (2×3 mL). The mixture was adsorbed on SiO₂ (3 g). The residue was chromatographed on SiO₂ (15×3 cm, EtOAc/toluene 1:9 to 1:6). The nonpolar fraction (16 mg after evaporation) consisted predominantly of the originally expected product, 6-(4-fluorophenyl)-3,4-dihydro-1-methoxy-1H-benzo[c][1,2]thiazine-2,2-dioxide, of low purity. ¹H NMR (CDCl₃, 400 MHz) δ 7.49, 7.12 (AA′XX′ multiplet with additional H—F coupling, 4H), 7.44 (dd, 1H, J=8.5, 2.1 Hz), 7.34 (d, 1H, J=1.9 Hz), 7.03 (d, 1H, J=8.5 Hz), 3.52, 3.38 (AA′XX′ multiplet, 4H, J_{AX}+J_{AX′}=13.8 Hz), 3.34 (s, 3H).

The main fraction consisted, according to its ¹H NMR spectrum, of two N-demethoxylated compounds, that derived from the coupling product and that derived from the hydrodehalogenation product. This material upon recrystallization from ethanol (reflux to ambient temperature to freezer) yielded 98 mg of crystals in which the ratio of constituents had improved to 8:1. Another recrystallization from ethanol (same temperature interval) finally gave 78 mg (25%) of the title compound in the form of yellowish, compact crystals. ¹H NMR (DMSO-d₆, 400 MHz) δ 10.23 (br s, 1H), 7.65, 7.26 (AA′XX′ multiplet with additional H—F coupling, 4H), 7.51 (d, 1H, J=2.0 Hz), 7.46 (dd, 1H, J=8.3, 2.1 Hz), 6.83 (d, 1H, J=8.3 Hz), 3.38 (s, 4H).

### 1-[[5-[5-(Difluoromethyl)-1,3,4-oxadiazol-2-yl)pyridin-2-yl]methyl]-6-(4-fluorophenyl)-3,4-dihydro-1H-benzo[c][1,2]thiazine-2,2-dioxide

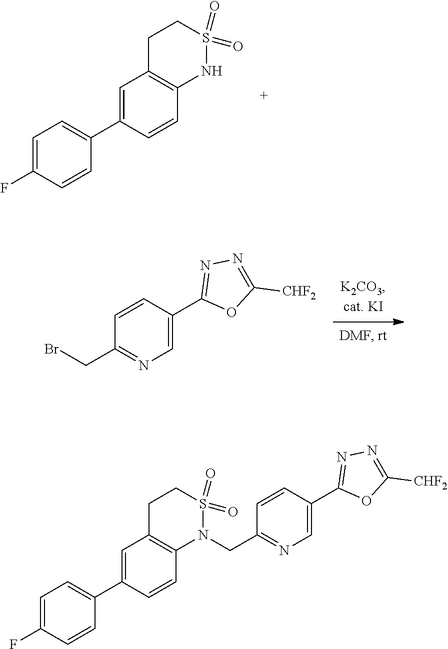 [SMILES: O=S1(=O)CCc2cc(-c3ccc(F)cc3)ccc2N1 ; FC(F)c1nnc(-c2ccc(CBr)nc2)o1 ; O=S1(=O)CCc2cc(-c3ccc(F)cc3)ccc2N1Cc1ccc(-c2nnc(C(F)F)o2)cn1]

In a 20 mL vial with stir bar were placed 6-(4-fluorophenyl)-3,4-dihydro-1H-benzo[c][1,2]thiazine-2,2-dioxide (76.5 mg, 276 μmol), 2-[6-(bromomethyl)pyridin-3-yl]-5-(difluoromethyl)-1,3,4-oxadiazole (88 mg, 303 μmol, 1.1 equiv.), finely ground K₂CO₃ (48 mg, 0.35 mmol, 1.25 equiv.), KI (9 mg, 54 μmol, 0.2 equiv.), and anhydrous DMF (0.8 mL). The vial was wrapped in Al foil for light protection, and the mixture was stirred at ambient temperature for 16.5 h. Water (25 mL) and EtOAc/hexane 1:1 (20 mL) were added, and the phases were separated. The aqueous phase was extracted with EtOAc/hexane 1:1 (10 mL), and the combined organic phases were washed with brine (10 mL). TLC (SiO₂, EtOAc/hexane 2:3) showed the product at Rr approx. 0.2. The solution was adsorbed on SiO₂ (1 g) and the material chromatographed on SiO₂ (17×3 cm, EtOAc/hexane 2:3, then 1:1). Evaporation of the product-containing fractions and drying under vacuum yielded the product (125 mg, 93%) as an off-white solid. ¹H NMR (CDCl₃, 400 MHz) δ 9.32 (narrow m, 1H), 8.39 (dd, 1H, J=8.2, 2.2 Hz), 7.76 (d, 1H, J=8.2 Hz), 7.44, 7.10 (AA′XX′ multiplet with additional H—F coupling, 4H), 7.36 (narrow m, 1H), 7.30 (dd, 1H, J=8.5, 1.9 Hz), 6.93 (t, 1H, J_{H-F}=51.6 Hz), 6.87 (d, 1H, J=8.5 Hz), 5.26 (s, 2H), 3.63, 3.56 (AA′BB′ multiplet, 4H).

### Compound 15. 1-[[5-[5-(Difluoromethyl)-1,3,4-oxadiazol-2-yl)pyrimidin-2-yl]methyl]-3,4-dihydro-1H-benzo[c][1,2]thiazine-2,2-dioxide

### 1-(tert-Butoxycarbonyl)-2-(2-methylpyrimidine-5-carbonyl)hydrazine

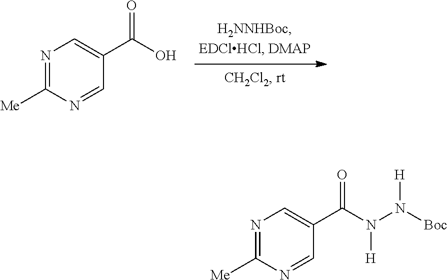 [SMILES: Cc1ncc(C(=O)O)cn1 ; Cc1ncc(C(=O)NNC(=O)OC(C)(C)C)cn1]

2-Methylpyrimidine-5-carboxylic acid (1.93 g, 14.0 mmol) and tert-butylcarbazate (2.22 g, 16.8 mmol) were dissolved in CH₂Cl₂ (50 mL). DMAP (2.22 g, 18.2 mmol, 1.3 equiv.) and EDCI•HCl (3.09 g, 16.1 g, 1.15 equiv.) were added. The flask was fitted with a balloon for exclusion of moisture, and the amber solution was stirred magnetically at rt for 70.5 h. TLC (SiO₂, EtOAc/CH₃CN 3:1) showed, in addition to baseline material, a single mobile, UV-absorbing product (R_{f} approx. 0.45). The reaction mixture was adsorbed on SiO₂ (25 g) and the residue filtered over SiO₂ (9 cm L×6 cm D, EtOAc/CH₃CN 3:1; Note 1). The eluate was evaporated and the residue dried under vacuum to obtain a colorless solid (3.15 g, 89%). ¹H NMR (CDCl₃, 400 MHz) δ 9.02 (s, 2H), 8.42 (br, 1H), 6.77 (br, 1H), 2.81 (s, 3H), 1.50 (s, 9H). A significant impurity displayed a tert-butyl singlet at δ 1.46.
Note 1. Because of relatively high solvent viscosity, a column of good permeability needs to be employed to achieve a practical flow rate. The use of a re-purposed dropping funnel led in the present case to an elution that spread over several days. A courser grade of silica gel than the usual flash chromatography grade may be advantageous.
Note 2. Reaction of the methyl ester with hydrazine hydrate in the same manner as described in the pyridine series resulted in a mixture of products.

### N-(2-Methylpyrimidine-5-carbonyl)hydrazine

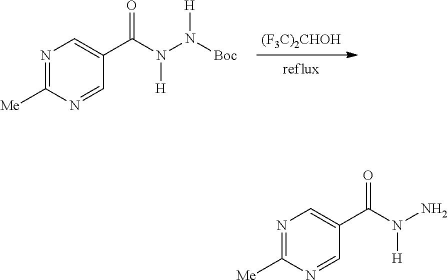 [SMILES: Cc1ncc(C(=O)NNC(=O)OC(C)(C)C)cn1 ; "(F3C)2CHOH" ; Cc1ncc(C(=O)NN)cn1]

A solution of 1-(tert-butoxycarbonyl)-2-(2-methylpyrimidine-5-carbonyl)hydrazine (3.15 g, 12.5 mmol) in 1,1,1,3,3,3-hexafluoro-2-propanol (34 mL) was heated to reflux for 38.5 h in a 250 mL round-bottom flask with stir bar, heating mantle, reflux condenser, and balloon (for exclusion of moisture). Most of the solvent was distilled off at atmospheric pressure. The residue began to solidify on cooling. EtOAc (30 mL) was added, and the mixture was stirred to break up lumps. Suction filtration, washing with EtOAc (2×5 mL), and drying under vacuum yielded a tan powder (1.26 g). The mother liquor was adsorbed on SiO₂(10 g) and the residue chromatographed on SiO₂ (14×3 cm, MeOH/CH₂Cl₂ 1:5). The eluate was evaporated with addition of toluene (10 mL, to effect complete removal of MeOH) to obtain another 0.34 g of the hydrazide as an off-white solid; total: 1.60 g (84%). ¹H NMR (DMSO-d₆, 400 MHz) δ 10.03 (br s, 1H), 9.02 (s, 2H), 4.59 (br s, 2H), 2.66 (s, 3H).

### 5-[5-(Difluoromethyl)-1,3,4-oxadiazol-2-yl]-2-methylpyrimidine

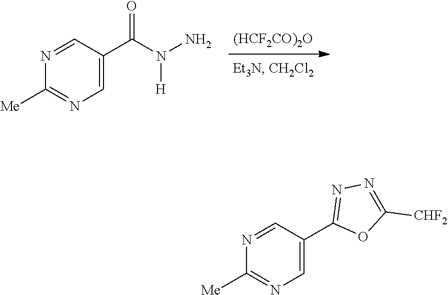

This compound is prepared from N-(2-methylpyrimidine-5-carbonyl)hydrazine in the same manner as described for 5-[5-(difluoromethyl)-1,3,4-oxadiazol-2-yl]-2-methylpyridine.

### 2-(Bromomethyl)-5-[15-(difluoromethyl)-1,3,4-oxadiazol-2-yl]pyridine

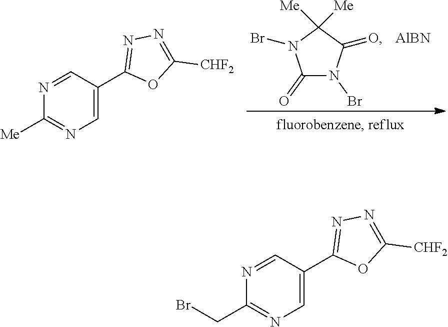

This compound is prepared from 5-[5-(difluoromethyl)-1,3,4-oxadiazol-2-yl]-2-methylpyrimidine in the same manner as described for 2-(bromomethyl)-5-[5-(difluoromethyl)-1,3,4-oxadiazol-2-yl]pyridine.

### 1-[[5-[5-(Difluoromethyl)-1,3,4-axadiazol-2-yl)pyrimidin-2-yl]methyl]-3,4-dihydro-1H-benzo[c][1,2]thiazine-2,2-dioxide

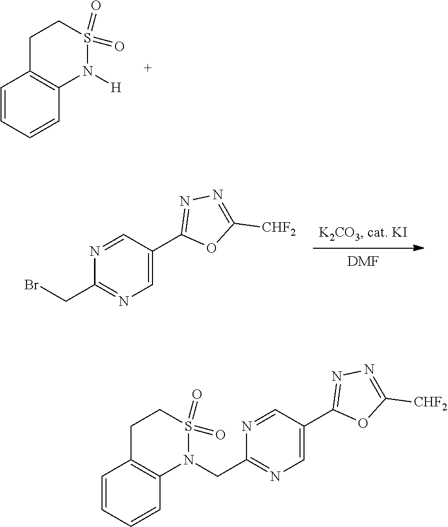

This compound is prepared from 3,4-dihydro-1H-benzo[c][1,2]thiazine-2,2-dioxide and 2-(bromomethyl)-5-[5-(difluoromethyl)-1,3,4-oxadiazol-2-yl]pyrimidine in the same manner as described for 1-[[5-[5-(difluoromethyl)-1,3,4-oxadiazol-2-yl)pyridin-2-yl]methyl]-3,4-dihydro-1H-benzo[c][1,2]thiazine-2,2-dioxide.

### Compound 31. 1-[[5-[5-(Trifluoromethyl)-1,2,4-oxadiazol-3-yl]pyridin-2-yl]methyl]-3,4-dihydro-1H-benzo[c][1,2]thiazine-2,2-dioxide

### 2-Methyl-5-[5-(trifluoromethyl)-1,2,4-oxadiazol-3-yl]pyridine

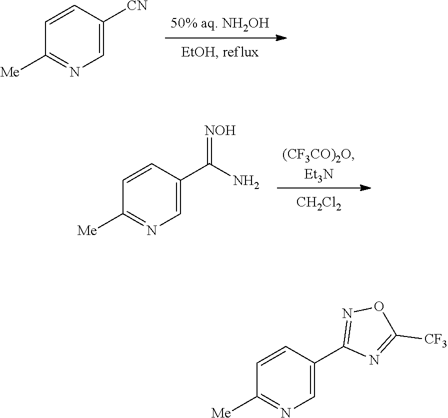

In a 150 mL round-bottom flask with stir bar and reflux condenser (open to the atmosphere) were placed 6-methylnicotinonitrile (2.23 g, 18.9 mmol), EtOH (non-denatured, 20 mL), and 50% aqueous hydroxylamine solution (15.1 M, 1.50 mL, 22.7 mmol, 1.2 equiv.). The mixture was heated to reflux for 23 h. After cooling, TLC (SiO₂, MeOH/CHCl₃ 1:9) showed one major spot at R_{f} approx. 0.25. Toluene (20 mL) was added, and the mixture was evaporated to dryness and the residue dried under vacuum. The crude amidoxime formed an off-white powder and was carried on without purification.

The amidoxime was suspended in CH₂Cl₂ (180 mL) and transferred into a 500 mL round-bottom flask equipped with stir bar, dropping funnel, and balloon for exclusion of moisture. With ice cooling, trifluoroacetic anhydride (11.9 mL, 84.3 mmol, 4.45 equiv.) was added dropwise in 20 min to obtain a clear solution. The cold bath was removed and the mixture stirred for 2 h. Triethylamine (19.5 mL, 140 mmol, 7.4 equiv.) was added dropwise in 10 min at ambient temperature, resulting in a mild exotherm. The mixture was stirred at ambient temperature for 3 h. Shortly before this time point, TLC (SiO₂, EtOAc/hexane 1:4) showed the desired product at R_{f} approx. 0.4, with major byproducts at R_{f} approx. 0.7 and 0.15 and several minor byproducts. Evaporation gave an amber syrup, which was taken up in EtOAc and adsorbed on SiO₂ (50 g). Chromatography of the residue on SiO₂ (22×5 cm, EtOAc/hexane 1:5) yielded, after a deep-orange forerun, still impure product-containing fractions, which upon evaporation left 3.3 g of a dark orange-red oil together with some solid. TLC analysis of this material (SiO₂, EtOAc/toluene 1:4) showed the desired product as the major component at R_{f} approx. 0.25, accompanied by multiple nonpolar impurities and some baseline material; in particular, one of these byproducts closely preceded the oxadiazole. The oil was applied on a SiO₂ column (30×5 cm), which was eluted with EtOAc/toluene 1:6. Product-containing fractions (still contaminated with some of the closely preceding spot) were evaporated to leave 2.04 g of an oil. This material was finally purified by bulb-to-bulb distillation (approx. 60-65° C./oil pump) to obtain 1.62 g (37%) of the oxadiazole as a yellowish, mobile oil (a substantial amount of amber, crystalline residue was observed). ¹H NMR (400 MHz, CDCl₆) δ 9.23 (narrow m, 1H), 8.27 (dd, 1H, J=8.1, 2.3 Hz), 7.34 (d, 1H, J=8.1 Hz), 2.67 (s, 3H). ¹³C NMR (100 MHz, CDCl₆) δ 167.45, 166.05 (q, J_{C-F}=44.6 Hz), 162.76, 148.21, 135.09, 123.48, 118.51, 115.88 (q, J_{C-F}=273.9 Hz), 24.72.

### 2-(Bromomethyl)-5-[5-(trifluoromethyl)-1,2,4-oxadiazol-3-yl]pyridine

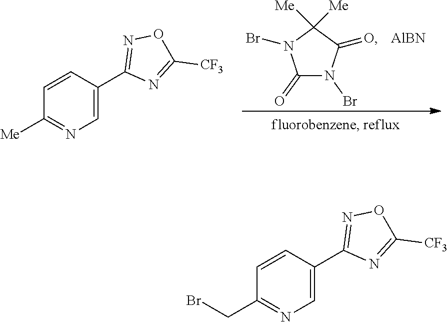

In a 100 mL 3-necked flask with stir bar, heating mantle, stopper, reflux condenser (connected to an Ar balloon), and dropping funnel with septum were placed 2-methyl-5-[5-(trifluoromethyl)-1,2,4-oxadiazol-3-yl]pyridine (1.60 g, 6.98 mmol), 1,3-dibromo-5.5-dimethylhydantoin (1.10 g, 3.84 mmol, 0.55 equiv.), and fluorobenzene (12 mL). The atmosphere was replaced with Ar. The dropping funnel was charged with a solution of azobis(isobutyronitrile) (AIBN; 0.17 g, 1.05 mmol, 0.15 equiv.) in fluorobenzene (2 mL), of which approx. 10% was added to the reaction mixture. The mixture was heated to reflux, and the remainder of the AIBN solution was added in small portions over the course of 1.5 h. At this point, most of the color of the temporarily formed Br₂ had faded. Reflux was continued for 10 min, and the mixture was cooled to ambient temperature. TLC (SiO₂, EtOAc/hexane 1:4 or 1:9) showed, besides baseline material, three major components at R_{f} approx. 0.8, 0.6, and 0.35 or at R_{f} approx. 0.6, 0.35, and 0.2, respectively, which corresponded to the dibromination byproduct, the desired monobromination product, and starting material. The dark solution was adsorbed on SiO₂ (8 g), and the residue was chromatographed on SiO₂ (27×4 cm, EtOAc/hexane 1:12). Evaporation of the monobromide-containing fractions yielded 0.83 g (38%) of a colorless, crystalline solid of good purity. ¹H NMR (400 MHz, CDCl₆) δ 9.30 (narrow m, 1H), 8.41 (dd, 1H, J=8.1, 2.1 Hz), 7.64 (d, 1H, J=8.2 Hz), 4.62 (s, 2H).

### 1-[[5-[5-(Trifluoromethyl)-1,2,4-oxadiazol-3-yl]pyridin-2-yl]methyl]-3,4-dihydro-1H-benzo[c][1,2]thiazine-2,2-dioxide

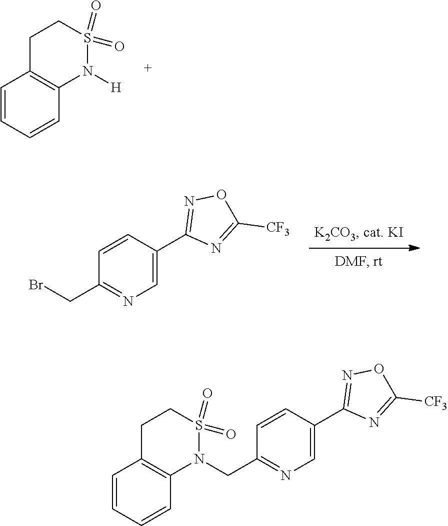 [SMILES: O=S1(=O)CCc2ccccc2N1 ; FC(F)(F)c1nc(-c2ccc(CBr)nc2)no1 ; "DMF, rt" ; O=S1(=O)CCc2ccccc2N1Cc1ccc(-c2noc(C(F)(F)F)n2)cn1]

In a 100 mL round-bottom flask with stir bar were placed 3,4-dihydro-1H-benzo[c][1,2]thiazine-2,2-dioxide (181 mg, 988 μmol), 3-[6-(bromomethyl)pyridin-3-yl]-5-(trifluoromethyl)-1,2,4-oxadiazole (320 mg, 1.04 mmol, 1.05 equiv.), finely ground K₂CO₃ (165 mg, 1.20 mmol, 1.2 equiv.), KI (33 mg, 0.20 mmol, 0.20 equiv.), and anhydrous DMF (3.0 mL). The flask was wrapped in Al foil for light protection, and the mixture was stirred at ambient temperature for 25.5 h. Water (50 mL) and EtOAc/hexane 1:1 (50 mL) were added, and the phases were separated. The organic phase was washed with water (50 mL). TLC (SiO₂, EtOAc/hexane 3:7) showed the product at R_{f} approx. 0.3, indistinguishable from the starting sultam, while the alkylating agent was absent (reference spot, R_{f} approx. 0.7). With EtOAc/CHCl₃ 1:9, the product was detected at R_{f} approx. 0.5, while the starting sultam was absent (reference spot, R_{f} approx. 0.4). The solution was adsorbed on SiO₂ (4 g) and the material chromatographed on SiO₂ (16×3 cm, EtOAc/hexane 3:7; tailing). Evaporation of the product-containing fractions and drying under vacuum yielded the product (392 mg, 97%) as a very pale yellowish glass, which gradually solidified on standing. ¹H NMR (CDCl₃, 500 MHz) δ 9.29 (narrow m, 1H), 8.36 (dd, 1H, J=8.2, 2.1 Hz), 7.71 (d, 1H, J=8.2 Hz), 7.19 (d, 1H, J=7.5 Hz), 7.13 (t, 1H, J=7.7 Hz), 7.02 (t, 1H, J=7.6 Hz), 6.82 (d, 1H, J=8.2 Hz), 5.22 (s, 2H), 3.56, 3.50 (AA′BB′ multiplet, 4H); ¹³C NMR (CDCl₃, 125 MHz) δ 167.13, 166.21 (q, J_{C-F}=44.8 Hz), 161.36, 148.39, 139.85, 136.13, 129.72, 127.90, 123.38, 122.28, 121.73, 120.17, 117.78, 115.82 (q, J_{C-F}=273.9 Hz), 51.93, 45.78, 28.22.

### Example 2: Exemplary In Vitro Activity of Compounds Disclosed Herein

### Hdac Enzyme Activity Inhibition Assay

The effectiveness, or potency, of a present HDAC6I with respect to inhibiting the activity of an HDAC is measured by an IC₅₀ value. The quantitative IC₅₀ value indicates the concentration of a particular compound that is needed to inhibit the activity of an enzyme by 50% in vitro. Stated alternatively, the IC₅₀ value is the half maximal (50%) inhibitory concentration of a compound tested using a specific enzyme, e.g., HDAC, of interest. The smaller the IC₅₀ value, the more potent the inhibiting action of the compound because a lower concentration of the compound is needed to inhibit enzyme activity by 50%.

In preferred embodiments, a present HDAC6I inhibits HDAC enzymatic activity by about at least 50%, preferably at least about 75%, at least 90%, at least 95%, or at least 990.

Compounds of the present invention were tested for IC₅₀ values against both HDAC6 and HDAC1. In some embodiments, a present compound also was tested against HDAC1, 2, 3, 4, 5, 8, 10, and 11. The tested compounds showed a range of IC₅₀ values vs. HDAC6 of about 1 nM to greater than 30 μM, and a range of IC₅₀ values vs. HDAC1 of about 91 nM to greater than 30 μM. Therefore, in some embodiments, a present HDAC6I is a selective HDAC6 inhibitor which, because of a low affinity for other HDAC isozymes, e.g., HDAC1, gives rise to fewer side effects than compounds that are non-selective HDAC inhibitors.

In some embodiments, the present HDACIs interact with and reduce the activity of all histone deacetylases in a cell. In some preferred embodiments, the present HDACIs interact with and reduce the activity of fewer than all histone deacetylases in the cell. In certain preferred embodiments, the present HDACIs interact with and reduce the activity of one histone deacetylase (e.g., HDAC6), but do not substantially interact with or reduce the activities of other histone deacetylases (e.g., HDAC1, HDAC2, HDAC3, HDAC4, HDAC5, HDAC7, HDAC8, HDAC9, HDAC10, and HDAC11). The present invention therefore provides HDAC6Is for the treatment of a variety of diseases and conditions wherein inhibition of HDAC6 has a beneficial effect. Preferably, a present HDAC6I is selective for HDAC6 over the other HDAC isozymes by a factor of at least 2, at least 5, at least 10, at least 20, at least 50, at least 100, at least 500, at least 1000, at least 2000, at least 3000, and preferably up to about 4000. For example, in various embodiments, a present HDAC6I exhibits an IC₅₀ value versus HDAC6 that is about 350 or about 1000 times less than the IC₅₀ value vs. HDAC1, i.e., a selectivity ratio (HDAC1 IC₅₀/HDAC6 IC₅₀) of about 350 or about 1000.

Other assays also showed a selectivity of a present HDAC6I for HDAC6 over HDAC1, 2, 3, 4, 5, 8, 10, and 11 of about 1000.

The IC₅₀ values for compounds of structural formula (I) vs. HDACI and HDAC6 were determined as follows:

The HDAC1, 2, 4, 5, 6, 7, 8, 9, 10, and 11 assays used isolated recombinant human protein; HDAC3/NcoR2 complex was used for the HDAC3 assay. Substrate for HDAC1, 2, 3, 6, 10, and 11 assays is a fluorogenic peptide from p53 residues 379-382 (RHKKAc); substrate for HDAC8 is fluorogenic diacyl peptide based on residues 379-382 of p53 (RHKAcKAc). Acetyl-Lys(trifluoroacetyl)-AMC substrate was used for HDAC4, 5, 7, and 9 assays. Compounds were dissolved in DMSO and tested in 10-dose IC₅₀ mode with 3-fold serial dilution starting at 30 μM. Control compound trichostatin A (TSA) was tested in a 10-dose IC₅₀ mode with 3-fold serial dilution starting at 5 μM. IC₅₀ values were extracted by curve-fitting the dose/response slopes. Assays were performed in duplicate, and IC₅₀ values are an average of data from both experiments.

### Materials

Human HDAC1 (GenBank Accession No. NM_004964): Full length with C-terminal GST tag, MW=79.9 kDa, expressed by baculovirus expression system in Sf9 cells. Enzyme is in 50 mM Tris-HCl, pH 8.0, 138 mM NaCl, 20 mM glutathione, and 10% glycerol, and stable for >6 months at −80° C. Purity is >10% by SDS-PAGE. Specific Activity is 20 U/μg, where one U=1 pmol/min under assay condition of 25 mM Tris/Cl, pH 8.0, 137 mM NaCl, 2.7 mM KCl, 1 mM MgCl₂, 0.1 mg/mL BSA, 100 μM HDAC substrate, and 13.2 ng/μL HDAC1, incubation for 30 min at 30° C. Human HDAC6 (GenBank Accession No. BC069243): Full length with N-terminal GST tag, MW=159 kDa, expressed by baculovirus expression system in Sf9 cells. Enzyme is in 50 mM Tris-HCl, pH 8.0, 138 mM NaCl, 20 mM glutathione, and 10% glycerol, and stable for >6 months at −80° C. Purity is >90°/by SDS-PAGE. Specific Activity is 50 U/μg, where one U=1 pmol/min under assay condition of 25 mM Tris/C₁, pH 8.0, 137 mM NaCl, 2.7 mM KCl, 1 mM MgCl₂, and 0.1 mg/mL BSA, 30 μM HDAC substrate, and 5 ng/μL HDAC6, incubation for 60 min at 30° C.

Substrate for HDAC1 and HDAC6: Acetylated peptide substrate for HDAC, based on residues 379-382 of p53 (Arg-His-Lys-Lys(Ac)), a site of regulatory acetylation by the p300 and CBP acetyltransferases (lysines 381, 382)1-6, is the best for HDAC from among a panel of substrates patterned on p53, histone H₃, and histone H₄ acetylation sites7.
References: Gu, W. et al., Cell 1997, 90, 595; Sakaguchi, K. et al., Genes Dev. 1998, 12, 2831; Liu, L. et al., Mal. Cell. Biol. 1999, 19, 1202: Ito, A. et al., EMBO J., 2001, 20, 1331; Barlev, N. A. et al., Mal. Cell 2001, 8, 1243. Ito, A. et al., EMBO J. 2002, 21, 6236.

Reaction Buffer: 50 mM Tris-HCl, pH 8.0, 137 mM NaCl, 2.7 mM KCl, 1 mM MgCl₂, 1 mg/mL BSA.

### Assay Conditions

HDAC1: 75 nM HDAC1 and 50 μM HDAC substrate are in the reaction buffer and 1% DMSO final. Incubate for 2 hours at 30° C.

HDAC6: 12.6 nM HDAC6 and 50 μM HDAC substrate are in the reaction buffer and 1% DMSO final. Incubate for 2 hours at 30° C.

### IC₅₀ Value Calculations

All IC₅₀ values are automatically calculated using the GraphPad Prism version 5 and Equation of Sigmoidal dose-response (variable slope):

Y=Bottom+(Top-Bottom)/(I+10″((Log EC₅₀−X)*HillSlope)), where X is the logarithm of concentration, Y is the response, and Y starts at Bottom and goes to Top with a sigmoid shape. In most cases, “Bottom” is set 0, and “Top” is set “less than 120%”. This is identical to the “four parameter logistic equation”. IC₅₀ curves also are drawn using the GraphPad Prism, and IC₅₀ values and Hill slopes are provided.

HDAC Activity Assays: HDAC assay is performed using fluorescently-labeled acetylated substrate, which comprises an acetylated lysine side chain. After incubation with HDAC, deacetylation of the substrate sensitizes the substrate such that, in a second step, treatment with the detection enzyme produces a fluorophore. HDACs 1 and 6 were expressed as full length fusion proteins. Purified proteins were incubated with 50 μM fluorescently-labeled acetylated peptide substrate and test compound for 2 hours at RT in HDAC assay buffer containing 50 mM Tris-HCl (pH 8.0), 137 mM NaCl, 2.7 mM KCl, 1 mM MgCl₂, 1% DMSO, and 1% BSA.

Reactions were terminated by the addition of the developer after 2 hours, and the development of fluorescence signal, which was relative to the amount of deacetylated peptide, was monitored by time-course measurement of EnVision (PerkinElmer). The HDAC activity was estimated from the slope of time-course measurement of the fluorescence intensity. The slope of no-enzyme control (substrate alone) was used as background, and % Enzyme activity was calculated using background-subtracted slope of no inhibitor control (DMSO) as 100% activity.

To date, HDACIs have demonstrated a relatively non-specific inhibition of various HDAC isozymes. Most HDACIs so far identified primarily inhibit HDAC 1, 2, 3, and 8, producing an anti-proliferative phenotype which is useful for oncology applications, but not for the many non-oncology applications of HDAC6Is. (K. B. Glaser et al., Biochem. Biophys. Res. Commun. 2003, 310, 529-536.) The potential toxicities associated with the inhibition of certain HDAC isozymes can lead to additional difficulties for the clinical development of pan-HDAC, i.e., nonselective HDAC, inhibitors. Because the network of cellular effects mediated by acetylation is so vast and because inhibition of some HDAC isozymes may lead to undesirable side effects, HDAC isozyme selective inhibitors hold a greater therapeutic promise than their nonselective counterparts.

Several pan-selective compounds have been approved by the FDA, although for use in cutaneous T-cell lymphoma and multiple myeloma (Kelly, W. K. et al., Nat. Clin. Pract. Oncol. 2005, 2, 150-157). Avoiding cytotoxicity through isozyme selectivity would ultimately prove advantageous and may open doors to a variety of other therapeutic areas.

### Cells

The human melanoma cell line WM164 was obtained from Dr. Smalley's Lab at Moffitt Cancer Center. Cells were cultured in RPMI 1640 media, supplemented with 10% FBS, penicillin/streptomycin (50 U/mL), L-glutamine (2 mM), and 2-mercaptoethanol (50 mM) (complete media), and grown under humidified conditions at 37° C. and 5% CO₂.

### Assay Conditions

WM164 melanoma cells were plated at 105 cells/well in 12-well plates and allowed to adhere overnight. A 50 mM stock of compound was then added by serial dilutions in complete medium to the indicated concentrations. Cells were incubated for 24 h under humidified conditions (37° C., 5% CO₂). Wells were then washed with cold PBS, and cells were lysed in a buffer containing 10 mM Tris-HCl pH 8.0, 10% SDS, 4 mM urea, 100 mM DTT, and 1× protease inhibitor (Roche). Cells were lysed for 30 min on ice and then sonicated for 8 min (8 cycles of 30 s on/30 s rest). Cells were then boiled for 10 min with 6× gel loading buffer and resolved on 4-15% gradient gels and subsequently transferred onto nitrocellulose membranes. Membranes were blocked with 5% milk in PBS-T, and specific antigens were detected using antibodies against acetyl-H3 and H3 (Cell Signaling) and acetyl-α-tubulin and α-tubulin (Sigma). Bands were detected by scanning blots with an LI-COR Odyssey imaging system using both 700 and 800 channels.

The hyperacetylation of α-tubulin without elevating levels of acetylated histones is a hallmark of HDAC6 inhibition. HDAC6 contains two catalytic domains. Its C-terminal domain is the functional domain for both synthetic and physiological substrates, whereas the N-terminal domain is devoid of enzymatic activity (Zou, H. et al., Biochem. Biophys. Res. Commun., 2006, 341, 45-50). To assess the activity of the compounds to work in cells, the ability of some of the HDAC inhibitors to induce increased levels of tubulin acetylation was assessed. The western blots are shown in FIG. 1. Low micromolar treatment of example compounds on WM 164 melanoma cells led to a dose-dependent increase of acetyl α-tubulin levels without a concomitant elevation of histone H3 acetylation (FIG. 1) indicating binding to the second, enzymatically active catalytic domain. Not until concentrations of 1 and 10 μM were used was an observable increase in histone H3 acetylation found. This is not surprising as the biochemical IC₅₀ of example compounds against the class 1 HDACs, those responsible for histone acetylation, is in the micromolar range. There is a clear preference for activity in a cellular environment that corresponds to selective HDAC6 inhibition.

### Cytotoxicity Vs HDAC Inhibitory Activity

### Cells

The B16-F10-luc murine melanoma cell line was obtained from ATCC and cultured in RPMI 1640 supplemented with 10% FBS, 100 IU/mL penicillin, and 100 mg/mL streptomycin. The SM1 cell line was obtained from Dr. Antoni Ribas's lab at University of California, Los Angeles. Human melanoma cell line WM164 was obtained from Dr. Smalley's Lab at Moffitt Cancer Center. Cells were cultured in RPMI 1640 medium, supplemented with 10% FBS, penicillin/streptomycin (50 U/mL), L-glutamine (2 mM), and 2-mercaptoethanol (50 mM) (complete media), and grown under humidified conditions at 37° C. and 5% CO₂.

### Assay Conditions

Murine melanoma cells were plated at 5×10³/well in 96-well flat-bottom plates. The following day, medium was changed to that containing various concentrations of HDAC6I or matched DMSO vehicle concentrations diluted in complete medium performed in triplicate. Cells were incubated for 48 h at 37° C. and 5% CO₂. The density of viable, metabolically active cells was quantified using a standard MTS assay (CellTiter 96 AQueous One, Promega, Madison, WI) as per manufacturer's instructions. Briefly, 20 μL of reagent was added per well and incubated at 37° C. for 3 h. Absorbances at 490 nM were measured spectrophotometrically with background subtraction at 690 nM. All values were then normalized and expressed as a percentage of medium control (100%).

### Analysis of Compound 1

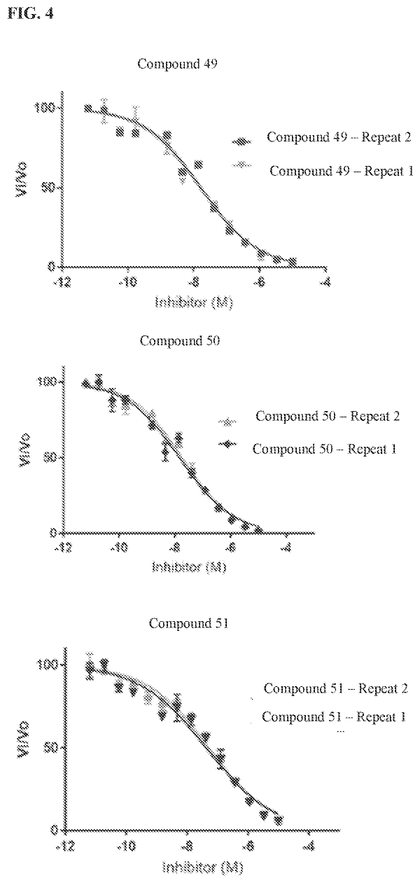

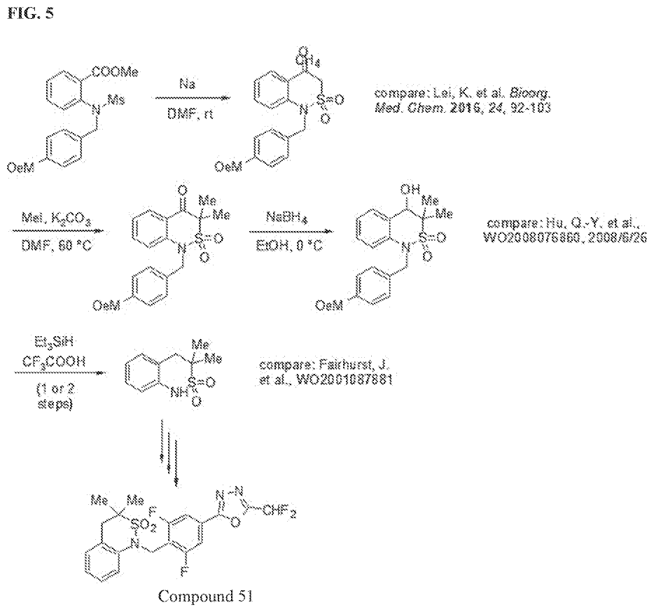

Compound 1 displays 29 nM potency at hHDAC6 (duplicate) as performed by DRC and shows full selectivity against HDAC1 (DRC), but also versus other HDAC subtypes (% inh. at 10 μM: <20%, see below and raw data) (FIG. 3A).

Compound 1 has high kinetic solubility, high permeability, and low efflux. It is characterized by a low clearance in human microsomes, but higher clearance in mouse microsomes. The free fractions in both mouse and human plasma are high. The compound is chemically stable at pH 7.4 and 2. Though the Clint being higher that the set criteria in Schedule B of 50 ul/min/mg protein, we agree to pursue the profiling in mice PK studies in order to inform on the iv-IV Clearance relationship. (FIG. 3B).

The results of the mice iv PK of Compound 1 indicate a clearance of 44.9 mL/min/kg is observed, which is about half of the mouse hepatic blood flow. This medium clearance is in line with the observed microsomal stability. The oral PK study of Compound 1 indicate a medium clearance, medium volume of distribution, half-life 2.2. h and mean residence time 3.14 h, and a lower oral bioavailability (14.7%). The mean Cmax value of 456 ng/mL is noted rather shortly after the gavage (Tmax=15 min). The compound has been detected in the brain and the sciatic nerve with moderate concentrations with a Brain/Plasma ratio of 0.8 (@1 h) to 1.2 (@4 h) and Sciatic nerve/plasma ratio of 0.4 at 1 h.

The PK/BBB properties of Compound 1 in combination with the reported functional activity (0.76 uM) might not be sufficiently good yet in order to consider this compound for further in vivo evaluation (at least not in mice).

Compound 1 may be a promising starting point to optimize the compounds towards more potent, selective and brain penetrant HDAC6 inhibitors for CNS applications. Additional characterization of Compound 1 in other species as well as optimization of functional biological activity and ADME-T/PK properties of future molecules remains to be done, followed by appropriate efficacy studies that will be required toward the identification of preclinical candidates for CNS applications.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound having General Formula I: 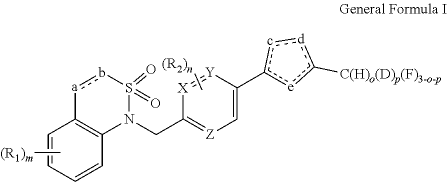
or a pharmaceutically acceptable salt and/or solvate thereof, wherein: [symbol]
represents a single or double bond; in the case of the five-membered ring moiety, there are two double bonds and three single bonds, together arranged in such manner as to give rise to a five-membered ring heteroaryl group;
X and Y are independently carbon or nitrogen, and Z is N or C(F);
m=0, 1, or 2; except when R₁ is deuterium, m is an integer ranging from 0 to 4;
n=0, 1, or 2 if at least one of X and Y is carbon; or n=0 or 1 if both X and Y are nitrogen;
the sum of o and p is 0 or 1;
R₁ and R₂ are independently selected from the group consisting of hydrogen, deuterium, halogen, hydroxyl, —CH₂OH, —CH₂CH₂OH, cyano, —NRₐR_{b}, —CH₂NRₐR_{b}, —C(O)NRₐR_{b}, —S(O)₂NRₐR_{b}, acetyl, C₁-C₆ alkyl, C₁-C₆ alkenyl, C₁-C₆ alkynyl, C₁-C₆ alkoxy, C₁-C₆ haloalkyl, F₃C—S—, C₃-C₆ cycloalkyl, (C₃-C₅ cycloalkyl)-(C₁-C₃alkyl)-, (C₁-C₃alkyl)-(C₃-C₅ cycloalkyl)-, (C₃-C₅ cycloalkyl)-O—, aryl, aryl-O—, aryl-(C₁-C₃ alkyl)-, aryl-(C₁-C₃ alkoxy)-, heteroaryl, heteroaryl-O—, heteroaryl-(C₁-C₃ alkyl)-, heteroaryl-(C₁-C₃ alkoxy)-, 4- to 6-membered heterocyclyl, or (4- to 6-membered heterocyclyl)-(C₁-C₃ alkyl)-; or two of R₁ and/or two of R₂ are attached to adjacent carbon atoms of their respective aromatic rings and are connected to form a five- or six-membered carbo- or heterocyclic ring;
Rₐ and R_{b} are independently selected from the group consisting of hydrogen and C₁-C₆ alkyl or C₃-C₆ cycloalkyl, or these groups may be joined to form a 3- to 7-membered heterocyclyl;
a is CR_{c}R_{d}, C═O, Nₑ, O, or S when [symbol] represents a single bond, or CR_{c} or N when [symbol] represents a double bond;
b is CR_{c}R_{d} when [symbol] represents a single bond or CR_{c} when [symbol] represents a double bond, or b is N when [symbol] represents a double bond and a is CR_{c};
R_{c} and R_{d} are independently hydrogen, deuterium, fluorine, chlorine, C₁-C₆ alkyl, or C₃-C₆ cycloalkyl, or they are joined together to form a 3- to 6-membered cycloalkyl;
Rₑ is selected from the group consisting of hydrogen, C₁-C₆ alkyl, C₃-C₆ cycloalkyl, aryl, heteroaryl, or 4- to 6-membered heterocyclyl;
one of c, d, and e is oxygen, and the remainder are nitrogen; or c is C(R_{f}), one of d and e is oxygen, and the other is nitrogen; and
R_{f} is hydrogen, deuterium, fluorine, chlorine, methyl, difluoromethyl, trifluoromethyl, or cyano.

2. A compound according to claim 1 of formula Ib 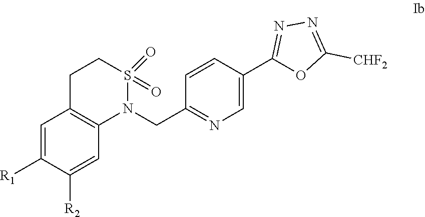
wherein
R₁ and R₂ are independently selected from the group consisting of hydrogen, deuterium, halogen, hydroxyl, —CH₂OH, —CH₂CH₂OH, cyano, —NRₐR_{b}, —CH₂NRₐR_{b}, —C(O)NRₐR_{b}, —S(O)₂NRₐR_{b}, acetyl, C₁-C₆ alkyl, C₁-C₆ alkenyl, C₁-C₆ alkynyl, C₁-C₆ alkoxy, C₁-C₆ haloalkyl, F₃C—S—, C₃-C₆ cycloalkyl, (C₃-C₅ cycloalkyl)-(C₁-C₃ alkyl)-, (C₁-C₃ alkyl)-(C₃-C₅ cycloalkyl)-, (C₃-C₅ cycloalkyl)-O—, aryl, aryl-O—, aryl-(C₁-C₃ alkyl)-, aryl-(C₁-C₃ alkoxy)-, heteroaryl, heteroaryl-O—, heteroaryl-(C₁-C₃ alkyl)-, heteroaryl-(C₁-C₃ alkoxy)-, 4- to 6-membered heterocyclyl, or (4- to 6-membered heterocyclyl)-(C₁-C₃ alkyl)-;
Rₐ and R_{b} are independently selected from the group consisting of hydrogen and C₁-C₆ alkyl or C₃-C₆ cycloalkyl, or these groups may be joined to form a 3- to 7-membered heterocyclyl;

3. A compound according to claim 1 of formula Ib, wherein R₁ and R₂ are independently selected from H, D, Cl, and F.

4. A compound according to claim 1 of formula Ic 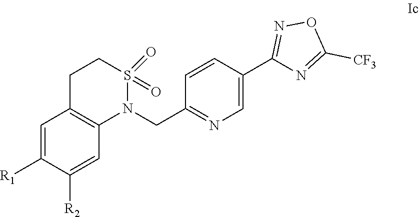
wherein R₁ and R₂ are defined as in claim 1.

5. A compound according to claim 1 of formula Ic, wherein R₁ and R₂ are independently selected from H, D, Cl, and F.

6. A compound according to claim 1 of formula Id 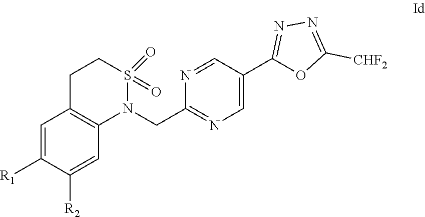
wherein R₁ and R₂ are defined as in claim 1.

7. A compound according to claim 1 of formula Id, wherein R₁ and R₂ are independently selected from H, D, Cl, and F.

8. A compound according to claim 1 of formula Ie 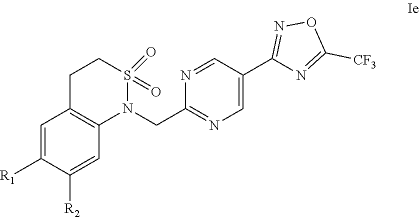
wherein R₁ and R₂ are defined as in claim 1.

9. A compound according to claim 1 of formula Ie, wherein R₁ and R₂ are independently selected from H, D, Cl, and F.

10. A compound according to claim 1 of formula If 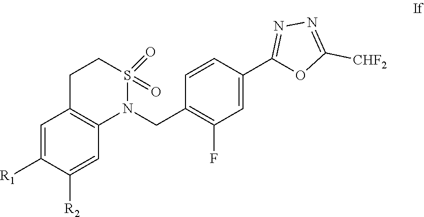
wherein R₁ and R₂ are defined as in claim 1.

11. A compound according to claim 1 of formula If, wherein R₁ and R₂ are independently selected from H, D, Cl, and F.

12. A compound according to claim 1 of formula Ig 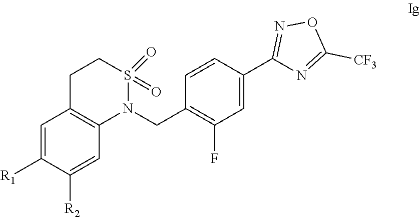
wherein R₁ and R₂ are defined as in claim 1.

13. A compound according to claim 1 of formula Ig, wherein R₁ and R₂ are independently selected from H, D, Cl, and F.

14. A compound according to claim 1 of formula Ih 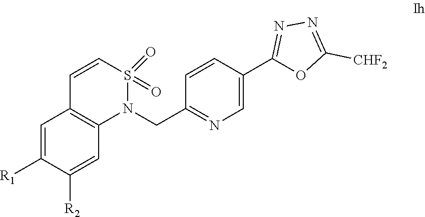
wherein R₁ and R₂ are defined as in claim 1.

15. A compound according to claim 1 of formula Ih, wherein R₁ and R₂ are independently selected from H, D, Cl, and F.

16. A compound according to claim 1 of formula Ii 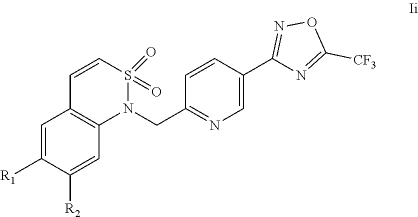
wherein R₁ and R₂ are defined as in claim 1.

17. A compound according to claim 1 of formula Ii, wherein R₁ and R₂ are independently selected from H, D, Cl, and F.

18. A compound according to claim 1 of formula Ij 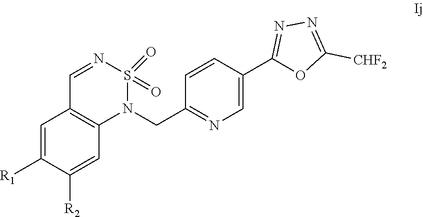
wherein R₁ and R₂ are defined as in claim 1.

19. A compound according to claim 1 of formula Ij, wherein R₁ and R₂ are independently selected from H, D, Cl, and F.

20. A compound according to claim 1 of formula Ik 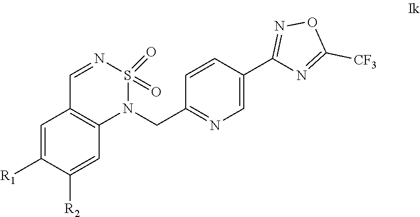
wherein R₁ and R₂ are defined as in claim 1.

21. A compound according to claim 1 of formula Ik, wherein R₁ and R₂ are independently selected from H, D, Cl, and F.

22. A compound according to claim 1 of formula IL 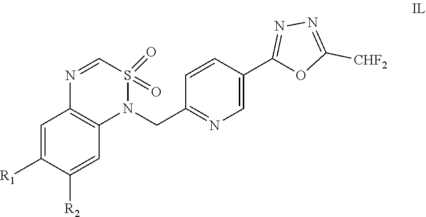
wherein R₁ and R₂ are defined as in claim 1, and R₃ is methyl, or both of R₃ together are CH₂CH₂.

23. A compound according to claim 1 of formula IL, wherein R₁ and R₂ are independently selected from H, D, Cl, and F.

24. A compound according to claim 1 of formula Im 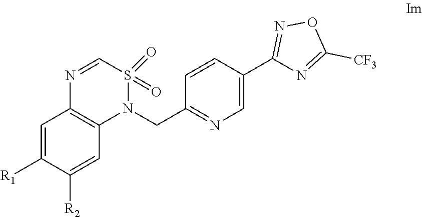
wherein R₁ and R₂ are defined as in claim 1.

25. A compound according to claim 1 of formula Im, wherein R₁ and R₂ are independently selected from H, D, Cl, and F.

26. A compound according to claim 1 of formula In 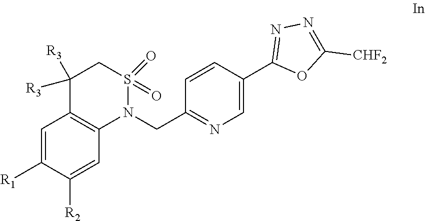
wherein R₁ and R₂ are defined as in claim 1, and both of R₃ are methyl, or one of R₃ is methyl and the other is H, or both of R₃ together are CH₂CH₂ or CH₂CH₂CH₂.

27. A compound according to claim 1 of formula In, wherein R₁ and R₂ are independently selected from H, D, Cl, and F.

28. A compound according to claim 1 of formula Io 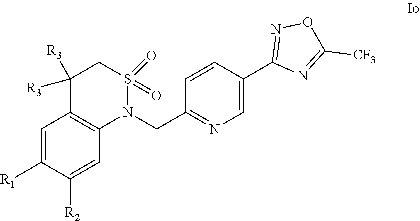
wherein R₁ and R₂ are defined as in claim 1, and both of R₃ are methyl, or one of R₃ is methyl and the other is H, or both of R₃ together are CH₂CH₂ or CH₂CH₂CH₂.

29. A compound according to claim 1 of formula Io, wherein R₁ and R₂ are independently selected from H, D, Cl, and F.

30. A compound according to claim 1 of formula Ip 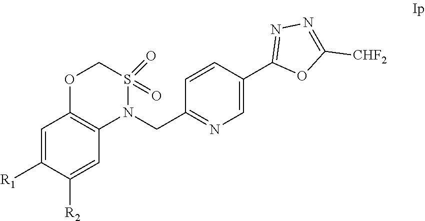
wherein R₁ and R₂ are defined as in claim 1.

31. A compound according to claim 1 of formula Ip, wherein R₁ and R₂ are independently selected from H, D, Cl, and F.

32. A compound according to claim 1 of formula Iq 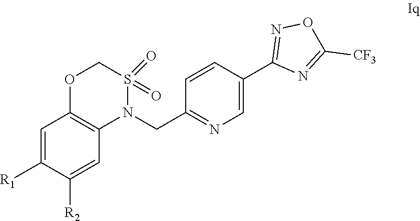
wherein R₁ and R₂ are defined as in claim 1.

33. A compound according to claim 1 of formula Iq, wherein R₁ and R₂ are independently selected from H, D, Cl, and F.

34. A compound according to claim 1 of formula Ir 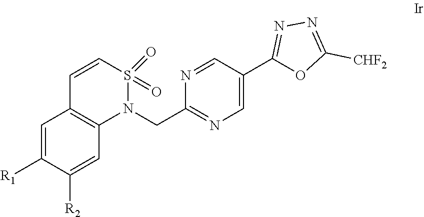
wherein R₁ and R₂ are defined as in claim 1.

35. A compound according to claim 1 of formula Ir, wherein R₁ and R₂ are independently selected from H, D, Cl, and F.

36. A compound according to claim 1 of formula Is 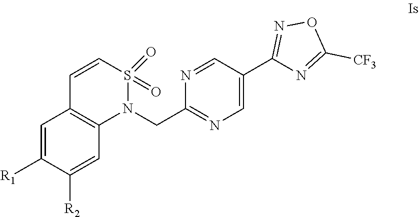
wherein R₁ and R₂ are defined as in claim 1.

37. A compound according to claim 1 of formula Is, wherein R₁ and R₂ are independently selected from H, D, Cl, and F.

38. A compound according to claim 1 of formula It 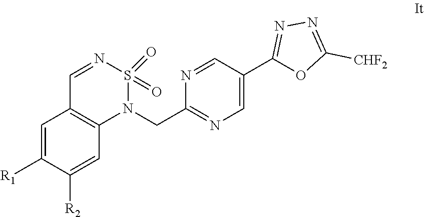
wherein R₁ and R₂ are defined as in claim 1.

39. A compound according to claim 1 of formula It, wherein R₁ and R₂ are independently selected from H, D, Cl, and F.

40. A compound according to claim 1 of formula Iu 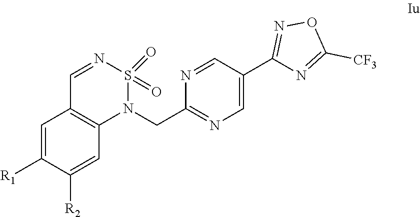
wherein R₁ and R₂ are defined as in claim 1.

41. A compound according to claim 1 of formula Iu, wherein R₁ and R₂ are independently selected from H, D, Cl, and F.

42. A compound according to claim 1 of formula Iv 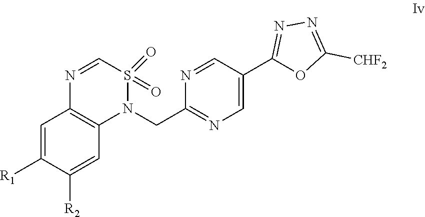
wherein R₁ and R₂ are defined as in claim 1, and R₃ is methyl, or both of R₃ together are CH₂CH₂.

43. A compound according to claim 1 of formula Iv, wherein R₁ and R₂ are independently selected from H, D, Cl, and F.

44. A compound according to claim 1 of formula Iw 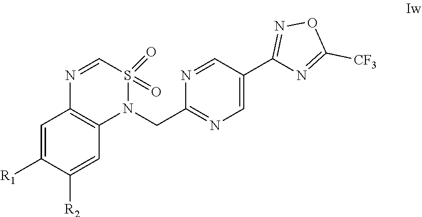
wherein R₁ and R₂ are defined as in claim 1.

45. A compound according to claim 1 of formula Iw, wherein R₁ and R₂ are independently selected from H, D, Cl, and F.

46. A compound according to claim 1 of formula Ix 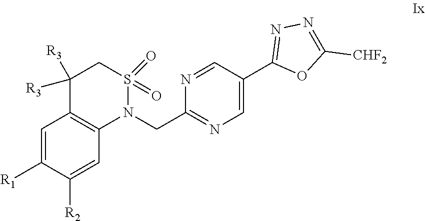
wherein R₁ and R₂ are defined as in claim 1, and both of R₃ are methyl, or one of R₃ is methyl and the other is H, or both of R₃ together are CH₂CH₂ or CH₂CH₂CH₂.

47. A compound according to claim 1 of formula Ix, wherein R₁ and R₂ are independently selected from H, D, Cl, and F.

48. A compound according to claim 1 of formula Iy 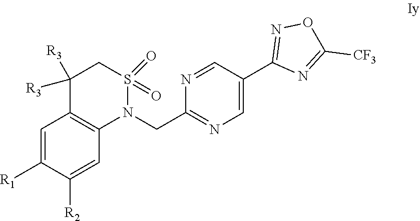
wherein R₁ and R₂ are defined as in claim 1, and both of R₃ are methyl, or one of R₃ is methyl and the other is H, or both of R₃ together are CH₂CH₂ or CH₂CH₂CH₂.

49. A compound according to claim 1 of formula Iy, wherein R₁ and R₂ are independently selected from H, D, Cl, and F.

50. A compound according to claim 1 of formula Iz 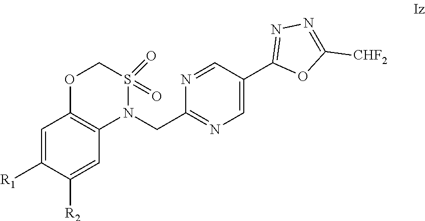
wherein R₁ and R₂ are defined as in claim 1.

51. A compound according to claim 1 of formula Iz, wherein R₁ and R₂ are independently selected from H, D, Cl, and F.

52. A compound according to claim 1 of formula Iaa 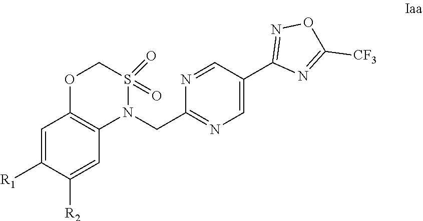
wherein R₁ and R₂ are defined as in claim 1.

53. A compound according to claim 1 of formula Iaa, wherein R₁ and R₂ are independently selected from H, D, Cl, and F.

54. A compound according to claim 1 of formula Ibb 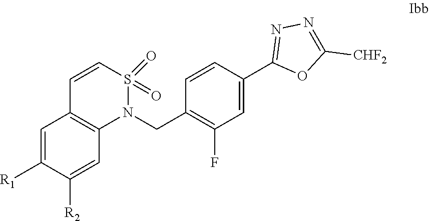
wherein R₁ and R₂ are defined as in claim 1.

55. A compound according to claim 1 of formula Ibb, wherein R₁ and R₂ are independently selected from H, D, Cl, and F.

56. A compound according to claim 1 of formula Icc 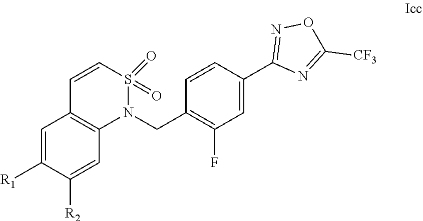
wherein R₁ and R₂ are defined as in claim 1.

57. A compound according to claim 1 of formula Icc, wherein R₁ and R₂ are independently selected from H, D, Cl, and F.

58. A compound according to claim 1 of formula Idd 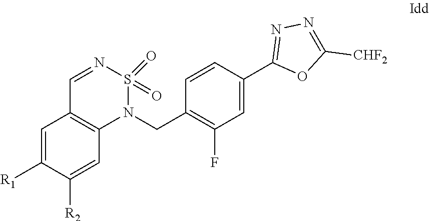
wherein R₁ and R₂ are defined as in claim 1.

59. A compound according to claim 1 of formula Idd, wherein R₁ and R₂ are independently selected from H, D, Cl, and F.

60. A compound according to claim 1 of formula lee 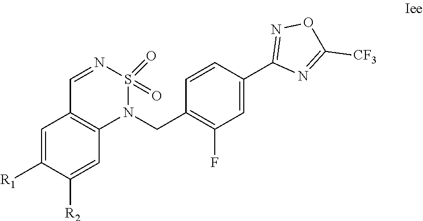
wherein R₁ and R₂ are defined as in claim 1.

61. A compound according to claim 1 of formula lee, wherein R₁ and R₂ are independently selected from H, D, Cl, and F.

62. A compound according to claim 1 of formula Iff 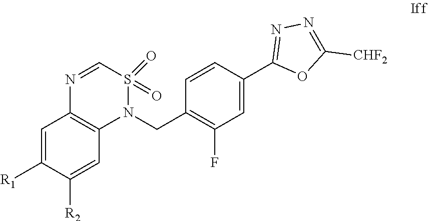
wherein R₁ and R₂ are defined as in claim 1, and R₃ is methyl, or both of R₃ together are CH₂CH₂.

63. A compound according to claim 1 of formula Iff, wherein R₁ and R₂ are independently selected from H, D, Cl, and F.

64. A compound according to claim 1 of formula Igg 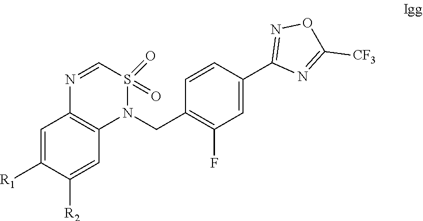
wherein R₁ and R₂ are defined as in claim 1.

65. A compound according to claim 1 of formula Igg, wherein R₁ and R₂ are independently selected from H, D, Cl, and F.

66. A compound according to claim 1 of formula Ihh 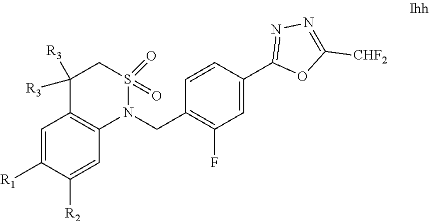
wherein R₁ and R₂ are defined as in claim 1, and both of R₃ are methyl, or one of R₃ is methyl and the other is H, or both of R₃ together are CH₂CH₂ or CH₂CH₂CH₂.

67. A compound according to claim 1 of formula Ihh, wherein R₁ and R₂ are independently selected from H, D, Cl, and F.

68. A compound according to claim 1 of formula Iii 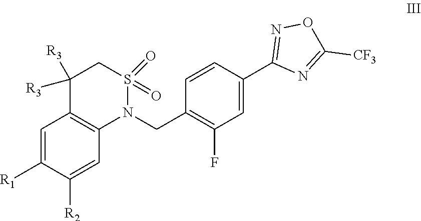
wherein R₁ and R₂ are defined as in claim 1, and both of R₃ are methyl, or one of R₃ is methyl and the other is H, or both of R₃ together are CH₂CH₂ or CH₂CH₂CH₂.

69. A compound according to claim 1 of formula III, wherein R₁ and R₂ are independently selected from H, D, Cl, and F.

70. A compound according to claim 1 of formula Ijj 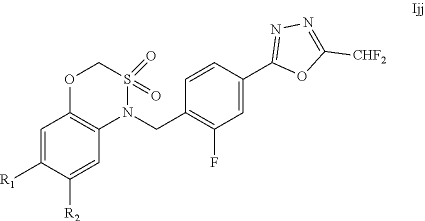
wherein R₁ and R₂ are defined as in claim 1.

71. A compound according to claim 1 of formula Ijj, wherein R₁ and R₂ are independently selected from H, D, Cl, and F.

72. A compound according to claim 1 of formula Ikk 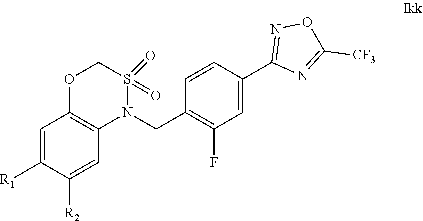
wherein R₁ and R₂ are defined as in claim 1.

73. A compound according to claim 1 of formula Ikk, wherein R₁ and R₂ are independently selected from H, D, Cl, and F.

74. A compound according to claim 1 of formula ILL 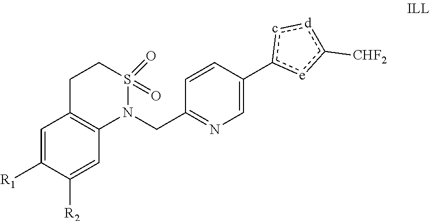
wherein [symbol]
represents a single or double bond; of the five bonds thus marked, two are double bonds and three are single bonds, together arranged in such manner as to give rise to a five-membered ring heteroaryl group;
one of c, d, and e is oxygen, and the remainder are nitrogen; or c is C(R_{f}), one of d and e is oxygen, and the other is nitrogen;
R_{f} is hydrogen, deuterium, fluorine, chlorine, methyl, difluoromethyl, trifluoromethyl, or cyano;
and R₁ and R₂ are defined as in claim 1.

75. A compound according to claim 1 of formula ILL, wherein R₁ and R₂ are independently selected from H, D, Cl, and F, c and d are nitrogen, and e is oxygen.

76. A compound according to claim 1 of formula Imm 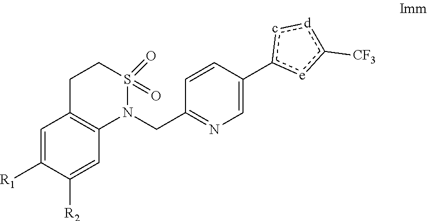
wherein [symbol]
represents a single or double bond; of the five bonds thus marked, two are double bonds and three are single bonds, together arranged in such manner as to give rise to a five-membered ring heteroaryl group;
one of c, d, and e is oxygen, and the remainder are nitrogen; or c is C(R_{f}), one of d and e is oxygen, and the other is nitrogen;
R_{f} is hydrogen, deuterium, fluorine, chlorine, methyl, difluoromethyl, trifluoromethyl, or cyano;
R₁ and R₂ are defined as in claim 1.

77. A compound according to claim 1 of formula Imm, wherein R₁ and R₂ are independently selected from H, D, Cl, and F, c and e are nitrogen, and d is oxygen.

78. A compound according to claim 1 of formula Inn 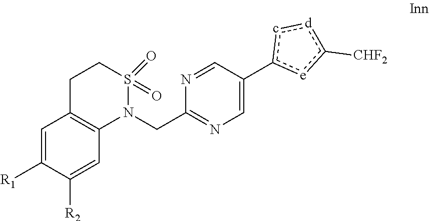
wherein [symbol]
represents a single or double bond; of the five bonds thus marked, two are double bonds and three are single bonds, together arranged in such manner as to give rise to a five-membered ring heteroaryl group;
one of c, d, and e is oxygen, and the remainder are nitrogen; or c is C(R_{f}), one of d and e is oxygen, and the other is nitrogen;
R_{f} is hydrogen, deuterium, fluorine, chlorine, methyl, difluoromethyl, trifluoromethyl, or cyano;
and R₁ and R₂ are defined as in claim 1.

79. A compound according to claim 1 of formula Inn, wherein R₁ and R₂ are independently selected from H, D, Cl, and F, c and d are nitrogen, and e is oxygen.

80. A compound according to claim 1 of formula Ioo 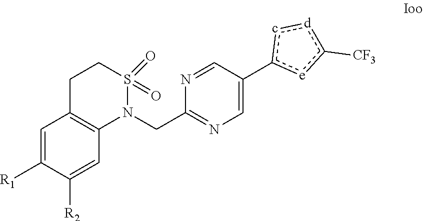
wherein [symbol]
represents a single or double bond; of the five bonds thus marked, two are double bonds and three are single bonds, together arranged in such manner as to give rise to a five-membered ring heteroaryl group;
one of c, d, and e is oxygen, and the remainder are nitrogen; or c is C(R_{f}), one of d and e is oxygen, and the other is nitrogen;
R_{f} is hydrogen, deuterium, fluorine, chlorine, methyl, difluoromethyl, trifluoromethyl, or cyano;
and R₁ and R₂ are defined as in claim 1.

81. A compound according to claim 1 of formula Ioo, wherein R₁ and R₂ are independently selected from H, D, Cl, and F, c and e are nitrogen, and d is oxygen.

82. A compound according to claim 1 of formula Ipp 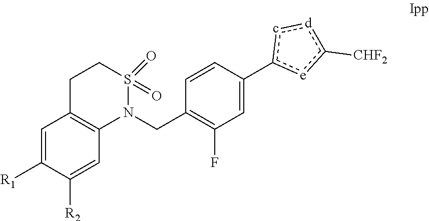
wherein [symbol]
represents a single or double bond; of the five bonds thus marked, two are double bonds and three are single bonds, together arranged in such manner as to give rise to a five-membered ring heteroaryl group;
one of c, d, and e is oxygen, and the remainder are nitrogen; or c is C(R_{f}), one of d and e is oxygen, and the other is nitrogen;
R_{f} is hydrogen, deuterium, fluorine, chlorine, methyl, difluoromethyl, trifluoromethyl, or cyano;
and R₁ and R₂ are defined as in claim 1.

83. A compound according to claim 1 of formula Ipp, wherein R₁ and R₂ are independently selected from H, D, Cl, and F, c and d are nitrogen, and e is oxygen.

84. A compound according to claim 1 of formula Iqq 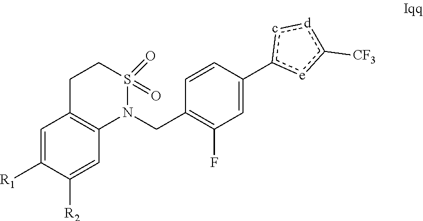
wherein
one of c, d, and e is oxygen, and the remainder are nitrogen; or c is C(R_{f}), one of d and e is oxygen, and the other is nitrogen;
R_{f} is hydrogen, deuterium, fluorine, chlorine, methyl, difluoromethyl, trifluoromethyl, or cyano;
and R₁ and R₂ are defined as in claim 1.

85. A compound according to claim 1 of formula Iqq, wherein R₁ and R₂ are independently selected from H, D, Cl, and F, c and e are nitrogen, and d is oxygen.

86. A compound of claim 1, wherein the compound is selected from the group consisting of:

| Ex | |
|---|---|
| am- | |
| ple | |
| No. | Structure |
| 1 | 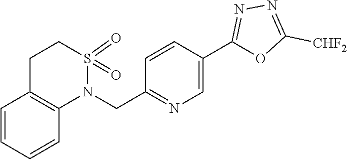 [SMILES: O=S1(=O)CCc2ccccc2N1Cc1ccc(-c2nnc(C(F)F)o2)cn1] |
| 2 | 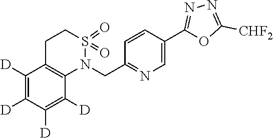 [SMILES: [2H]c1c([2H])c([2H])c2c(c1[2H])CCS(=O)(=O)N2Cc1ccc(-c2nnc(C(F)F)o2)cn1] |
| 3 | 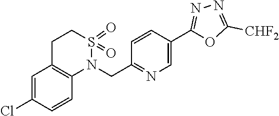 [SMILES: O=S1(=O)CCc2cc(Cl)ccc2N1Cc1ccc(-c2nnc(C(F)F)o2)cn1] |
| 4 | 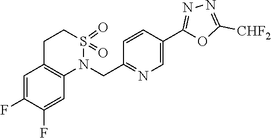 [SMILES: O=S1(=O)CCc2cc(F)c(F)cc2N1Cc1ccc(-c2nnc(C(F)F)o2)cn1] |
| 5 | 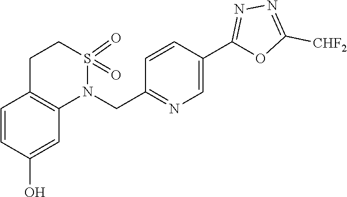 [SMILES: O=S1(=O)CCc2ccc(O)cc2N1Cc1ccc(-c2nnc(C(F)F)o2)cn1] |
| 6 | 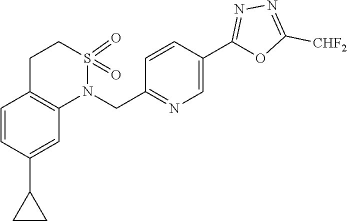 [SMILES: O=S1(=O)CCc2ccc(C3CC3)cc2N1Cc1ccc(-c2nnc(C(F)F)o2)cn1] |
| 7 | 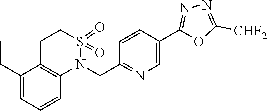 [SMILES: CCc1cccc2c1CCS(=O)(=O)N2Cc1ccc(-c2nnc(C(F)F)o2)cn1] |
| 8 | 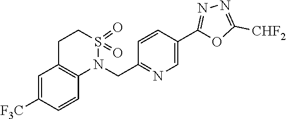 [SMILES: O=S1(=O)CCc2cc(C(F)(F)F)ccc2N1Cc1ccc(-c2nnc(C(F)F)o2)cn1] |
| 9 | 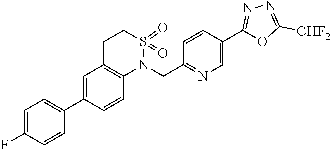 [SMILES: O=S1(=O)CCc2cc(-c3ccc(F)cc3)ccc2N1Cc1ccc(-c2nnc(C(F)F)o2)cn1] |
| 10 | 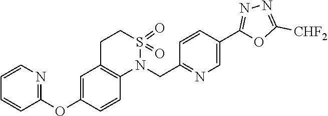 [SMILES: O=S1(=O)CCc2cc(Oc3ccccn3)ccc2N1Cc1ccc(-c2nnc(C(F)F)o2)cn1] |
| 11 | 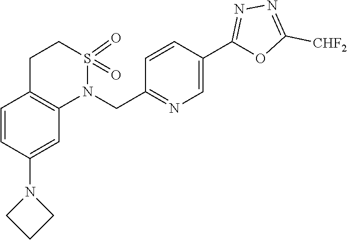 [SMILES: O=S1(=O)CCc2ccc(N3CCC3)cc2N1Cc1ccc(-c2nnc(C(F)F)o2)cn1] |
| 12 | 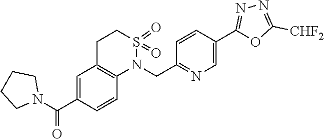 [SMILES: O=C(c1ccc2c(c1)CCS(=O)(=O)N2Cc1ccc(-c2nnc(C(F)F)o2)cn1)N1CCCC1] |
| 13 | 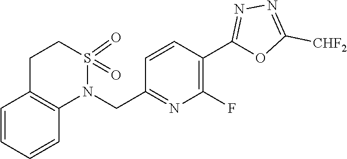 [SMILES: O=S1(=O)CCc2ccccc2N1Cc1ccc(-c2nnc(C(F)F)o2)c(F)n1] |
| 14 | 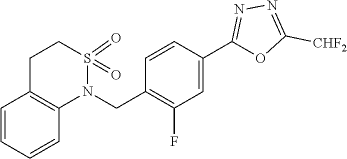 [SMILES: O=S1(=O)CCc2ccccc2N1Cc1ccc(-c2nnc(C(F)F)o2)cc1F] |
| 15 | 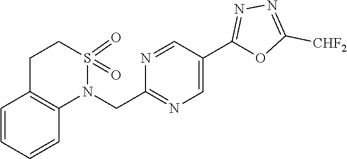 [SMILES: O=S1(=O)CCc2ccccc2N1Cc1ncc(-c2nnc(C(F)F)o2)cn1] |
| 16 | 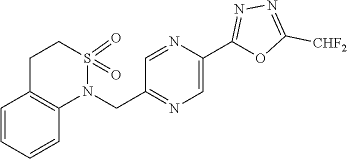 [SMILES: O=S1(=O)CCc2ccccc2N1Cc1cnc(-c2nnc(C(F)F)o2)cn1] |
| 17 | 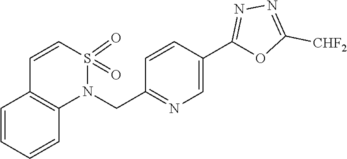 [SMILES: O=S1(=O)C=Cc2ccccc2N1Cc1ccc(-c2nnc(C(F)F)o2)cn1] |
| 18 | 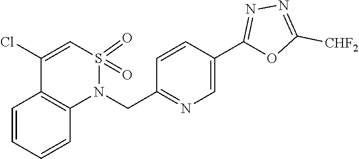 [SMILES: O=S1(=O)C=C(Cl)c2ccccc2N1Cc1ccc(-c2nnc(C(F)F)o2)cn1] |
| 19 | 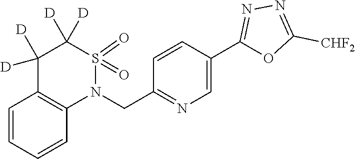 [SMILES: [2H]C1([2H])c2ccccc2N(Cc2ccc(-c3nnc(C(F)F)o3)cn2)S(=O)(=O)C1([2H])[2H]] |
| 20 | 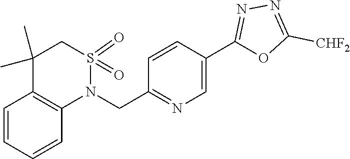 [SMILES: CC1(C)CS(=O)(=O)N(Cc2ccc(-c3nnc(C(F)F)o3)cn2)c2ccccc21] |
| 21 | 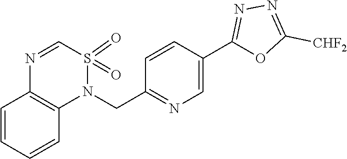 [SMILES: O=S1(=O)C=Nc2ccccc2N1Cc1ccc(-c2nnc(C(F)F)o2)cn1] |
| 22 | 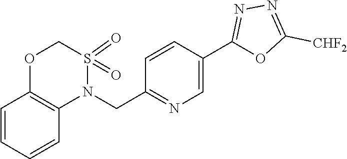 [SMILES: O=S1(=O)COc2ccccc2N1Cc1ccc(-c2nnc(C(F)F)o2)cn1] |
| 23 | 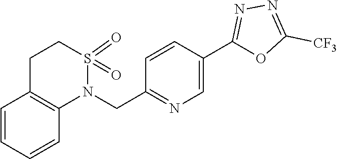 [SMILES: O=S1(=O)CCc2ccccc2N1Cc1ccc(-c2nnc(C(F)(F)F)o2)cn1] |
| 24 | 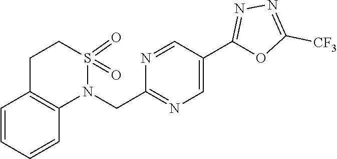 [SMILES: O=S1(=O)CCc2ccccc2N1Cc1ncc(-c2nnc(C(F)(F)F)o2)cn1] |
| 25 | 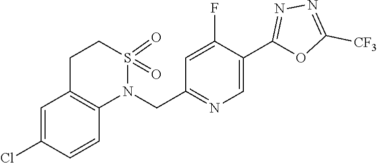 [SMILES: O=S1(=O)CCc2cc(Cl)ccc2N1Cc1cc(F)c(-c2nnc(C(F)(F)F)o2)cn1] |
| 26 | 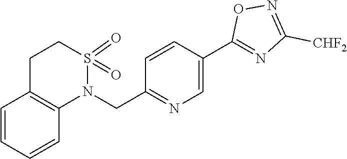 [SMILES: O=S1(=O)CCc2ccccc2N1Cc1ccc(-c2nc(C(F)F)no2)cn1] |
| 27 | 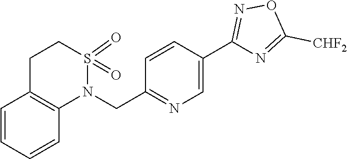 [SMILES: O=S1(=O)CCc2ccccc2N1Cc1ccc(-c2noc(C(F)F)n2)cn1] |
| 28 | 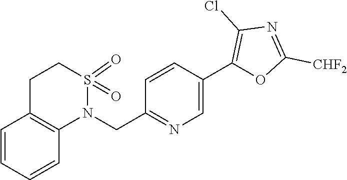 [SMILES: O=S1(=O)CCc2ccccc2N1Cc1ccc(-c2oc(C(F)F)nc2Cl)cn1] |
| 29 | 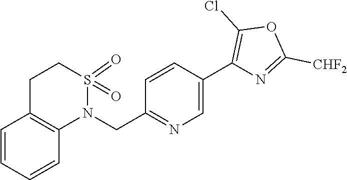 [SMILES: O=S1(=O)CCc2ccccc2N1Cc1ccc(-c2nc(C(F)F)oc2Cl)cn1] |
| 30 | 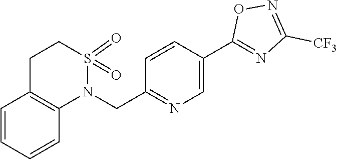 [SMILES: O=S1(=O)CCc2ccccc2N1Cc1ccc(-c2nc(C(F)(F)F)no2)cn1] |
| 31 | 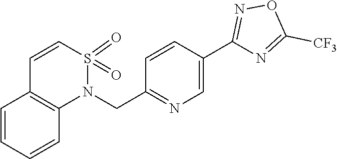 [SMILES: O=S1(=O)C=Cc2ccccc2N1Cc1ccc(-c2noc(C(F)(F)F)n2)cn1] |
| 32 | 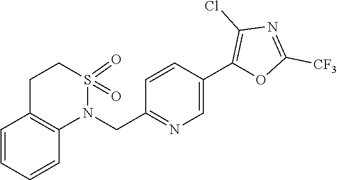 [SMILES: O=S1(=O)CCc2ccccc2N1Cc1ccc(-c2oc(C(F)(F)F)nc2Cl)cn1] |
| 33 | 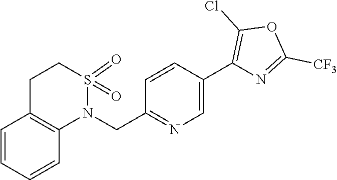 [SMILES: O=S1(=O)CCc2ccccc2N1Cc1ccc(-c2nc(C(F)(F)F)oc2Cl)cn1] |
| 34 | 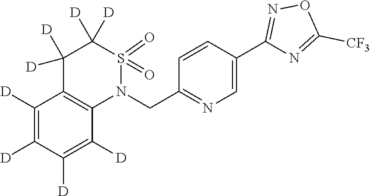 [SMILES: [2H]c1c([2H])c([2H])c2c(c1[2H])N(Cc1ccc(-c3noc(C(F)(F)F)n3)cn1)S(=O)(=O)C([2H])([2H])C2([2H])[2H]] |
| 35 | 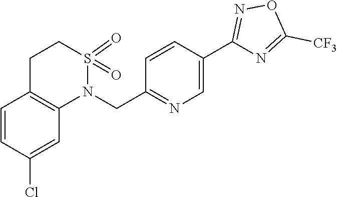 [SMILES: O=S1(=O)CCc2ccc(Cl)cc2N1Cc1ccc(-c2noc(C(F)(F)F)n2)cn1] |
| 36 | 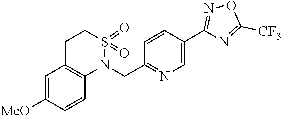 [SMILES: COc1ccc2c(c1)CCS(=O)(=O)N2Cc1ccc(-c2noc(C(F)(F)F)n2)cn1] |
| 37 | 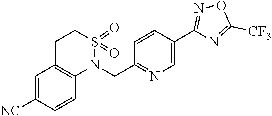 [SMILES: N#Cc1ccc2c(c1)CCS(=O)(=O)N2Cc1ccc(-c2noc(C(F)(F)F)n2)cn1] |
| 38 | 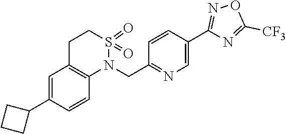 [SMILES: O=S1(=O)CCc2cc(C3CCC3)ccc2N1Cc1ccc(-c2noc(C(F)(F)F)n2)cn1] |
| 39 | 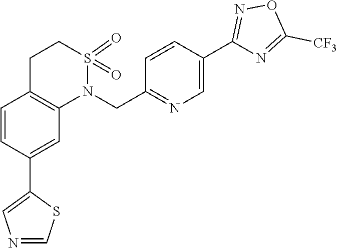 [SMILES: O=S1(=O)CCc2ccc(-c3cncs3)cc2N1Cc1ccc(-c2noc(C(F)(F)F)n2)cn1] |
| 40 | 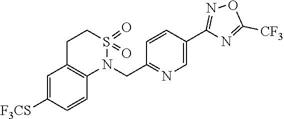 |
| 41 | 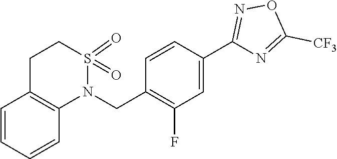 [SMILES: O=S1(=O)CCc2ccccc2N1Cc1ccc(-c2noc(C(F)(F)F)n2)cc1F] |
| 42 | 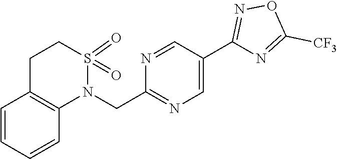 [SMILES: O=S1(=O)CCc2ccccc2N1Cc1ncc(-c2noc(C(F)(F)F)n2)cn1] |
| 43 | 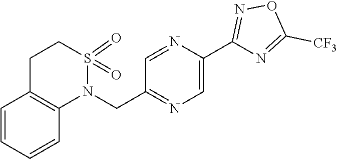 [SMILES: O=S1(=O)CCc2ccccc2N1Cc1cnc(-c2noc(C(F)(F)F)n2)cn1] |
| 44 | 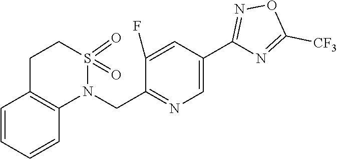 [SMILES: O=S1(=O)CCc2ccccc2N1Cc1ncc(-c2noc(C(F)(F)F)n2)cc1F] |
| 45 | 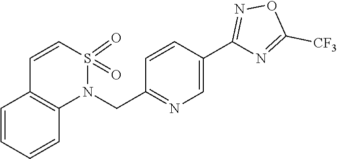 [SMILES: O=S1(=O)C=Cc2ccccc2N1Cc1ccc(-c2noc(C(F)(F)F)n2)cn1] |
| 46 | 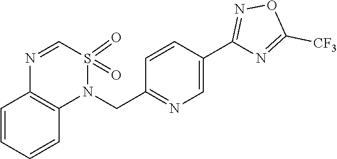 [SMILES: O=S1(=O)C=Nc2ccccc2N1Cc1ccc(-c2noc(C(F)(F)F)n2)cn1] |
| 47 | 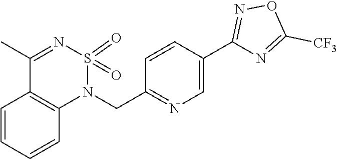 [SMILES: CC1=NS(=O)(=O)N(Cc2ccc(-c3noc(C(F)(F)F)n3)cn2)c2ccccc21] |
| 48 | 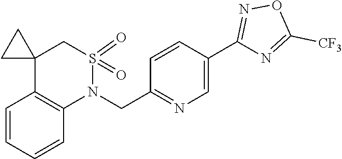 [SMILES: O=S1(=O)CC2(CC2)c2ccccc2N1Cc1ccc(-c2noc(C(F)(F)F)n2)cn1] |
| 49 | 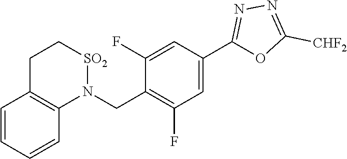 [SMILES: O=S1(=O)CCc2ccccc2N1Cc1c(F)cc(-c2nnc(C(F)F)o2)cc1F] |
| 50 | 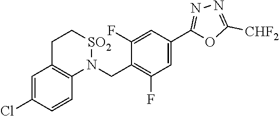 [SMILES: O=S1(=O)CCc2cc(Cl)ccc2N1Cc1c(F)cc(-c2nnc(C(F)F)o2)cc1F] |
| 51 | 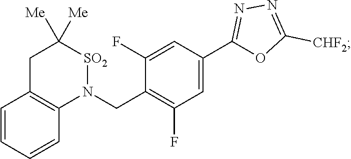 [SMILES: CC1(C)Cc2ccccc2N(Cc2c(F)cc(-c3nnc(C(F)F)o3)cc2F)S1(=O)=O] |

a pharmaceutically acceptable salt thereof.

87. A composition comprising (a) a compound of claims 1-86; (b) a second therapeutic agent useful in the treatment of a disease or condition wherein inhibition of HDAC provides a benefit; and (c) an optional excipient and/or pharmaceutically acceptable carrier.

88. The composition of claim 87 wherein the second therapeutic agent comprises a chemotherapeutic agent useful in the treatment of a cancer.

89. A pharmaceutical composition comprising a compound of claims 1-86 and a pharmaceutically acceptable carrier or vehicle.

90. A method of treating a disease or condition wherein inhibition of HDAC provides a benefit comprising administering a therapeutically effective amount of a compound of claims 1-86 to an individual in need thereof.

91. The method of claim 90 wherein the HDAC is HDAC6.

92. The method of claim 90 further comprising administering a therapeutically effective amount of a second therapeutic agent useful in the treatment of the disease or condition.

93. The method of claim 92 wherein the compound of claims 1-86 and the second therapeutic agent are administered simultaneously.

94. The method of claim 92 wherein the compound of claims 1-86 and the second therapeutic agent are administered separately.

95. The method of claim 90 wherein the disease or condition is a cancer.

96. The method of claim 92 wherein the disease is a cancer and the second therapeutic agent is one or more of a chemotherapeutic agent, radiation, and an immunotherapy.

97. The method of claim 96 wherein the second therapeutic agent comprises radiation, and the radiation optionally is administered in conjunction with radiosensitizers and/or therapeutic agents.

98. The method of claim 90 wherein the disease or condition is a neurological disease, a neurodegenerative disorder, peripheral neuropathy, or a traumatic brain injury.

99. The method of claim 90 wherein the disease or condition is a stroke.

100. The method of claim 90 wherein the disease or condition is an inflammation or an autoimmune disease.

101. The method of claim 100 further comprising administering a therapeutically effective amount of a second therapeutic agent useful in the treatment of the autoimmune disease or the inflammation.

102. A method of increasing sensitivity of a cancer cell to cytotoxic effects of a radiotherapy and/or a chemotherapy comprising contacting the cell with a compound of claim 1-86 in an amount sufficient to increase the sensitivity of the cell to the radiotherapy and/or the chemotherapy.
